(19) United States
(12) Patent Application Publication
(10) Pub. No.: US 2021/0319279 A1
(43) Pub. Date: Oct. 14, 2021

(54) BIOLOGICAL COMPUTING SYSTEMS AND METHODS FOR MULTIVARIATE SURFACE ANALYSIS AND OBJECT DETECTION

(71) Applicant: XENO CELL INNOVATIONS S.R.O., Pilsen (CZ)

(72) Inventors: Daniel Georgiev, Pilsen (CZ); Martin Cienciala, Pilsen (CZ); Hynek Kasl, Pilsen (CZ); Laura Berne, Pilsen (CZ); Tereza Puchrova, Pilsen (CZ)

(21) Appl. No.: 17/268,340

(22) PCT Filed: Sep. 6, 2019
(86) PCT No.: PCT/US19/50068

§ 371 Date: Feb. 12, 2021

Related U.S. Application Data
(60) Provisional application No. 62/728,661, filed on Sep. 7, 2018.

(51) Int. Cl. G06N 3/00

(52) U.S. Cl. CPC G06N 3/002

(57) ABSTRACT

The present invention provides biological computing systems comprising computing units that process input signals to produce an output signal. In particular, the computing units include, for example, cells and proteins that function to convert biological signals into a discernable output that provides information about a biological sample. Further provided are methods of using such biological computing systems, such as for the diagnosis of various diseases and conditions.

### CROSS-REFERENCE TO RELATED APPLICATIONS

This application claims the benefit of priority to U.S. Provisional Patent Application No. 62/728,661, filed Sep. 7, 2018, the disclosure of which is incorporated herein by reference in its entirety.

### FIELD

The present disclosure relates to biological computing systems comprising computing units that process input signals to produce an output signal. In particular, the computing units include, for example, cells and proteins that function to convert biological signals into a discernable output that provides information about a biological sample. Further provided are methods of using such biological computing systems, such as for the diagnosis of various diseases and conditions.

### BACKGROUND OF THE INVENTION

Detection of foreign matter is fundamental to the functioning of any organism. As part of their natural immunity, organisms across the phylogenetic spectrum have evolved detection mechanisms of various complexities. Bacteria detect viral transformation using sequence specific enzymes that simultaneously recognize and suppress the intruder. Single cell eukaryotes detect the presence of bacteria using pattern recognition receptors that initiate various cellular responses. Multicellular organisms discriminate between the self and the non-self. This involves detecting many possible phenotypes based on multivariate features amongst a large number of normal cells, a task for which the simpler immune mechanisms alone are insufficient. Hence, higher organisms have additional hierarchical immune systems that distribute recognition tasks amongst many specialized cells each having their own sensory, signaling and memory functions.

Various elements of immune systems are also used in diagnostic technologies. Currently antigen detection is primarily based on molecular technologies that resemble soluble and membrane bound receptors of the vertebrate adaptive immune system. The many takes on the enzyme-linked immunosorbent assay (ELISA) and flow cytometry illustrate the possibilities and limitations of the current state-of-the-art. Both technologies employ antigen optimized antibodies for detection of specific molecular structures. The technologies differ in target quantification. ELISA is based on target signal amplification and flow cytometry relies on brute force screening of individual cells. Both methods, however, generate output signals that are directly generated by the target-antibody complex. This holds true even in variations where soluble targets are assembled into complexes in multiple stacking steps where cooperativity stands to improve detection specificity. It should also be mentioned hybrid solutions exist wherein sample enrichment (e.g., through immunochemical capture or size exclusion) is combined with brute-force screening to achieve various tradeoffs between throughput and specificity.

In general, current diagnostic technologies are comparable to the initial excerpt of natural immune mechanisms. The hierarchical computing systems that process target-receptor interactions and significantly increase the accuracy and reliability of detection have yet to find their technological parallels. In the absence of these computing systems, detection accuracy of immunological mechanisms is limited by the receptor-target binding kinetics. For example, one can consider detecting a group of diseased cells in a sample the size of the human body by either flow cytometry, which would take half a year, or by ELISA, which would require femtomolar antibody sensitivity and absolute specificity. The adaptive immune system solves the same problem in a week's time using only low-affinity antibodies.

Detection efficiency is primarily achieved by immune computing systems that are far more than happenstance collusions of individual cell types. Indeed, phylogenetic studies reporting convergent evolution patterns and functional reconstructions from allogenic cell types suggest that computing architectures have a mechanistic value. Although we are far from fully understanding the design, accepted theories, e.g., idiotypic network theory and clonal selection, as well as omics data provide some information regarding various levels of immune cell computation. At the intracellular levels, receptor modifications, kinase cascades, genetic regulators, etc., act to increase specificity, gate signals, and store cellular states. At the extracellular levels, cytokines, antimicrobial peptides, interferons, etc., act to coordinate, stabilize, and reset cell states. At the cell-to-cell interfaces, phenotypic surface markers (e.g., cluster of differentiation molecules, anti-antibody variable regions, stress proteins, antigen recognizing fragments) generate cell-to-cell interactions that lead to localized signaling and subsequent cell state changes (e.g., activation, suppression, anergy, apoptosis).

Direct utilization of immune computing systems in diagnostics may be unsuitable and remains out of reach. Nonetheless, simple biological or biologically compatible systems are already being coopted for similar computing functions using bottom up approaches. Working examples from synthetic biology include a wide array of intracellular logic and regulatory devices (e.g., logic gates, switches, memory storage, oscillators, insulators, counters) engineered through rewiring of genetic networks and repurposing of molecular machinery. Coordination of devices operating in different cells is also made possible using known intercellular signaling systems (e.g., quorum sensing, pheromone response, volatile production). Some cell free solutions include DNA computers or functionalized nanoparticles wherein molecular binding and binding site obstruction are orchestrated to detect and logically process soluble analytes (e.g., DNA oligos, antigens, enzymes). In all cases, the technologies are demonstrated either in homogeneous cultures or, less commonly, in adherent or otherwise immobilized configurations. Hence, applications are limited to cases wherein the relative positioning of the computational devices is considered, for all design purposes, statistically constant throughout the detection process. Some application examples fitting these specifications include various biosensors for detection of soluble compounds (e.g., contaminants, pollutants, metabolites) that can function in either standalone solutions or within larger devices (e.g., therapeutic delivery systems, bioremediators, cell factories).

### SUMMARY

Several aspects described herein relate to biological computing systems, methods of their preparation, and methods of using such systems.

In one aspect, provided herein is a system for biological computing, comprising: one or more computing units (CUs), wherein the one or more CUs are configured to interact with one or more sample objects derived from an input sample such that an output signal indicative of a characteristic of the input sample is generated.

In some embodiments, the system further comprises the one or more sample objects.

In some embodiments, the system is configured such that one or more computational clusters can be formed, wherein each computational cluster comprises, independently, one or more of the CUs.

In some embodiments, one or more of the computational clusters further comprise, independently, one or more of the sample objects.

In some embodiments, one or more of the computational clusters are formed by diffusion of their components in a medium comprising the system such that the components associate with one another.

In some embodiments, one or more of the computational clusters are formed by application of a force to a medium comprising the system such that their components are placed in proximity to one another, thereby facilitating their association.

In some embodiments, the output signal comprises a level of an output signal object (SO).

In some embodiments, each of the one or more CUs is, independently, i) associated with one or more surface-bound entities (SBEs); ii) capable of recognizing an SO; iii) capable of producing an SO; iv) capable of degrading an SO; v) capable of producing a change in a material property of a medium comprising the system; and/or vi) capable of producing another CU.

In some embodiments, an SBE associated with a CU in the system is capable of binding to a cognate binding partner.

In some embodiments, the cognate binding partner is an SO or another SBE.

In some embodiments, an SBE associated with a CU in the system comprises an SO.

In some embodiments, association of a CU in the system with an SBE comprises covalent attachment of the SBE to a surface of the CU or non-covalent attachment of the SBE to a surface of the CU.

In some embodiments, the SBE is covalently attached to the surface of the CU by a linker.

In some embodiments, a CU in the system is capable of producing an SO such that the SO is released into a medium comprising the system.

In some embodiments, a CU in the system is capable of producing an SO such that the SO is associated with the CU.

In some embodiments, a CU in the system is capable of producing an SO, and the SO is an internal SO recognized by at least one of the one or more CUs and/or at least one of the one or more sample objects.

In some embodiments, a CU in the system is capable of producing an SO, the SO is an output SO, and the output signal comprises a level of the output SO.

In some embodiments, the capability of a CU in the system to produce an SO is enhanced by binding of an SBE associated with the CU to its cognate binding partner.

In some embodiments, the cognate binding partner is an SBE associated with one of the one or more CUs, an SBE associated with one of the one or more sample objects, or another SO.

In some embodiments, the capability of a CU in the system to produce an SO is attenuated by binding of an SBE associated with the CU to its cognate binding partner.

In some embodiments, the cognate binding partner is an SBE associated with one of the one or more CUs, an SBE associated with one of the one or more sample objects, or another SO.

In some embodiments, a CU in the system is capable of degrading an SO, and the SO is an internal SO recognized by at least one of the one or more CUs and/or at least one of the one or more sample objects.

In some embodiments, a CU in the system is capable of degrading an SO, the SO is an output SO, and the output signal comprises a level of the output SO.

In some embodiments, the capability of a CU in the system to degrade an SO is enhanced by binding of an SBE associated with the CU to its cognate binding partner.

In some embodiments, the cognate binding partner is an SBE associated with one of the one or more CUs, an SBE associated with one of the one or more sample objects, or another SO.

In some embodiments, the capability of a CU in the system to degrade an SO is attenuated by binding of an SBE associated with the CU to its cognate binding partner.

In some embodiments, the cognate binding partner is an SBE associated with one of the one or more CUs, an SBE associated with one of the one or more sample objects, or another SO.

In some embodiments, the material property of the medium is an optical property, an electrical property, or a thermal property.

In some embodiments, a CU in the system that is capable of producing another CU is capable of producing a CU of the same type as the CU or a CU of a different type as the CU.

In some embodiments, a first CU of the one or more CUs is associated with a first SBE, a second CU of the one or more CUs is associated with a second SBE, and the first and second SBEs are capable of forming a complex comprising the first and second SBEs.

In some embodiments, each of the one or more CUs comprises, independently, a cell or a molecule.

In some embodiments, a CU in the system comprises a cell, and an SBE is covalently attached to an anchor present in the cell.

In some embodiments, the SBE is covalently attached to the anchor by a linker.

In some embodiments, the linker comprises a repeat motif.

In some embodiments, the linker facilitates accessibility of the SBE and/or increases an effective contact area between sample objects and the CU.

In some embodiments, a CU in the system comprises a molecule selected from a polypeptide, a polypeptide derivative, a nucleic acid, and a solid support.

In some embodiments, the polypeptide is a bispecific antibody (BsAb).

In some embodiments, the polypeptide is an enzyme.

In some embodiments, the enzyme is capable of converting an agent in a medium comprising the system into an SO.

In some embodiments, the SO is recognized by another of the one or more CUs and/or the output signal comprises the SO.

In some embodiments, the solid support is a functionalized bead.

In some embodiments, each of the one or more sample objects is, independently, i) associated with one or more SBEs; ii) capable of recognizing an SO; iii) capable of producing an SO; iv) capable of degrading an SO; v) capable of producing a change in a material property of a medium comprising the system; and/or vi) capable of producing another sample object.

In some embodiments, a sample object in the system is capable of producing an SO, and the SO is an internal SO recognized by at least one of the one or more CUs and/or at least one of the one or more sample objects.

In some embodiments, a sample object in the system is capable of producing an SO, the SO is an output SO, and the output signal comprises a level of the output SO.

In some embodiments, a sample object in the system is capable of recognizing an SO produced by a CU in the system.

In some embodiments, recognition of the SO by the sample object is capable of inducing the sample object to produce another SO.

In some embodiments, the other SO is an internal SO or an output SO.

In some embodiments, each of the one or more sample objects is, independently, a cell or a molecule.

In some embodiments, a sample object in the system is a cell, and an SBE associated with the cell comprises an antigen on the surface of the cell.

In some embodiments, an SBE associated with a sample object in the system is recognized by a first SBE associated with a first CU of the one or more CUs.

In some embodiments, the SBE associated with the sample object is directly recognized by the first SBE.

In some embodiments, the SBE associated with the sample object is indirectly recognized by the first SBE through a second CU.

In some embodiments, the SBE associated with the sample object is recognized by a second SBE associated with the second CU, and a third SBE associated with the second CU is recognized by the first SBE.

In some embodiments, the second CU is a BsAb comprising a first binding moiety specific for the SBE associated with the sample object and a second binding moiety specific for the first SBE.

In some embodiments, the system comprises a logical OR module comprising a first CU, wherein the first CU comprises a first CU SBE and a second CU SBE, the first CU SBE is capable of binding to a first cognate binding partner and the second CU SBE is capable of binding to a second cognate binding partner, and 1) the first CU is capable of producing a first SO, binding of the first CU SBE to the first cognate binding partner enhances the capability of the first CU to produce the first SO and binding of the second CU SBE to the second cognate binding partner enhances the capability of the first CU to produce the first SO; or 2) the first CU is capable of degrading a first SO, binding of the first CU SBE to the first cognate binding partner enhances the capability of the first CU to degrade the first SO and binding of the second CU SBE to the second cognate binding partner enhances the capability of the first CU to degrade the first SO.

In some embodiments, the first cognate binding partner is a first sample object SBE associated with at least one of the one or more sample objects and the second cognate binding partner is a second sample object SBE associated with at least one of the one or more sample objects.

In some embodiments, the system comprises a logical AND module comprising a first CU and a second CU, wherein the first CU comprises a first CU SBE and the second CU comprises a second CU SBE, the first CU SBE is capable of binding to a first cognate binding partner and the second CU SBE is capable of binding to a second cognate binding partner, wherein the first CU is capable of producing a first SO, the second CU is capable of producing or degrading a second SO, binding of the first CU SBE to the first cognate binding partner enhances the capability of the first CU to produce the first SO, and recognition of the first SO by the second CU enhances the capability of the second CU to produce or degrade the second SO, and wherein clustering of the first and second cognate binding partners in a computational cluster enhances the effect of the first SO such that the first SO produced by the first CU bound to the first cognate binding partner is capable of inducing the second CU bound to the second cognate binding partner to produce or degrade the second SO.

In some embodiments, the first cognate binding partner is a first sample object SBE associated with a sample object in the system and the second cognate binding partner is a second sample object SBE associated with the sample object.

In some embodiments, the system comprises a logical negated implication module comprising a first CU, a second CU, and a third CU, wherein the first CU comprises a first CU SBE, the second CU comprises a second CU SBE, the third CU comprises the first CU SBE, the first CU SBE is capable of binding to a first cognate binding partner, and the second CU SBE is capable of binding to a second cognate binding partner, wherein the first CU is capable of producing a first SO, the second CU is capable of degrading the first SO, the third CU is capable of producing or degrading a second SO, binding of the first CU SBE of the first CU to the first cognate binding partner enhances the capability of the first CU to produce the first SO, binding of the second CU SBE of the second CU to the second cognate binding partner enhances the capability of the second CU to degrade the first SO, and recognition of the first SO by the third CU enhances the capability of the third CU to produce or degrade the second SO, and wherein clustering of the first cognate binding partners enhances the effect of the first SO such that the first SO produced by the first CU bound to the first cognate binding partner is capable of inducing the third CU bound to the first cognate binding partner to produce or degrade the second SO; and clustering of the first and second cognate binding partners in a computational cluster enhances the effect of the second CU such that the second CU bound to the second cognate binding partner is capable of attenuating induction of third CU by degrading the first SO produced by the first CU bound to the first binding partner.

In some embodiments, the first cognate binding partner is a first sample object SBE associated with a sample object in the system and the second cognate binding partner is a second sample object SBE associated with the sample object.

In some embodiments, the system comprises one or more modules selected from logical OR modules, logical AND modules, and logical negated implication modules.

In some embodiments, a CU in the system is configured to respond to an SO in the system by enhancing the level of the SO.

In some embodiments, the SO is an internal SO or an output SO.

In some embodiments, the CU is capable of producing the SO, and interaction of the SO with the CU enhances the capability of the CU to produce the SO.

In some embodiments, the CU is configured to respond to the SO by producing another SO, and interaction of the other SO with another CU that is capable of producing the SO enhances the capability of the other CU to produce the SO.

In some embodiments, a CU in a computational cluster in the system is configured to respond to an SO present in a boundary layer around the computational cluster by producing another SO that is not restricted to the boundary layer.

In some embodiments, a CU that is not localized to a computational cluster in a medium comprising the system is configured to degrade an SO produced by another CU present in the computational cluster.

In some embodiments, the system further comprises an agent that increases the viscosity of a medium comprising the system.

In some embodiments, the agent is a polymer.

In some embodiments, the polymer is a polysaccharide.

In some embodiments, the agent is cross-linked to form a matrix configured to immobilize one or more of the system components.

In some embodiments, the characteristic of the input sample comprises an indication of whether a target object is present or absent in the input sample.

In some embodiments, the characteristic of the input sample further comprises a quantification of the level of the target object in the input sample

In some embodiments, the input sample is a biological sample derived from an individual.

In some embodiments, the target object is a target cell.

In some embodiments, the target cell is a disease cell, a fetal cell, or a stem cell.

In some embodiments, the target cell is a state-specific target cell.

In some embodiments, the target object is a target pathogen.

In some embodiments, the target object is indicative of a disease or condition in the individual, and the characteristic of the biological sample further comprises a diagnosis of the disease or condition in the individual.

In some embodiments, the input sample is an environmental sample.

In some embodiments, the input sample is a synthetic sample.

In another aspect, provided herein is a composition comprising a system according to any of the embodiments described above suspended in a medium.

In some embodiments, the medium is a liquid or a gas.

In some embodiments, the medium is an aqueous medium.

In another aspect, provided herein is a method of detecting the presence or absence of a target object in an input sample comprising: a) incubating a composition according to any of the embodiments described above for a sufficient amount of time for the output signal to be generated; and b) detecting the output signal, thereby detecting the presence or absence of the target object in the input sample.

In another aspect, provided herein is a method of quantifying the level of a target object in an input sample comprising: a) incubating a composition according to any of the embodiments described above for a sufficient amount of time for the output signal to be generated; and b) detecting the output signal, thereby quantifying the level of the target object in the input sample.

In another aspect, provided herein is a method of diagnosing a disease or condition in an individual, comprising: a) incubating a composition comprising a system according to any of the embodiments described above for a sufficient amount of time for the output signal to be generated; and b) detecting the output signal, thereby diagnosing the disease or condition in the individual.

### BRIEF DESCRIPTION OF THE DRAWINGS

FIGS. 1A-1F show illustrations of individual features of computing units (CUs). FIG. 1A shows a CU with surface-bound entities (SBEs) that are covalently or noncovalently bound to the CU surface. FIG. 1B shows a CU that produces a signal object (SO). FIG. 1C shows a CU that recognizes a specific SO, such as by interaction with an SBE associated with the CU. FIG. 1D shows a CU with an SO that is either covalently or noncovalently bound to the CU surface. FIG. 1E shows a CU that degrades an SO. FIG. 1F shows a CU that produces another CU. A CU as depicted here (and in any of the subsequent FIGS) can include, for example, a cell, a polypeptide (such as a bispecific antibody), a functionalized bead, and the like.

FIGS. 2A and 2B show illustrations of CUs with SBEs interacting with their cognate binding partners. FIG. 2A shows a CU with a first SBE (2) interacting with a first cognate binding partner (1). FIG. 2B shows a CU with a first SBE (2) and a second SBE (3), where (2) interacts with a first cognate binding partner (1), and (3) interacts with a second cognate binding partner (4). (1) is shown associated with a cell membrane, and (4) is shown associated with another CU, though these can be associated with any component present in the system.

FIG. 3 shows a CU (such as a cellular CU) with an SBE that is a construct including an anchoring domain (4) linked to a binding moiety (2) by a linker (3), where (2) interacts with a cognate binding partner (1). For example, the SBE can be a fusion protein. (1) is shown associated with a cell membrane, but can be associated with any component present in the system.

FIG. 4 shows a CU (1) that produces an SO (3) and a CU (2) that recognizes an SO (4). (1) can be, for example, a cell that secretes (3) or an enzyme that cleaves an intermediate agent to produce (3). (3) can be released into the medium of the system or remain associated (covalently or non-covalently) with (1). (2) can be, for example, a cell with a cell surface receptor that binds to (4). (4) can be, for example, an internal SO produced by a CU of the system, or an SO present in a biological sample included in the system.

FIG. 5 shows a CU (1) that changes a material property (3) of the system and a CU (2) that responds to a change in a material property (4) of the system. Material properties (3) and (4) can include, for example, optical, electrical, and thermal properties of the system.

FIG. 6 shows a CU (1) that recognizes an SO derived from a sample object (3) present in a biological sample included in the system and a CU (2) that produces an SO that influences the state of a sample object (4) present in a biological sample included in the system. The SO produced by (2) can be released into the medium of the system or remain associated (covalently or non-covalently) with (2).

FIG. 7 shows a CU (1) that recognizes an SO produced by a CU (3) and a CU (2) that recognizes an SO (4) and produces an SO (5). SO (4) may be an external or internal SO. All SOs that are produced may be either released into the medium or tethered/bound to the outer surface of the CU from which it is produced.

FIG. 8 shows a CU (2) that recognizes an SBE (1) associated with a sample object and that is capable of modulating SO (3) levels. In direct transposition (4), recognition of SBE (1) by CU (2) results in production of SO (3), for example by CU (2). In negated transposition (5), CU (2) can be configured, for example, to i) produce SO (3) only in the absence of recognition of SBE (1); or ii) degrade SO (3) when recognizing SBE (1).

FIG. 9 shows an exemplary logical OR operator module having CU (5), which is associated with SBE (3) capable of recognizing SBE (1), and SBE (4) capable of recognizing SBE (2). In operation (7), CU (5) produces SO (6) when SBE (1) or SBE (2) is present. In operation (8), CU (5) degrades SO (6) when SBE (1) or SBE (2) is present.

FIG. 10 shows an exemplary logical AND operator module having CU (5), which is capable of producing SO (7) and is associated with SBE (3) capable of recognizing SBE (1), and CU (6), which is capable of recognizing SO (7) and modulating SO (8) levels, and is associated with SBE (4) capable of recognizing SBE (2). In operation (9), CU (6) produces SO (8) only when both SBE (1) and SBE (2) are present. In operation (10), CU (6) degrades SO (8) only when both SBE (1) and SBE (2) are present.

FIG. 11 shows an exemplary logical NEGATED IMPLICATION operator module having CU (5), which is capable of producing SO (8) and is associated with SBE (3) capable of recognizing SBE (1), CU (6), which is capable of degrading SO (8) and is associated with SBE (4) capable of recognizing SBE (2), and CU (7), which is capable of recognizing SO (8) and modulating SO (9) levels, and is associated with SBE (3) capable of recognizing SBE (1). In operation (10), CU (7) produces SO (9) only when SBE (1) is present and SBE (2) is not present. In operation (11), CU (7) degrades SO (9) only when SBE (1) is present and SBE (2) is not present.

FIG. 12 shows an example of how transposition of SBEs into SOs may be used to construct an arbitrary logical operator expressed in the sum-of-products (SOP) standard form. The logical operation Σⱼ Π_{i∈N}_{ⱼ}bᵢΠ_{i∈M}_{ⱼ}b̅_̅{̅ι̅}̅, where bᵢ corresponds to type bᵢ SBEs, may be realized by the shown CU types, where type (i,j,P) CUs recognize type bᵢ; i∈Nⱼ, SBEs and type (j,N) CUs recognize any SBEs of the type bᵢ; i∈Mⱼ. The signaling network illustrated in the figure is realized by signals so that each regular arrow corresponds to a unique signal type and each blunt arrow corresponds to degradation of the indicated signal type. Open ended arrows terminating the signal cascades correspond to the same signal type that also is the result of the logical operation.

FIG. 13 shows an example of how transposition of SBEs into signal objects may be used to construct an arbitrary logical operator expressed in the product-of-sums (POS) standard form. The logical operation Πⱼ (Σ_{i∈N}_{ⱼ}bᵢ+Σ_{i∈M}_{ⱼ}b̅_̅{̅ι̅}̅), where bᵢ corresponds to type bᵢ SBEs, is re-written as

$$( {Π}_{i ∈ A} {s}_{i , A} ) \overline{( {\sum }_{ι = 1}^{K} \overline{{s}_{ι , P}} {\prod }_{j = 1}^{{N}_{ι}} {s}_{ι , j , N} )} ,$$

where, s_{i,A}, s_{i,P}, and s_{i,j,N} represent the corresponding logical sums of SBE types. The logical operation in the re-written form may be realized by the shown CU types, where type (i,A) CUs recognize all SBEs whose types are included in the logical sum s_{i,A}. Similarly, recognition of SBEs is defined for type (i,j,N) and type (i,P) CUs. The signaling network illustrated in the figure is realized by signals so that each regular arrow corresponds to a unique signal type and each blunt arrow corresponds to degradation of the indicated signal type. The open ended arrow terminating the (1,A), (2,A), . . . , (NA,A) cascade is the result of the logical operation.

FIG. 14 shows a schematic of the pheroimmuno computing architecture that organizes sequential evaluation of pheroimmuno propositions and the corresponding interactions between objects. The inputs (6) and outputs (7) are SBE profiles of individual objects or object clusters. Recognition of a particular SBE type by other SBE types is chiefly promoted in the clustering reaction (1). The computation operations are chiefly executed in the evaluation reaction (2). The evaluation reaction includes a computation block (3), wherein signal types are produced and recognized according to networks that were constructed to implement specific logical operations. The evaluation reaction also includes a control block (4), wherein the integrity and localization or amplification of pheroimmuno propositions is managed by additional control elements. The clustering and evaluation reactions may be performed in a single batch, cycled (5), or combined into a single hybrid reaction. In addition to the outputs (7), the system may also return readouts (8), which are signals measurable by external instruments.

FIGS. 15A and 15B show exemplary control elements for managing the integrity of pheroimmuno propositions. FIG. 15A shows a CU (2), which is capable of recognizing SBE (1) and SO (3), and is capable of producing SO (3). Recognition of SO (3) by CU (2) enhances the capability of CU (2) to produce SO (3). FIG. 15B shows a CU (2), which is capable of recognizing SBE (1) and SO (5), and is capable of producing SO (3), and a CU (4), which is capable of recognizing SBE (1) and SO (3), and is capable of producing SO (5). Recognition by CU (4) of SO (3) enhances the capability of CU (4) to produce SO (5), and recognition by CU (2) of SO (5) enhances the capability of CU (2) to produce SO (3).

FIG. 16 shows exemplary control elements for amplifying pheroimmuno propositions independently of surface-bound entity recognition. CU (1) is present locally around a sample object in a medium, and is capable of converting a global agent (2) in the medium to waste product (3) and SO (4). SO (4) can be recognized by other CUs in the medium, such as CU (5) shown here.

FIG. 17 shows exemplary control elements for insulating computations in one cluster from pheroimmuno propositions of another cluster. CU (5) and CU (6) are localized to a cluster in a medium, and CU (5) is capable of producing SO (7), which can be recognized by CU (6). CU (8), which is not localized to the cluster, is capable of degrading SO (7).

FIG. 18 shows separation of computational clusters by immobilization within a matrix that entraps CUs and/or sample objects, e.g., by size exclusion (1) or by direct association with the matrix elements (2).

FIG. 19 shows an exemplary SBE display cassette. The cassette comprises a yeast shuttle vector made up of a Bacterial plasmid origin (ColE1), AmpR gene for bacterial selection, URA3 gene for yeast selection, recombination domains Homologous Right and Homologous Left for gene integration into the URA3 locus. The cassette also includes a fragment of the final ORF. This fragment begins with the hybrid signaling sequence comprising the Ost1 Pre sequence (initial 22 AA residues of YJL002C ORF, codon optimized for expression in yeast S. cerevisiae) followed by the MF(alpha) Pro sequence (AA residues 19-89 of YPL187W ORF, codon optimized for expression in yeast S. cerevisiae). This fragment is interrupted by a multiple cloning site that surrounds a GFP reporter gene. At the C terminus is a truncate FLO11 (YIR019C) sequence comprising the 1135 residues of the wild type C terminus.

FIG. 20 shows an exemplary expression cassette. The cassette comprises a yeast shuttle vector made up of a Bacterial plasmid origin (ColE1), AmpR gene for bacterial selection, a marker for auxotrophic selection in yeast (e.g., a gene comprising either the URA3, LEU2, HIS3, LYS2, or MET15 ORF), recombination domains Homologous Right and Homologous Left for gene integration into either the ura3, leu2, HO, lys2, or met15 loci. The cassette comprises multiple cloning site that surrounds a GFP reporter gene and is located upstream of the TDH1 Terminator sequence.

FIG. 21 shows an exemplary multigene cassette. The cassette comprises a yeast shuttle vector made up of a Bacterial plasmid origin (ColE1), KanR gene for bacterial selection, a marker for auxotrophic selection in yeast (e.g., a gene comprising either the URA3, LEU2, HIS3, LYS2, or MET15 ORF), recombination domains Homologous Right and Homologous Left for gene integration into either the ura3, leu2, HO, lys2, or met15 loci. The cassette includes three multiple cloning sites that each surround a GFP reporter. The first site is flanked by a pheromone inducible promoter (pFIG1) and a terminator (TDH1) sequence. An ORF encoding a pre-pro pheromone sequence is cloned into this site. The second site is flanked by a weak constitutive promoter (pPSP2) and a terminator (TDH1) sequence. In many embodiments, the same pre-pro pheromone sequence is cloned into the second site as is cloned into the first site. The context of the third site is identical to the SBE display cassette, where the insert is flanked by a signal sequence and a C terminus of a cell wall anchored protein.

FIGS. 22A and 22B show exemplary SBEs. FIG. 22A shows SBE FLAGS comprising three 3×FLAG domains (DYKDHDGDYKDHDIDYKDDDDK, SEQ ID NO: 1) joined by rigid linkers (A(EAAAK)₃A, SEQ ID NO: 2). FIG. 22B shows SBE HEMA9 comprising nine HA tags (YPYDVPDYA, SEQ ID NO: 3) organized into three domains joined by rigid linkers (A(EAAAK)₃A, SEQ ID NO: 2), where the individual domains have the form HA tag/(SG4)2/HA tag/(SG4)2/HA.

FIG. 23 shows the specificity of exemplary SBEs. The specificity of SBEs toward their cognate ligands demonstrated by agglutination assay involving antibody labeled agarose beads. Agarose beads covalently linked to anti-FLAG M2 antibodies (A2220 Sigma-Aldrich) were used as a potential agglutination partner for FLAGS CUs (sX216). Agarose beads covalently linked to anti-HA antibodies (26182 Thermo Fisher) were used as a potential agglutination partner for HEMA9 CUs (sX422). Following a clustering reaction, beads were visualized at 100× magnification using an inverted microscope. Upper left panel) anti-FLAG M2 beads clustered with sX216 cells. High level of agglutination is observed. Upper right panel) anti-HA beads clustered with sX216 cells. Negligible agglutination is observed. Lower left panel) anti-FLAG M2 beads clustered with sX422 cells. Negligible agglutination is observed. Lower right panel) anti-HA beads clustered with sX422 cells. High level of agglutination is observed.

FIG. 24 shows the dependence of signal transmission on low signal production levels. The relative density of signal producing and signal recognizing CUs was varied and reporter concentration in the supernatant was quantified using Nitrocefin-BLA analysis. The reporter strain sX128 was combined with a producer strain (sX212, sX403, sX404) at the indicated ratio of producer cells to total cells. A) Null signal production (sX128+sX212). The reporter amount decreases gradually as the number of reporter cells decreases. B) Low/medium signal production (sX128+sX403). The reporter amount peaks at the ratio 0.6. C) Medium signal production (sX128+sX404). The reporter amount peaks between the ratios of 0.6 and 0.8.

FIG. 25 shows the dependence of signal transmission on high signal production levels. The relative density of signal producing and signal recognizing CUs was varied and reporter concentration in the supernatant was quantified using Nitrocefin-BLA analysis. The reporter strain sX128 was combined with a producer strain (sX404, sX405, or sX406) at the indicated ratio of producer cells to total cells. C) Medium signal production (sX128+sX404). Same graph as in FIG. 23. D) High/medium signal production (sX128+sX405). The reporter amount peaks between the ratios 0.4 and 0.6. E) High signal production (sX128+sX406). The reporter amount peaks between the ratios 0.2 and 0.4.

FIG. 26 shows the dependence of signal transmission on signal degradation. The relative density of signal degrading CUs (strain sX437) was varied relative to the density of reporter CUs (strain sX128) and producer CUs (strain sX406). Reporter concentration in the supernatant was quantified using Nitrocefin-BLA analysis. The graph shows that even a relatively low number of signal degrading CUs, approximately 6% of signal producing CUs, is sufficient to nullify and signaling between suspended cells.

FIG. 27 shows the dependence of PIP signal strength on cluster composition in the presence of regulatory CUs (strain sX437). Evaluation of pheroimmuno propositions is demonstrated at various mixture compositions. All experiments were done in triplicates. Error bars indicate minimum and maximum values. A) Producer CUs (strain sX406) and reporter CUs (strain sX216) combined at the indicated ratio (producer:producer+reporter) and clustered with anti-FLAG M2 antibody linked beads. B) Negative control experiments lacking antibody linked beads. C) Negative control experiments where producer CUs are substituted with a null CUs (strain sX212) that secrete no pheromone.

FIG. 28 shows the dependence of PIP signal strength on cluster composition in the absence of regulatory strains. Evaluation of pheroimmuno propositions are demonstrated at various mixture compositions. All experiments were done in triplicates. Error bars indicate minimum and maximum values. D) Producer CUs (strain sX406) and reporter CUs (strain sX216) combined at the indicated ratio (producer:producer+reporter) and clustered with anti-FLAG M2 antibody linked beads. E) Negative control experiments lacking antibody linked beads. No statistically significant difference between bead containing samples and negative controls is observed indicating the need for regulatory CUs.

FIG. 29 shows evaluation of the logical operation OR in the presence of regulatory CUs (strain sX437). Producer CUs (strain sX406) and reporter CUs (strain sX216) implement a signaling cascade that transposes anti-Flag M2 antibody SBEs to reporter signal objects (hydrolyzed Nitrocefin). Producer CUs (strain sX417) and reporter CUs (strain sX422) implement a signaling cascade that transposes anti-HA antibody SBEs to the same reporter signal objects. All experiments were done in triplicates. Error bars indicate minimum and maximum values. A) CU mixture clustered with anti-FLAG M2 antibody linked beads. B) CU mixture clustered with anti-HA antibody linked beads. NC) Negative controls where antibody linked beads are absent. Chart demonstrates establishment of signaling cascades if either anti-FLAG M2 antibody linked beads OR anti-HA antibody linked beads are present.

FIG. 30 shows evaluation of the logical operation AND in the presence of regulatory CUs (strain sX437). Four CU systems are tested in recognizing combinations of three different SBEs (anti-FLAG M2 antibodies, anti-HA antibodies, SBE-X). Here SBE-X denotes any SBE with different specificity than the specificity of the named SBEs. Out of the CUs tested, only CUs of strain sX406 recognize anti-FLAG M2 antibodies and only CUs of strain sX422 recognize anti-HA antibodies. The remaining CU types recognize SBE-X. All experiments were done in triplicates. Error bars indicate minimum and maximum values. A) Reporter fold-increase generated by producer CUs (strain sX406) and reporter CUs (strain sX422) clustered with beads that are linked to both anti-FLAG M2 antibodies and anti-HA antibodies (BaHaF). B) Reporter fold-increase generated by producer CUs (strain sX406) and reporter CUs (strain sX128) clustered with the same BaHaF target objects. C) Reporter fold-increase generated by producer CUs (strain sX137) and reporter CUs (strain sX422) clustered with the same BaHaF target objects. D) Reporter fold-increase generated by producer CUs (strain sX137) and reporter CUs (strain sX1228) clustered with the same BaHaF target objects. NC) Negative controls where BaHaF beads are absent. The chart demonstrates that out of the four AND operator CU systems only the one that corresponds to the target SBEs generates a positive change in reporter signal relative to the control signal.

FIG. 31 shows evaluation of the logical operation NOT. Two CU systems are tested in recognizing combinations of three different SBEs (anti-FLAG M2 antibodies, anti-HA antibodies, SBE-X). Here SBE-X denotes any SBE with different specificity than the specificity of the named SBEs. Out of the CUs tested, only CUs of strain sX406 and sX216 recognize anti-FLAG M2 antibodies and only CUs of strain sX444 recognize anti-HA antibodies. The remaining CU types recognize SBE-X. All experiments were done in triplicates. Error bars indicate minimum and maximum values. A) Reporter foldincrease generated by producer CUs (strain sX406), reporter CUs (strain sX4216), and inhibitory CUs (sX444) clustered with beads that are linked to both anti-FLAG M2 antibodies and anti-HA antibodies (BaHaF). B) Reporter fold-increase generated by producer CUs (strain sX406), reporter CUs (strain sX216), and inhibitory CUs (sX437) clustered with the same BaHaF target objects. NC) Negative controls where BaHaF beads are absent. The chart demonstrates that if the inhibitory CUs are localized to the cluster, then no statistically significant change in reporter signal is observed relative to the control signal.

FIG. 32 shows evaluation of PIPs by PIC assay with immobilization in a 96-well plate format. Two types of CUs (strains sX384 and sX387) implement a feedback signaling network that transposes anti-Flag M2 antibody SBEs to reporter signal objects (hydrolyzed Nitrocefin). No other regulatory strains are present. Evaluation reaction takes place in an immobilizing matrix that maintains physical spacing between computing clusters. Experiments are performed in 18 replicates. Bars indicate minimum and maximum values. Negative controls include background objects but antibody linked beads are absent. The graph demonstrates the fold-change increase in readout accomplished by feedback signal correction in the cluster.

FIG. 33 shows images used to evaluate readout signals in PIC assay with immobilization. Time indicates hours passed since the start of the evaluation reaction. Eighteen replicates were evaluated. Antibody linked beads were placed in alternate rows starting with the second row of the 6×6 matrix. Negative controls are located in alternate rows starting with the first row of the 6×6 matrix. Darker shading corresponds to increased amount of hydrolyzed Nitrocefin present in the mixture.

FIG. 34 shows orthogonality for specific binding pairs of cellular CUs (hTet-CU, mTet-CU, and Azide-CU) and complexed cellular objects (TCO-Ab and DBCO-Ab).

FIG. 35 shows the stability of CU cluster formation using the original two-step method described in Example 15 (left panel) as compared to an alternate method (right panel).

FIG. 36 shows results for external signal detection by cellular CUs modified to express functional GPCR-ligand pairs (induced rsX420, rsX626, and rsX769) as measured by beta-lactamase concentration.

### DETAILED DESCRIPTION

Published biocomputing devices are ill-suited for advanced detection applications. This includes the detection of cellular targets that are defined by multivariate surface compositions. The invention herein includes systems of computing units that are engineered to have type-specific immunochemistry and to communicate by localized pheromone signals for the purpose of multivariate surface marker profile detection. The invention is used to construct immune-system-inspired biocomputers that search batch samples for the presence of target objects and produce quantifiable signals based on the search results. Due to the efficiency of the search, biocompatibility of the technology, and the achievable specificity granted by computing features, the invention lends itself to novel applications including routine screening for rare cells (e.g., circulating cancer cells, fetal cells, stem cells, pathogens, etc), quantification of cell type-specific states in heterogeneous populations (e.g., leukocyte activation, anergy, and metabolism), and high-volume detection of contaminants (e.g., foodborne pathogens and biohazards).

In one aspect, the invention is based on the observation that two earlier discoveries, programmable immunochemistry and pheromone signaling, can be synergistically composed to perform computations on surface marker compositions. This observation is important because it enables modular design of tractable devices that can be engineered using available biotechnological tools. The invention provides a class of devices and methods for analysis of batch samples and detection of target objects within the sample volumes. Devices comprise systems of computing units that operate individually in recognizing surface markers and operate jointly to integrate the information and recognize marker combinations. Recognition of surface markers is achieved through programmed immunochemistry of mixture entities. Integration of information is achieved through localized signal transmission. Devices also include computing units that regulate transmissions, e.g., degrade or amplify. Computing units are either added a priori or produced by other computing units. The behavior of computing unit may also depend on their internal states that track the unit's history.

### Definitions

Unless defined otherwise, all technical and scientific terms used herein have the same meaning as commonly understood by one of ordinary skill in the art to which the disclosure pertains. All patents, applications, published applications and other publications referenced herein are expressly incorporated by reference in their entireties unless stated otherwise. In the event that there are a plurality of definitions for a term herein, those in this section prevail unless stated otherwise.

As used herein, “a” or “an” may mean one or more than one.

“About” has its plain and ordinary meaning when read in light of the specification, and may be used, for example, when referring to a measurable value and may be meant to encompass variations of ±20% or ±10%, more preferably ±5%, even more preferably ±1%, and still more preferably ±0.1% from the specified value.

### Biological Computing Systems

The invention provides compositions comprising collected and engineered objects (entities that have informational significance) and media (all other entities). Objects are delimited by their surfaces (e.g., cellular membranes, cells walls, distal molecular domains) and are suspended or submerged in the media (e.g. liquid or gas). Hence, objects may be individual entities or clusters formed through aggregation of entities (e.g., covalent or non-covalent bonding). Objects may be classified by the types of surface-bound entities (SBEs) they expose to the medium. Each SBE is typed by how it is recognized (e.g., by van der Waals forces, hydrogen bonds, hydrophobic and/or ionic interactions) by each SBE type. Presentation of SBEs may be either constant, spontaneous, or induced (e.g., initiated following detection of signals transmitted in the medium or following direct interaction with objects). Objects are classified at each point in time. An object's class may change in time as the object's SBE profile changes. An object is at a given point in time called a target object if its current SBE profile belongs to one of the predetermined classes. If two target objects have statistically similar SBE profiles, they are assigned to the same target object class.

In one aspect, provided herein is a system for biological computing, comprising one or more (such as any of 2, 3, 4, 5, or more) computing units (CUs), wherein the one or more CUs are configured to interact with one or more (such as any of 2, 3, 4, 5, or more) sample objects derived from an input sample such that an output signal indicative of a characteristic of the input sample is generated. In some embodiments, the system further comprises the one or more sample objects. In some embodiments, the system is configured such that one or more computational clusters can be formed, wherein each computational cluster comprises, independently, one or more of the CUs in the system. In some embodiments, one or more of the computational clusters further comprise, independently, one or more of the sample objects. In some embodiments, the output signal comprises a level of an output signal object (SO) in a medium comprising the system. In some embodiments, the input sample is a biological sample derived from an individual. In some embodiments, the input sample is an environmental sample. In some embodiments, the input sample is a synthetic sample.

### Computing Units (CUs)

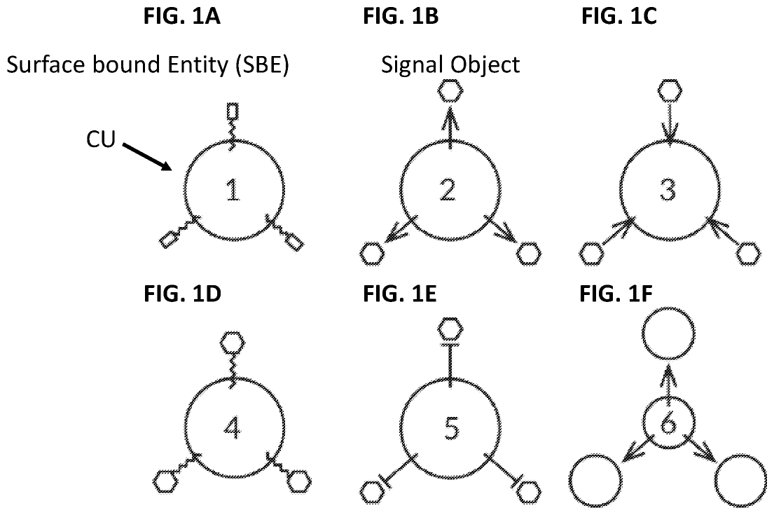

A computing unit (CU) is an object that has the potential to affect the informational content of the system. Informational content may be affected in a number of different ways as is illustrated in FIGS. 1A-1F. A CU may be an object with a classified SBE profile (FIG. 1A) and thereby may mediate object association. A CU may be an object that produces other CUs or signal objects (FIGS. 1B and 1F) and thereby may affect the contents of the system. A CU may be an object that recognizes signal objects (FIG. 1C) and thereby may affect the memory capacity of the system.

In some embodiments, a CU is monomeric or multimeric molecule (e.g., a polypeptide, polypeptide derivative, nucleic acid). In other embodiments, a CU is a wildtype cell (e.g., of bacterial, yeast, mammalian origin) or a synthetic cell (e.g., any genetically engineered cell or liposome). In particular, a CU may be a synthetic cell based on or derived from a yeast cell. Suitable yeast include, but are not limited to: Pichia pastoris, Saccharomyces cerevisiae, Arxula adeninivorans (Blastoborys adeninivorans), Candida boidinii, Hansenual polymorpha (Pichia angusta), Kluveromyces lactis, Yarrowia lipolytica, etc.

A computing object may include non-native elements, native elements in non-native locations, or other alterations to native elements. Introduced elements include, but are not limited to, signal object generators, SBEs, reporter molecules, regulatory sequences, genetic selection markers, other types of non-genetic markers (e.g., magnetic, immunological), reference reporter molecules, enzyme coding genes (e.g., protease, kinase, phosphatase), etc. In various embodiments, modifications are performed by introducing genetic changes at select chromosomal regions. Chromosomal modifications can either introduce new genetic elements at select loci (e.g., URA3, LEU2, HO, MET15, LYS3, etc.) or modify native elements (promoters, degradation tags, termini tags, transposon sites, etc.) at native loci. In some embodiments, modifications are performed by introducing additional genetic material, e.g., plasmids or synthetic chromosomes.

In some embodiments, according to any of the systems for biological computing described herein, each of the one or more CUs in the system is, independently, i) associated with one or more surface-bound entities (SBEs); ii) capable of recognizing an SO; iii) capable of producing an SO; iv) capable of degrading an SO; v) capable of producing a change in a material property of a medium comprising the system; and/or vi) capable of producing another CU. In some embodiments, each of the one or more CUs comprises, independently, a cell or a molecule.

In some embodiments, according to any of the systems described herein comprising a CU capable of producing a change in a material property of a medium comprising the system, the material property of the medium is an optical property, an electrical property, or a thermal property.

In some embodiments, according to any of the systems described herein comprising a first CU capable of producing a second CU, the second CU is of the same type as the first CU or is of a different type than the given CU.

In some embodiments, according to any of the systems described herein comprising a first CU and a second CU, the first CU is associated with a first SBE and the second CU is associated with a second SBE. In some embodiments, the first and second SBEs are capable of forming a complex comprising the first and second SBEs.

In some embodiments, according to any of the systems described herein, the system comprises a CU comprising (or consisting of) a cell, also referred to herein as a “cellular CU.” In some embodiments, the cell is a wild-type cell (e.g., a wild-type bacterial, yeast, or mammalian cell). In some embodiments, the cell is an engineered or synthetic cell (e.g., an engineered yeast cell). In some embodiments, the engineered cell is an engineered yeast cell derived from Pichia pastoris, Saccharomyces cerevisiae, Arxula adeninivorans (Blastoborys adeninivorans), Candida boidinii, Hansenual polymorpha (Pichia angusta), Kluveromyces lactis, Yarrowia lipolytica, and the like. In some embodiments, the SBE is a surface-bound molecule present on the cell (e.g., naturally present on the cell). In some embodiments, the CU comprises a cell comprising an SBE. In some embodiments, the SBE is covalently attached to an anchor present on the cell. In some embodiments, the SBE is covalently attached to the anchor by a linker. In some embodiments, the linker comprises a repeat motif. In some embodiments, the linker facilitates accessibility of the SBE and/or increases an effective contact area between a sample object and the CU in a medium comprising the system.

In some embodiments, according to any of the cellular CUs described herein, the cellular CU comprises an SBE, wherein the SBE is a modified form of a surface entity present on the cell of the cellular CU. In some embodiments, the SBE is a modified form of a glycophosphatidylinositol (GPI)-anchored protein of the cell. In some embodiments, the surface entity is modified by introduction of a chemical conjugation moiety (e.g., a tetrazine compound, an azide compound, and the like) and/or a hydrophilic modulator (e.g., polyethylene glycol (PEG)-based compounds, including monodisperse, monofunctional, homobifunctional, heterobifunctional, and multi-arm compounds, such as PEG-N-hydroxysuccinimide (NHS) esters (e.g., PEG-succinimidyl carboxymethyl (SCM) esters), and the like). In some embodiments, the SBE is modified by introduction of a chemical conjugation moiety. In some embodiments, the SBE is modified by introduction of a chemical conjugation moiety and the cellular CU further comprises another SBE modified by introduction of a hydrophilic modulator. In some embodiments, the cell of the cellular CU is a yeast cell, such as an engineered yeast cell.

In some embodiments, according to any of the system elements (e.g., CUs) described herein modified by introduction of a chemical conjugation moiety, the chemical conjugation moiety includes, without limitation, (i) moieties useful for strain-promoted alkyne-azide cycloaddition (SPAAC), such as cyclooctyne (OCT), monofluorinated cyclooctyne (MOFO), difluorocyclooctyne (DIFO), dimethoxyazacyclooctyne (DIMAC), carboxymethylmonobenzocyclooctyne (COMBO), dibenzocyclooctyne (DIBO), dibenzoazacyclooctyne (DIBAC), biarylazacyclooctynone (BARAC), bicyclononyne (BCN), 3,3,6,6-tetramethylthiaheptyne (TMTH), 2,3,6,7-tetramethoxy-DIBO (TMDIBO), sulfonylated DIBO (S-DIBO), pyrrolocyclooctyne (PYRROC), and azide derivatives (see, e.g., Dommerholt, J., et al. (2016). Cycloadditions in Bioorthogonal Chemistry (pp. 57-76). Springer, Cham); (ii) moieties useful for strain-promoted alkyne-nitrone cycloaddition (SPANC), such as OCT, MOFO, DIFO, DIMAC, COMBO, DIBO, DIBAC, BARAC, BCN, TMTH, and nitrone derivates; (iii) moieties useful for photoinduced 1,3-dipolar cycloaddition, such as tetrazole and alkene derivatives; (iv) moieties useful for copper(I)-catalyzed azide alkyne cycloaddition (CuAAC), such as azide and alkyne derivatives; (v) moieties useful for inverse electron-demand Diels-Alder (IEDDA) cycloaddition, such as transcyclooctene (TCO) and tetrazine derivatives; (vi) biotin and avidin/streptavidin derivatives; (vii) SpyTag and SpyCatcher derivatives (see, e.g., Zakeri, B., et al. (2012). Proceedings of the National Academy of Sciences, 109(12), E690-E697); (viii) self-labeling proteins, such as human alkylguanine-DNA alkyltransferases (AGT) (e.g., SNAP-tag and CLIP-tag) and bacterial haloalkane dehalogenase enzymes (e.g., Halo-tag); and (ix) benzylguanine/benzylcytosine derivatives.

For example, in some embodiments, according to any of the cellular CUs described herein, the cellular CU comprises a yeast cell (e.g., an engineered yeast cell) comprising an SBE, wherein the SBE is a GPI-anchored protein of the yeast cell modified by introduction of a chemical conjugation moiety (e.g., a tetrazine compound, an azide compound, and the like) and/or a hydrophilic modulator (e.g., a PEG-NHS ester and the like). In some embodiments, the GPI-anchored protein is modified by introduction of a tetrazine compound, optionally wherein the GPI-anchored protein is further modified by introduction of a hydrophilic modulator, such as a PEG-NHS ester. In some embodiments, the GPI-anchored protein is modified by introduction of a tetrazine compound, and the cellular CU further comprises another SBE modified by introduction of a hydrophilic modulator. In some embodiments, the tetrazine compound is tetrazine or methyltetrazine. Tetrazine compounds can be conjugated to GPI-anchored proteins by any method known in the art useful for such purposes, for example by using a reagent containing the tetrazine compound fused to a PEG-NHS ester. In some embodiments, the GPI-anchored protein is modified by introduction of an azide compound, optionally wherein the GPI-anchored protein is further modified by introduction of a hydrophilic modulator, such as a PEG-NHS ester. In some embodiments, the GPI-anchored protein is modified by introduction of an azide compound, and the cellular CU further comprises another SBE modified by introduction of a hydrophilic modulator. Azide compounds can be conjugated to GPI-anchored proteins by any method known in the art useful for such purposes, for example by using a reagent containing the azide compound fused to a PEG-NHS ester.

In some embodiments, according to any of the systems described herein, the system comprises a CU comprising (or consisting of) a molecule (e.g., a monomeric or multimeric molecule), also referred to herein as a “molecular CU.” In some embodiments, the molecule includes, without limitation, a polypeptide, a polypeptide derivative, a nucleic acid, and a solid support. In some embodiments, the CU comprises a polypeptide. In some embodiments, the polypeptide is an antibody, e.g., a bispecific antibody (BsAb). In some embodiments, the polypeptide is an enzyme. In some embodiments, the system further comprises an agent, and the enzyme is capable of converting the agent into an SO. In some embodiments, the SO is an internal SO capable of being recognized by a) at least one CU in the system and/or b) at least one sample object in the system. In some embodiments, the SO is an output SO, and the output signal comprises a level of the SO. In some embodiments, the CU comprises a solid support. In some embodiments, the solid support is a functionalized bead. In some embodiments, the molecular CU is complexed (i.e., non-covalently bound) to a cell, such as a cellular object in a sample.

In some embodiments, according to any of the molecular CUs described herein, the molecular CU comprises a molecule covalently or non-covalently linked to an amine group. In some embodiments, the molecule is modified to introduce a functional group selected from a binding moiety (e.g., a trans-cyclooctene (TCO) moiety for binding to tetrazine compounds, a dibenzocyclooctyne (DBCO) moiety for binding to azide compounds, and the like), a hydrophilic modulator, a hydrophobic modulator, a fluorescent probe, a semiconductor particle, a hydrogel adaptor, and the like. For example, in some embodiments, the molecule is modified by chemical conjugation with a binding moiety selected from a trans-cyclooctene (TCO) moiety, a dibenzocyclooctyne (DBCO) moiety, and the like. In some embodiments, the binding moiety is conjugated to the molecule via a linker, such as a PEG linker (e.g., a PEG-NHS linker and the like). In some embodiments, the molecular CU comprises an immunoglobulin (e.g., an IgG, such as an anti-bovine serum albumin (BSA) antibody). In some embodiments, the immunoglobulin has a first specificity, and is modified to introduce a second specificity by conjugation with a binding moiety. In some embodiments, the molecular CU is complexed to a cell, such as a cellular object in a sample.

For example, in some embodiments, according to any of the molecular CUs described herein, the molecular CU comprises an immunoglobulin (e.g., an IgG, such as an anti-BSA antibody) modified by introduction of a binding moiety. In some embodiments, the binding moiety is selected from a TCO moiety, a DBCO moiety, and the like. In some embodiments, the binding moiety is conjugated to the immunoglobulin via a linker, such as a PEG linker. In some embodiments, the linker is selected from a PEG-NHS linker and the like. In some embodiments, the molecular CU comprises an immunoglobulin modified by introduction of a TCO moiety via a PEG linker. TCO moieties can be conjugated to immunoglobulins using any method known in the art useful for such purposes, for example by using a reagent containing the TCO moiety fused to a PEG linker (e.g., a PEG-NHS linker and the like). In some embodiments, the molecular CU comprises an immunoglobulin modified by introduction of a DBCO moiety via a PEG linker. DBCO moieties can be conjugated to immunoglobulins using any method known in the art useful for such purposes, for example by using a reagent containing the DBCO moiety fused to a PEG linker (e.g., a PEG-NHS linker and the like). In some embodiments, the molecular CU is complexed to a cell, such as a cellular object in a sample.

In some embodiments, according to any of the cellular CUs described herein, the cellular CU i) comprises a heterologous SBE capable of intracellular signaling (e.g., a heterologously expressed surface receptor, such as a G-protein coupled receptor (GPCR)) and/or ii) is capable of expressing an SO (e.g., an SO capable of being recognized as an SO produced by a cell in a sample in which the cellular CU is present, such as a GPCR ligand). In some embodiments, the heterologous SBE is capable of transmitting an intracellular signal in response to binding an SO in the system, such as an SO produced by the cellular CU and/or an SO produced by a cell in a sample in which the cellular CU is present. In some embodiments, the cellular CU comprises the heterologous SBE and is capable of expressing an SO capable of activating the heterologous SBE to transmit an intracellular signal. In some embodiments, the cellular CU comprises a heterologous GPCR (e.g., somatostatin (SST) receptor 2 (SSTR2), neurotensin (NTS) receptor 1 (NTSR1), or complement C5a receptor 1 (C5AR1)) and is capable of expressing a ligand of the GPCR (e.g., SST-14, NTS, or C5a). The presence and/or level of the SO in a sample comprising the cellular CU can be determined by detecting, directly or indirectly, a product of the intracellular signaling. For example, where the heterologous SBE is a GPCR such as SSTR2, NTSR1, or C5AR1, the presence and/or level of an SO comprising a ligand of the GPCR in the sample can be determined by measuring the beta-lactamase concentration in the sample, where increasing amounts of beta-lactamase correspond to increased activation of the heterologous GPCR by the ligand. In some embodiments, the cell of the cellular CU is a yeast cell, such as an engineered yeast cell.

For example, in some embodiments, according to any of the cellular CUs described herein, the cellular CU comprises a yeast cell (e.g., an engineered yeast cell) i) comprising a heterologous GPCR and ii) capable of expressing a ligand of the heterologous GPCR. In some embodiments, the heterologous GPCR is selected from SSTR2, NTSR1, and C5AR1. In some embodiments, the yeast cell is engineered to constitutively express the heterologous GPCR. In some embodiments, when the heterologous GPCR is SSTR2, NTSR1, or C5AR1, the GPCR ligand is SST-14, NTS, or C5a, respectively. In some embodiments, the yeast cell is engineered to inducibly secrete the GPCR ligand. For example, in some embodiments, the yeast cell is engineered to place a nucleic acid encoding the GPCR ligand under the control of an inducible promoter. Inducible promoter systems are known in the art (e.g., tetracycline-on systems) and can be selected as necessary based on the computing system configuration and requirements. The presence and/or level of a ligand of the heterologous GPCR in a sample comprising the cellular CU can be determined by detecting, directly or indirectly, a product of the GPCR signaling, e.g., by measuring the beta-lactamase concentration in the sample.

### Sample Objects

The systems described herein may comprise sample objects from one or more samples, including e.g., samples of biological, environmental, and/or synthetic origin. The biological portions of the sample can be compositions of biological fluids (e.g., whole blood, serum, plasma, sputum, urine, saliva, nipple aspirate, ductal lavage, vaginal fluid, nasal fluid, ear fluid, gastric fluid, cerebrospinal fluid, sweat, pericrevicular fluid, semen, prostatic fluid, feces, cell lysate, tears), tissue samples (e.g., hair, skin, biopsy material), and enriched biological material (e.g., various cell types, exosomes). The environmental portions of the sample can be compositions of soil, dirt, sewage, air, water, plant material, etc. Manmade portions of the samples may include any synthesized materials, e.g., forensic, biosafety screening samples. The remaining system objects comprise computing elements that act as signal objects and/or computing units (CUs).

In some embodiments, according to any of the systems for biological computing described herein, each of the one or more sample objects in the system is, independently, i) associated with one or more SBEs; ii) capable of recognizing an SO; iii) capable of producing an SO; iv) capable of degrading an SO; v) capable of producing a change in a material property of a medium comprising the system; and/or vi) capable of producing another sample object. In some embodiments, each of the one or more sample objects comprises, independently, a cell or a molecule.

In some embodiments, according to any of the systems described herein, the system comprises a sample object comprising (or consisting of) a cell. In some embodiments, the sample object comprises a cell associated with an SBE. In some embodiments, the SBE is an antigen on the cell surface, such as a cell surface receptor. In some embodiments, the antigen is a disease-associated antigen, such as a cancer-associated antigen. In some embodiments, the SBE is a marker indicating a state of the sample object, including, for example, differentiation factors and clusters of differentiation. In some embodiments, the SBE is a biological signal, including, for example, MHC, MHC epitope complex, and glycocalyx. In some embodiments, the SBE is a marker indicating an activity of the sample object, including, for example, receptors, receptor ligand complexes, ion channels, and their modified forms. In some embodiments, the SBE is a pathogenic marker, including, for example, glycoproteins and lectins. In some embodiments, the SBE is a synthetically produced molecule, including, for example, surface tags, displayed epitopes, and conjugated molecules.

### Computational Clusters

In some embodiments, according to any of the systems described herein, the system comprises one or more (such as 2, 3, 4, 5, or more) CUs capable of forming a cluster, also referred to herein as a computational cluster, in a medium comprising the system. In some embodiments, the computational cluster further comprises one or more (such as 2, 3, 4, 5, or more) sample objects. In some embodiments, the system comprises a computational cluster comprising the one or more CUs, and optionally one or more sample objects.

In some embodiments, according to any of the systems described herein comprising a CU and a sample object capable of forming a cluster, the CU is configured such that it can associate with the sample object by diffusion of the CU and the sample object in a medium comprising the system.

In some embodiments, according to any of the systems described herein comprising a CU and a sample object capable of forming a cluster, the CU is configured such that it can associate with the sample object by application of a force to a medium comprising the system such that the CU and the sample object are placed in proximity to one another. In some embodiments, the force is a centrifugal force. In some embodiments, the force is a magnetic force. In some embodiments, the force is an electrostatic force.

In some embodiments, according to any of the systems described herein comprising a cluster comprising a CU, the CU is configured to respond to an SO present in a boundary layer around the cluster by producing another SO that is not restricted to the boundary layer (see, for example, FIG. 16).

In some embodiments, according to any of the systems described herein comprising a cluster comprising a first CU, the system further comprises a second CU that is not localized to the cluster, wherein the second CU is configured to degrade an SO produced by the first CU (see, for example, FIG. 17).

Exemplary computational clusters include those formed by association of orthogonal pairs of cellular CUs and molecular CUs as described herein. In some embodiments, an orthogonal pair of a cellular CU and a molecular CU comprises i) a cellular CU comprising an SBE, wherein the SBE is a surface entity present on the cell of the cellular CU modified by introduction of a ligand; and ii) a molecular CU comprising a molecule covalently or non-covalently linked to an amine group modified by introduction of a ligand-binding moiety capably of binding to the ligand of i). In some embodiments, the cellular CU of i) comprises a yeast cell (e.g., an engineered yeast cell) comprising a GPI-anchored protein modified by introduction of a ligand comprising a tetrazine compound or an azide compound, optionally wherein the GPI-anchored protein is further modified by introduction of a hydrophilic modulator (e.g., a PEG-NHS ester and the like), and the molecular CU of ii) comprises an immunoglobulin (e.g., an IgG, such as an anti-BSA antibody) modified by introduction of a ligand-binding moiety comprising a) a TCO moiety if the ligand comprises a tetrazine compound, or b) a DBCO moiety if the ligand comprises an azide compound. In some embodiments, the ligand comprises methyltetrazine and the ligand-binding moiety comprises a TCO moiety. In some embodiments, the ligand comprises an azido group and the ligand-binding moiety comprises a DBCO moiety. In some embodiments, the cellular CU of i) further comprises another SBE modified by introduction of a hydrophilic modulator. In some embodiments, the immunoglobulin is an IgG antibody. In some embodiments, the antibody is an anti-BSA antibody. It is to be appreciated that the class and specificity of the immunoglobulin can be selected as necessary based on the computing system configuration and requirements.

In some embodiments, according to any of the computational clusters described herein, the computational cluster comprises i) a cellular CU comprising a yeast cell (e.g., an engineered yeast cell) comprising a GPI-anchored protein modified by introduction of a ligand comprising a tetrazine compound, optionally wherein the GPI-anchored protein is further modified by introduction of a hydrophilic modulator (e.g., a PEG-NHS ester and the like); and ii) a molecular CU comprising an immunoglobulin (e.g., an IgG, such as an anti-BSA antibody) modified by introduction of a ligand-binding moiety comprising a TCO moiety. In some embodiments, the ligand comprises methyltetrazine. In some embodiments, the cellular CU of i) further comprises another SBE modified by introduction of a hydrophilic modulator. In some embodiments, the immunoglobulin is an IgG antibody. In some embodiments, the antibody is an anti-BSA antibody. It is to be appreciated that the class and specificity of the immunoglobulin can be selected as necessary based on the computing system configuration and requirements. In some embodiments, the computational cluster is more stable as compared to a corresponding computational cluster where a) the tetrazine compound of the ligand is replaced with an azide compound, or b) the TCO moiety of the ligand-binding moiety is replaced with a DBCO moiety. Cluster stability can be measured using techniques known in the art useful for such purposes, such as by determining resistance of the cluster to agitation-mediated disassembly. In some embodiments, a computational cluster cannot be formed if a) the tetrazine compound of the ligand is replaced with an azide compound, or b) the TCO moiety of the ligand-binding moiety is replaced with a DBCO moiety.

In some embodiments, according to any of the computational clusters described herein, the computational cluster comprises i) a cellular CU comprising a yeast cell (e.g., an engineered yeast cell) comprising a GPI-anchored protein modified by introduction of a ligand comprising an azide compound, optionally wherein the GPI-anchored protein is further modified by introduction of a hydrophilic modulator (e.g., a PEG-NHS ester and the like); and ii) a molecular CU comprising an immunoglobulin (e.g., an IgG, such as an anti-BSA antibody) modified by introduction of a ligand-binding moiety comprising a DBCO moiety. In some embodiments, the cellular CU of i) further comprises another SBE modified by introduction of a hydrophilic modulator. In some embodiments, the immunoglobulin is an IgG antibody. In some embodiments, the antibody is an anti-BSA antibody. It is to be appreciated that the class and specificity of the immunoglobulin can be selected as necessary based on the computing system configuration and requirements. In some embodiments, the computational cluster is more stable as compared to a corresponding computational cluster where a) the azide compound of the ligand is replaced with a tetrazine compound, or b) the DBCO moiety of the ligand-binding moiety is replaced with a TCO moiety. In some embodiments, a computational cluster cannot be formed if a) the azide compound of the ligand is replaced with a tetrazine compound, or b) the DBCO moiety of the ligand-binding moiety is replaced with a TCO moiety.

In one aspect, provided herein is a method of preparing a computational cluster, the method comprising i) a first step comprising complexing cellular objects in a sample with a molecular CU as described herein comprising a ligand-binding moiety (e.g., an antigen-specific IgG modified by introduction of the ligand-binding moiety, such as a TCO or DBCO moiety) to generate cellular object/molecular CU complexes; and ii) a second step comprising mechanically engaging (such as by application of a centrifugal force, a magnetic force, or an electrostatic force) the cellular object/molecular CU complexes with an orthogonal cellular CU as described herein presenting a ligand capable of being bound by the ligand-binding moiety of the molecular CU (e.g., a yeast cell comprising a GPI-anchored protein modified by introduction of the ligand), thereby forming a computational cluster.

In some embodiments, provided herein is a method of preparing a computational cluster, the method comprising i) a first step comprising complexing cellular objects in a sample with a molecular CU as described herein comprising an immunoglobulin modified by introduction of a ligand-binding moiety comprising a TCO moiety to generate cellular object/molecular CU complexes; and ii) a second step comprising mechanically engaging (such as by application of a centrifugal force, a magnetic force, or an electrostatic force) the cellular object/molecular CU complexes with an orthogonal cellular CU as described herein comprising a yeast cell comprising a GPI-anchored protein modified by introduction of a ligand comprising a tetracycline compound, thereby forming a computational cluster. In some embodiments, the computational cluster is more stable as compared to a corresponding computational cluster where a) the tetrazine compound of the ligand is replaced with an azide compound, or b) the TCO moiety of the ligand-binding moiety is replaced with a DBCO moiety. In some embodiments, a computational cluster cannot be formed if a) the tetrazine compound of the ligand is replaced with an azide compound, or b) the TCO moiety of the ligand-binding moiety is replaced with a DBCO moiety. In some embodiments, the computational cluster is more stable as compared to a corresponding computational cluster prepared by an alternate method comprising i) a first step comprising covalently linking a corresponding cellular CU with an antigen-specific immunoglobulin, and ii) a second step comprising mechanically engaging (such as by application of a centrifugal force, a magnetic force, or an electrostatic force) the modified cellular CU with antigen presenting cellular objects.

In some embodiments, provided herein is a method of preparing a computational cluster, the method comprising i) a first step comprising complexing cellular objects in a sample with a molecular CU as described herein comprising an immunoglobulin modified by introduction of a ligand-binding moiety comprising a DBCO moiety to generate cellular object/molecular CU complexes; and ii) a second step comprising mechanically engaging (such as by application of a centrifugal force, a magnetic force, or an electrostatic force) the cellular object/molecular CU complexes with an orthogonal cellular CU as described herein comprising a yeast cell comprising a GPI-anchored protein modified by introduction of a ligand comprising an azide compound, thereby forming a computational cluster. In some embodiments, the computational cluster is more stable as compared to a corresponding computational cluster where a) the azide compound of the ligand is replaced with a tetrazine compound, or b) the DBCO moiety of the ligand-binding moiety is replaced with a TCO moiety. In some embodiments, a computational cluster cannot be formed if a) the azide compound of the ligand is replaced with a tetrazine compound, or b) the DBCO moiety of the ligand-binding moiety is replaced with a TCO moiety. In some embodiments, the computational cluster is more stable as compared to a corresponding computational cluster prepared by an alternate method comprising i) a first step comprising covalently linking a corresponding cellular CU with an antigen-specific immunoglobulin, and ii) a second step comprising mechanically engaging (such as by application of a centrifugal force, a magnetic force, or an electrostatic force) the modified cellular CU with antigen presenting cellular objects.

In another aspect, provided herein is a method of preparing a computational cluster, the method comprising i) a first step comprising covalently linking a cellular CU as described herein with an antigen-specific immunoglobulin, and ii) a second step comprising mechanically engaging (such as by application of a centrifugal force, a magnetic force, or an electrostatic force) the modified cellular CU with an antigen presenting sample object (e.g., a cellular sample object presenting an antigen capable of being bound by the immunoglobulin), thereby forming a computational cluster.

### Surface-Bound Entities (SBEs)

SBEs mediate object association either directly (see FIG. 2A) or through intermediate objects (see FIG. 2B). In some embodiments, an SBE profile defines the CU class that subsequently serves in constructing systems that perform particular computing functions. The SBE profile may be constant in time, change spontaneously (e.g., by random change of internal state), change as a result of an internal state change (e.g., caused by an internal engineered mechanism), or change as a result of induction (e.g., by chemical, temperature, light, or electromagnetic changes).

In some embodiments, a CU is a molecular entity (molecular CU) with a single SBE or multiple SBE moieties of the same or different specificities. This includes the collection of immunoglobulins, their derivatives (e.g., scFv, Fab, diabody, etc.), or similar binding entities with some level of molecular specificity (e.g., DARPins, TALENS, antigens, nucleic acids).

In some embodiments, a CU is a cell (cellular CU) displaying SBEs on its outermost surface (e.g. a cell wall or membrane). In this case, the SBEs and the cell may be produced separately and subsequently associated by standard practices, e.g., through immunological labeling. In some embodiments, an SBE is tethered to the cellular CU surface by interaction with surface anchored proteins. In some embodiments, an SBE is anchored to the cellular CU surface as a fusion polypeptide with a surface anchor moiety that interacts either with an outer membrane of the cellular CU (e.g., by hydrophobic interaction with membrane lipids) or a cell wall of the cellular CU (e.g., covalent bonding to cell wall polysaccharides or polypeptides). Alternatively, SBEs may be produced by the CU itself and tethered or anchored on the surface. For example, an SBE of a cellular CU can include, without limitation, integral membrane proteins, such as surface receptors (e.g., GPCRs and the like).

Engineered display of SBEs in various cellular systems has been disclosed in a number of publications. Methods address a wide range of organisms ranging from phage and E. coli to yeast and general eukaryotic systems. Methods address protein folding, secretion, surface capture, and anchoring or tethering mechanisms. In these works, the applications and design methods are concerned with coopting or improving molecular functions. For example, surface display combined with standard sorting or enrichment methods can be used for optimization of antibodies or antibody fragments. More examples include molecular analysis of displayed entities and biocatalysis by enzyme display. Display of SBEs for efficient and specific interaction with other surfaces is not addressed.

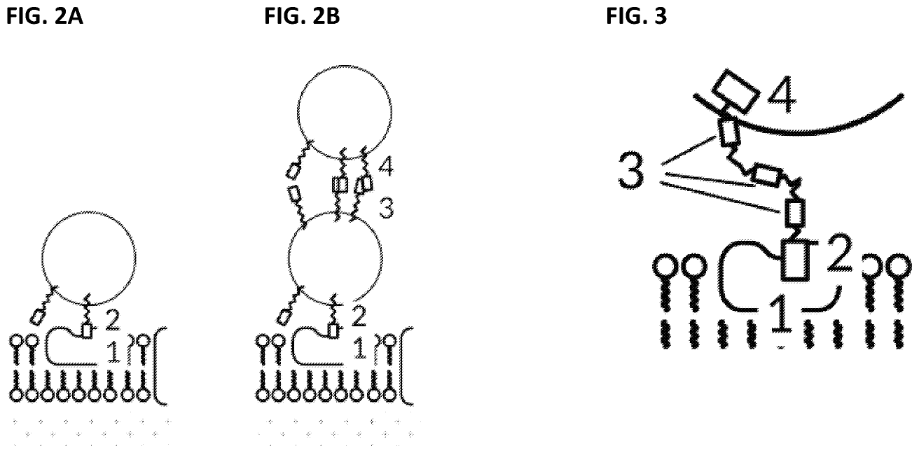

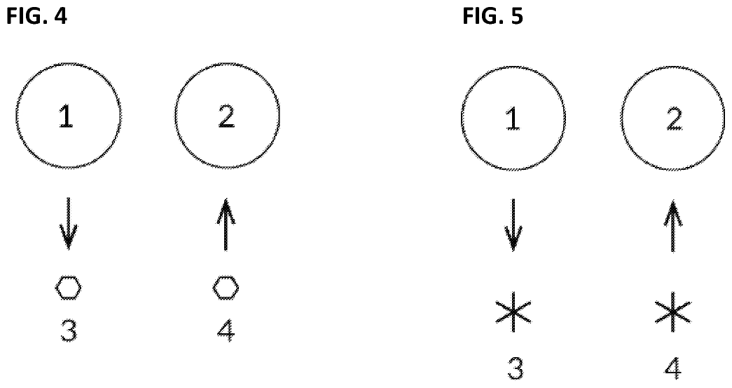

In some embodiments, SBEs are displayed as part of glycosylphosphatidylinositol (GPI)-anchored protein fusions (see FIG. 3). The recombinant structure of the fusion protein is optimized to promote interaction between system objects and, in particular, between cellular CUs and target objects. In some cases, the constructs include domains of common yeast flocculation proteins, e.g., S. cerevisiae Flo1, Flo5, Flo9, Flo10 and Flo11. The retained domains may include putative GPI associated moieties. The retained domains may also include truncated fragments of the extracellular domains the purpose of which is to increase the area of contact between adjoining objects and thereby increase the strength of association between objects displaying complementary SBEs.

In some embodiments, according to any of the systems described herein comprising a CU associated with an SBE, the SBE is capable of binding to a cognate binding partner. In some embodiments, the cognate binding partner is an SO. In some embodiments, the SO is an internal SO. In some embodiments, the SO is an external SO. In some embodiments, the SO is produced by a CU in the system. In some embodiments, the SO is produced by a sample object in the system. In some embodiments, the CU is capable of producing the SO. In some embodiments, the CU is capable of degrading the SO. In some embodiments, binding of the SBE to the SO modulates an activity of the CU. In some embodiments, binding of the SBE to the SO modulates the ability of the CU to produce an SO. In some embodiments, binding of the SBE to the SO modulates the ability of the CU to degrade an SO. In some embodiments, the cognate binding partner is another SBE. In some embodiments, binding of the SBE to the other SBE modulates an activity of the CU. In some embodiments, binding of the SBE to the other SBE modulates the ability of the CU to produce an SO. In some embodiments, binding of the SBE to the other SBE modulates the ability of the CU to degrade an SO. In some embodiments, the SBE comprises an SO.

In some embodiments, according to any of the systems described herein comprising a CU associated with an SBE, the SBE is covalently attached to a surface of the CU. In some embodiments, the SBE is covalently attached to the surface of the given CU by a linker. In some embodiments, the linker comprises a repeat motif. In some embodiments, the linker is a polypeptide linker. In some embodiments, the CU is a cell, and the SBE is covalently attached to an antigen present on the surface of the cell. For example, in some embodiments, the SBE is a polypeptide covalently attached to a surface receptor of a cell. In some embodiments, the CU is a solid support, and the SBE is covalently attached to a surface of the solid support. For example, in some embodiments, the SBE (e.g., a polypeptide SBE) is covalently attached to a surface of a bead (e.g., a functionalized bead).

In some embodiments, according to any of the systems described herein comprising a CU associated with an SBE, the SBE is non-covalently attached to a surface of the CU. In some embodiments, the SBE comprises a binding moiety that binds to a surface of the CU. In some embodiments, the CU is a cell, and the SBE comprises a binding moiety that binds to an antigen present on the surface of the cell. For example, in some embodiments, the SBE comprises a ligand that binds to a surface receptor of a cell. In some embodiments, the SBE binding moiety is an antibody moiety. In some embodiments, the CU is a solid support, and the SBE comprises a binding moiety that binds to a surface of the solid support. For example, in some embodiments, the SBE (e.g, a polypeptide SBE) comprises a binding moiety that binds to a surface of a bead (e.g., a functionalized bead).

In some embodiments, according to any of the systems described herein comprising a sample object associated with an SBE, the SBE is capable of binding to a cognate binding partner. In some embodiments, the cognate binding partner is an SO or another SBE. In some embodiments, the SBE comprises an SO.

In some embodiments, according to any of the systems described herein comprising a sample object associated with an SBE, the SBE is covalently attached to a surface of the sample object.

In some embodiments, according to any of the systems described herein comprising a sample object associated with an SBE, the SBE is non-covalently attached to a surface of the sample object.

### Signal Modulation

In some embodiments, a CU affects internal or external signals. A CU may affect a single or multiple signals directly (e.g., by producing, degrading, or transforming signals) or indirectly (e.g., by producing CUs) (see FIGS. 1B-1F). Effects on signals may be constant or time-varying, where changes may occur spontaneously, following a change in internal state, or during signal induction. Signals can be affected while at least partially exposed to the medium. In some embodiments, a CU catalyzes processing of signals. Such a catalyst may be an enzyme that nullifies signal objects by affecting their degradation (see FIG. 1E) or an enzyme that changes the specificity of the signal object towards SBEs. For example, an inactive signal object may be split into one or more active signal objects recognized by specific SBEs. In the case of peptide signals or their derivatives, the enzyme may be a protease that either specifically or nonspecifically recognizes and cleaves the signal amino acid sequence. Purified proteases may be added to a medium comprising the system in an active or inactive form that is later cis or trans activated. A wide variety of commercially available proteases may be used for this purpose. Heterologous, native, or engineered proteases may also be secreted by system CUs (e.g., bacterial proteases from the subtilisin family or yeast proteases from the barrierpepsin family).

In some embodiments, CUs affect signals directly by secreting signal objects into the medium. In cases involving cellular CUs, signal objects and other CUs may be secreted and either linked to the CU surface (see FIG. 1D) or released into the medium (see FIG. 1B). Such secretion may be constant in time, induced by recognized signals, or induced following an internal state change. Intracellular objects may be loaded in the CU any time prior to use (e.g., by electroporation or other disclosed methods for peptide transfection), however, in most cases, intracellular objects are synthesized by the CU using available or engineered metabolic processes. Signal objects may be produced from available metabolites by appropriate synthesizing enzymes (e.g., autoinducer synthases). Polypeptide objects may be produced by either heterologous or homologous gene expression, where the expression itself may be either constant in time, or time-varying with changes occurring either upon signal induction or internal state change. Moreover, upon synthesis, signal objects or potential CUs may be stored by the current CU for some period of time prior to secretion or secreted directly. Storage of objects and their conditional secretion may be accomplished by synthesis of object precursors and rapid secretion following final processing (e.g., addition of functional groups or release by cleavage of pre domains), where the processing itself may be induced through regulated gene expression or posttranslational activation of catalyzing agents.

In some embodiments, according to any of the secreted signal objects described herein, the secreted signal object is a yeast pheromone. Expression of yeast pheromones may then rely on the presence of either wildtype coding sequences or their recombinant derivatives introduced in tandem with appropriate regulatory sequences (e.g., promoters, untranslated regions, transcription factor binding sites). Yeast pheromones may belong to the family of lipidated pheromones of the a-factor type that are expressed as precursors and secreted using nontraditional pathways or the family of peptide pheromones of the alpha-factor type that are also expressed as precursors but are secreted using the traditional pathway. Several modes of regulation may be used to control and activate biogenesis of both pheromone types. Pheromones of the a-factor family are mostly processed by non-dedicated enzymes. Exceptions are the endoproteases (e.g., AXL1 in S. cerevisiae and C. albicans) and the ABC transporters (e.g., STE6 in S. cerevisiae and HST6 in C. albicans) that are re-sponsible for final maturation steps and secretion of a-factor pheromones. Pheromones of the alpha-factor types are produced and secreted by the standard secretion pathway, hence, their maturation is closely tied to other cellular systems. There is, however, great flexibility in design of open reading frame structure of pheromone precursors. In addition to putative signaling sequences, alpha factor precursors include tandem propeptide repeats that are singled out and further released in a series of processing steps. Hence, biogenesis of alpha factor type pheromones may be modulated through tandem repeat of propeptide sequences and through engineering of the propeptide sequences themselves.

In some embodiments, according to any of the secreted signal objects described herein, the secreted signal object is a small molecule, peptide, or protein capable of being produced and/or secreted by a mammalian cell. In some embodiments, the secreted signal object is a small molecule hormone, a neurotransmitter, a peptide hormone, a neuropeptide, a growth factor, an eicosanoid, a glycoprotein, a chemokine, a monokine, a colony-stimulating factor, a lymphokine, an interleukin, an interferon, or the like.

In some embodiments, biogeneration of signal objects may require heterologous metabolic mechanisms. This may especially be the case if the secreted signal objects are recognized by entities in the collected sample as compared to signal objects that are recognized by other CUs. In such cases, the CU may be metabolically engineered to produce and regulate the various metabolic factors (enzymes, co-enzymes, etc.) that are necessary to produce the signal objects from the native metabolic precursors.

In some embodiments, CUs affect signals indirectly by producing CUs of the same or different type (see FIG. 1F). In cases involving cellular CUs, additional CUs may be produced through either sexual or asexual reproduction. For example, sexual conjugation may be used to fuse individual CUs into a single CU that may exhibit a different phenotype. Additional CUs of possibly the same type may be produced through asexual reproduction (e.g., mitosis or cellular fission). A large number of CUs of different type may be produced through secretion of objects into the medium or onto the cellular surface. In some embodiments, secreted CUs include enzymes that can either affect other secreted CUs or signal objects. Secreted enzymes may include yeast-derived proteases (e.g., BAR1 homologues or derivatives) or heterologously expressed proteases (e.g., bacterial subtilisins or derivatives). In some embodiments, secreted CUs exhibit activity towards system objects contained in the collected sample. Secreted CUs may include lyases (e.g., heparinases, hyaluronidases) with specificity for various distal facing surface layers that serve to modify the target object SBE profile. Secreted CUs may also include cytolytic enzymes (e.g., perforin, granzymes) or their precursors that upon activation serve in target object lysis.

In some embodiments, according to any of the systems described herein comprising a CU capable of producing an SO, the SO is released into a medium comprising the system. In some embodiments, the SO remains localized to a boundary layer around a cluster comprising the CU. In some embodiments, the SO is not localized to a cluster comprising the CU.

In some embodiments, according to any of the systems described herein comprising a CU capable of producing an SO, the produced SO is associated with the CU. In some embodiments, the SO is non-covalently associated with the CU. In some embodiments, the SO is covalently associated with the CU. In some embodiments, the CU is a cell, and the SO is an antigen presented on the surface of the cell.

In some embodiments, according to any of the systems described herein comprising a CU capable of producing an SO, the SO is an internal SO capable of being recognized by a) at least one CU in the system and/or b) at least one sample object in the system. In some embodiments, the SO is capable of being recognized by a CU in the system capable of producing the SO, optionally wherein the CU is capable of recognizing an external SO (e.g., an SO produced by a sample object). In some embodiments, the internal SO and the external SO are the same or functionally equivalent with regard to their ability to modulate an activity of the CU and/or an activity of a sample object capable of producing the external SO. For example, in some embodiments the CU is a cell capable of producing an internal SO, wherein i) the CU comprises a receptor capable of being activated by the internal SO; ii) a cellular sample object in the system comprises a receptor capable of being activated by the internal SO; and/or iii) the cellular sample object is capable of producing an external SO capable of activating the CU receptor of i) and/or the cellular sample object receptor of ii). In some embodiments, the SO is capable of being recognized by another CU in the system. In some embodiments, recognition of the SO by a CU is capable of modulating an activity of the CU. In some embodiments, recognition of the SO by a CU is capable of modulating the ability of the CU to produce an SO. In some embodiments, recognition of the SO by a CU is capable of modulating the ability of the CU to degrade an SO. In some embodiments, the SO is capable of being recognized by a sample object in the system. In some embodiments, recognition of the SO by the sample object is capable of modulating an activity of the sample object. In some embodiments, recognition of the SO by the sample object is capable of modulating the ability of the sample object to produce an SO. In some embodiments, recognition of the SO by the sample object is capable of modulating the ability of the sample object to degrade an SO.

In some embodiments, according to any of the systems described herein comprising a CU capable of producing an SO, the SO is an output SO, and the output signal comprises a level of the output SO. In some embodiments, the SO comprises a fluorescent signal, or is capable of generating a fluorescent signal. In some embodiments, the SO is capable of being detected by an instrument.

In some embodiments, according to any of the systems described herein comprising a CU associated with an SBE and capable of producing an SO, the capability of the CU to produce the SO is enhanced by binding of the SBE to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a CU associated with an SBE and capable of producing an SO, the capability of the CU to produce the SO is attenuated by binding of the SBE associated with the CU to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a CU capable of degrading an SO, the SO is an internal SO capable of being recognized by a) at least one CU in the system and/or b) at least one sample object in the system. In some embodiments, the SO is capable of being recognized by another CU in the system. In some embodiments, recognition of the SO by the other CU is capable of modulating an activity of the other CU. In some embodiments, recognition of the SO by the other CU is capable of modulating the ability of the other CU to produce an SO. In some embodiments, recognition of the SO by the other CU is capable of modulating the ability of the other CU to degrade an SO. In some embodiments, the SO is capable of being recognized by a sample object in the system. In some embodiments, recognition of the SO by the sample object is capable of modulating an activity of the sample object. In some embodiments, recognition of the SO by the sample object is capable of modulating the ability of the sample object to produce an SO. In some embodiments, recognition of the SO by the sample object is capable of modulating the ability of the sample object to degrade an SO.

In some embodiments, according to any of the systems described herein comprising a CU capable of degrading an SO, the SO is an output SO, and the output signal comprises a level of the output SO. In some embodiments, the SO comprises a fluorescent signal, or is capable of generating a fluorescent signal. In some embodiments, the SO is capable of being detected by an instrument.

In some embodiments, according to any of the systems described herein comprising a CU associated with an SBE and capable of degrading an SO, the capability of the CU to degrade the SO is enhanced by binding of the SBE to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a CU associated with an SBE and capable of degrading an SO, the capability of the CU to degrade the SO is attenuated by binding of the SBE associated with the CU to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a sample object capable of producing an SO, the SO is released into a medium comprising the system.

In some embodiments, according to any of the systems described herein comprising a sample object capable of producing an SO, the SO is associated with the sample object.

In some embodiments, according to any of the systems described herein comprising a sample object capable of producing an SO, the SO is an internal SO capable of being recognized by a) at least one CU in the system and/or b) at least one sample object in the system.

In some embodiments, according to any of the systems described herein comprising a sample object capable of producing an SO, the SO is an output SO, and the output signal comprises a level of the output SO.

In some embodiments, according to any of the systems described herein comprising a sample object associated with an SBE and capable of producing an SO, the capability of the sample object to produce the SO is enhanced by binding of the SBE to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a sample object associated with an SBE and capable of producing an SO, the capability of the sample object to produce the SO is attenuated by binding of the SBE associated with the sample object to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a sample object capable of degrading an SO, the SO is an internal SO capable of being recognized by a) at least one CU in the system and/or b) at least one sample object in the system.

In some embodiments, according to any of the systems described herein comprising a sample object capable of degrading an SO, the SO is an output SO, and the output signal comprises a level of the output SO.

In some embodiments, according to any of the systems described herein comprising a sample object associated with an SBE and capable of degrading an SO, the capability of the sample object to degrade the SO is enhanced by binding of the SBE to its cognate binding partner. In some embodiments, the cognate binding partner is a) an SBE associated with at least one CU in the system; b) an SBE associated with at least one sample object in the system; or c) another SO.

In some embodiments, according to any of the systems described herein comprising a sample object associated with an SBE and capable of degrading an SO, the capability of the sample object to degrade the SO is attenuated by binding of the SBE associated with the sample object to its cognate binding partner. In some embodiments, the cognate binding partner is a) an SBE associated with at least one CU in the system; b) an SBE associated with at least one sample object in the system; or c) another SO.

In some embodiments, according to any of the systems described herein, the system comprises a sample object capable of recognizing an SO. In some embodiments, the SO is produced by at least one CU in the system. In some embodiments, recognition of the SO by the sample object is capable of inducing the sample object to produce another SO. In some embodiments, the other SO is an internal SO capable of being recognized by a) at least one CU in the system and/or b) at least one sample object in the system. In some embodiments, the other SO is an output SO, and the output signal comprises a level of the other SO.

In some embodiments, according to any of the systems described herein comprising a sample object associated with an SBE, the SBE is capable of being recognized by a first SBE associated with a first CU in the system. In some embodiments, the SBE associated with the sample object is capable of being directly recognized by the first SBE (see, for example, FIG. 2A). In some embodiments, the SBE associated with the sample object is capable of being indirectly recognized by the first SBE through a second CU in the system. For example, in some embodiments, the SBE associated with the sample object is capable of being recognized by a second SBE associated with the second CU, and a third SBE associated with the second CU is capable of being recognized by the first SBE (see, for example, FIG. 2B). In some embodiments, the second CU is a BsAb comprising a first binding moiety specific for the SBE associated with the given sample object and a second binding moiety specific for the first SBE.

### Signal Recognition

In some embodiments, a CU is affected by internal or external signal objects (see FIG. 1C) through interaction with an SBE. An SBE may be specific for a single signal or multi-specific for a subset of signals with variable sensitivity for each member of the subset. The SBE recognizes a signal through physical interaction, e.g., by hydrogen bonds, van der Waals forces, hydrophobic interactions, ionic interactions. Once an interaction is initiated, the SBE displays some change in activity. In cases where the CU is a molecular entity with an SBE moiety and some assigned enzymatic function, signal interaction may either activate or inhibit the enzymatic process. In some embodiments, the CU may be a protease with an SBE located near the active site where signal interactions lead to protease inhibition. Signal interaction with an SBE does not necessarily prevent processing of the signal object by either the interacting CU or some other CU. For example, further processing of the interacting signal by another protease (e.g., cleavage of either terminal end) may reverse the inhibitory effects of the original signal. Such interactions are well documented for commercially available proteases and protease inhibitors. Their application in biocomputing, however, is unexplored.

In some embodiments, an SBE is located on the surface of a cellular CU with a signaling moiety located on the cytoplasmic side of the cellular membrane. An SBE may, for instance, be a surface receptor or a transmembrane receptor protein. In cases where the CU is derived from a yeast cell, an SBE may be a yeast receptor or its derivative (e.g., the signaling moiety of a yeast receptor fused to an engineered segment). Alternatively, an SBE may include a heterologous signaling moiety fused to a yeast signaling moiety and may incorporate other modifications for improved activity. In such a way, the SBE may incorporate homologous and heterologous segments of G protein-coupled receptors (GPCRs) in yeast derived CUs. Incorporated GPCRs may include modifications (e.g., compositions of extracellular, transmembrane, and cytoplasmic domains of GPCRs from different organisms and mutations improving their signaling properties) that alter the receptors' specificities, sen-sitivities, and signaling activities. Incorporated GPCRs may also include modifications (e.g., mutations or truncations in cytoplasmic domains) that alter posttranslational regulation of receptor activity (e.g., degradation, molecular interaction). Particular yeast GPCRs include, but are not limited to, the S. cerevisiae pheromone receptors STE2 and STE3 or their derivatives (e.g., mutants with altered stability).

In some embodiments, an SBE may be a cytoplasmic protein with a binding moiety (e.g., a DNA binding site). An SBE, for instance, may be a transcription factor with a sensory domain that recognizes membrane permeable signal objects. In some cases, this may include chimeric transcription factors that comprise a regulatory domain fused to a DNA binding domain and a sensory domain. The SBE may exhibit altered binding affinity upon signal recognition and may either repress or activate transcription in its DNA bound state. An SBE may be a transcriptional repressor of the LuxR type that recognizes bacterial autoinducers in natural quorum sensing mechanisms. An SBE may also be any of the commonly used transcription factors (e.g., Lad, TetR) that recognize molecular inducers routinely used to modulate gene expression.

In some embodiments, according to any of the systems described herein, the system comprises a CU configured to respond to an SO in a medium comprising the system. In some embodiments, the CU is configured to respond to an SO in the medium by enhancing the level of the SO in the medium (see, for example, FIG. 15). In some embodiments, the SO is an internal SO capable of being recognized by a) at least one CU in the system and/or b) at least one sample object in the system. In some embodiments, the SO is an output SO, and the output signal comprises a level of the SO. In some embodiments, the CU is capable of producing the SO, and interaction of the SO with the CU enhances the capability of the CU to produce the SO. In some embodiments, the CU is configured to respond to the SO by producing another SO, wherein interaction of the other SO with another CU capable of producing the SO enhances the capability of the other CU to produce the SO (see, for example, FIG. 15).

### Internal States

In some embodiments, a CU stores information regarding past interactions with system objects and external influences in its internal state. For the purposes of the present invention, the internal state of the CU is not necessarily identical to the full state of the physical object as is defined by dynamical systems theory. Instead, the internal state contains information necessary to support and execute future computing actions. The internal state may include continuous variables (e.g., ionic concentrations, permittivities, permeabilities, internal pressures, absorbances, rigidities), discrete variables (e.g., entity copy numbers, degrees of polymerization, set of molecular conformations, molecular modifications), as well as spatial distributions (e.g., compartmentalisation of entities, polarization). In some cases, for the purposes of computing system modeling, it is convenient to define the state through probability distributions.

Internal states may be defined for both molecular and cellular CUs. Internal states of cellular CUs, may be aptly described by biochemical reaction network models. Physical manifestations of the states, as related to the present invention, may be copy numbers of certain molecular species, in particular regulatory molecular species (e.g., transcription factors, regulatory RNAs, transferases) that contribute to maintaining cellular homeostasis. In some embodiments, the physical manifestations of the state include copy numbers of active or inactive transcription factors in select cellular compartments. Wildtype transcription factors or their regulators may be considered (e.g., STE12, WHI5, GAL4, TEC1, DIG1, DIG2). Heterologous transcription factors (e.g., Lad, TetR, LuxR) modified for the given host organism (e.g. fused to a putative nuclear localization sequence) may also be considered. In addition, novel transcription factors (e.g., regulatory domains fused to DNA binding domains) may be considered. The internal state of the molecular CU may be set at time of production or through later interaction with objects or CUs.

### State Storage and Processing

In some embodiments, a CU implements mechanisms that change the state following signal recognition or external influence. The effect of a mechanism may be predicted precisely (e.g., rapid and stable conformational change) or have a stochastic nature (e.g., a change in an entity's time averaged copy number). The state change may happen immediately following interaction of the SBE with its cognate ligand or following external influence. For instance, this may be the case when the SO interacts directly with a transcription factor or if the transcription factor undergoes conformational changes as a result of a shift in temperature, illumination, etc. In some embodiments, the state change follows a transient internal process during which the state change permeates but the effector process is reset upon signal removal. The effector process may include one or more intermediate steps wherein molecular species undergo modifications that build in parallel or in sequence. The effectors may form a cascade, where the first effector modifies the second, and so on. The cascade may also involve additional elements that support or inhibit its progress. Eukaryotic cells implement widely conserved MAPK cascades that make possible various signal processing functions and accept multiple regulators by which the cascades may be redirected and repurposed. The effector process may include an MAPK cascade coupled by adapter proteins and G proteins to signal sensing GPCRs.

The yeast pheromone pathway may be coopted in this way to relay SO recognition to nuclear entities. Various pathway regulators may be altered to modulate the transient response of a wildtype signaling pathway. To improve signal transduction from a GPCR to its G protein, GTPase activity of the G protein may be reduced by modulation (e.g., knockout or downregulation of coding genes or modification of active sites) of GTPase activating proteins (e.g., SST2 in S. cerevisiae). In particular, SST2 may be constitutively expressed using non-native promoters to tune the EC50 value (half-maximal effective concentration) of the pheromone pathway or its reengineered variants. Terminal kinases of MAPK cascades (e.g., FUS3 in S. cerevisiae) activate many downstream processes and may be used to effect state changes. The terminal kinase typically includes a single activating kinase and multiple deactivating phosphatases. In the yeast pheromone response pathway, FUS3 (the terminal kinase) is dephosphorylated by at least three different phosphatases (PTP2, PTP3, and MSG5). The present invention provides methods for increasing the pathway response time, increasing the maximal pathway activity, and lengthening the pathway recovery time by complementary modulation of phosphatase expression. In some embodiments, this includes deletion of certain coding sequences and replacement of regulatory sequences at different loci.

Transient modification of transcription factor complexes following signal recognition may also serve in changing the internal state of a cellular CU. A transcription factor may either be modified directly by the SO (e.g., by an inducer molecule such as IPTG or an autoinducer molecule) or as part of the transient process that ensues following a signal recognition event. Activated transcription factors or associated elements may yield changes in transcription that subsequently alter the many other processes. The yeast pheromone pathway is most often associated with transcription factors that regulate the mating response. Mating response promoters may therefore be used to translate signal recognition events into changes in transcription. In some cases, this may be disadvantageous as the resulting change in internal state may be more extensive than is desired. Alternatively, the pathway may be rerouted to mating response independent promoters using chimeric transcription factors. The native response of the pathway may than be turned off by deletion of the wild-type transcription factor genes. For instance, the S. cerevisiae transcription factor STE12, which is activated following FUS3-mediated dissociation from the inhibitors DIG1 and DIG2, may be used to activate promoters containing putative pheromone response elements. Alternatively, STE12 segments may be fused to wildtype or engineered binding domains (e.g., the DNA binding domain of GAL4) to reroute the pathway to select promoters (e.g., GAL1). Novel transcription factors may also be used to transform signal recognition events into transcriptional changes. As with chimeric transcription factors, novel transcription factors allow pathway rerouting to promoters that are independent of the native response. In addition, novel transcription factors may enable further modulation or signal processing. In one aspect, the present invention provides transcription factors that are dually repressed by two independent events. The family of provided transcription factors comprise three tandem domains coded in a single open reading frame. The N-terminus comprises a pheromone induced nuclear export sequence. The C-terminus comprises a transcription factor that is amenable to N-terminus tagging. The middle flanked segment is a buffer that attenuates passive nuclear transport. The resulting fusion protein is exported from the nucleus upon phosphorylation by active FUS3, responds to inducers of the C-terminus transcription factor, and is recognized by the C-terminus transcription factor promoters. In some embodiments, the resulting transcription factor may therefore be used as a two input logic gate. For instance, when the C-terminal domain is a de-repressible repressor (e.g, Lad), the presence of either pheromone or the inducer will result in active transcription of the associated coding sequence.

In some embodiments, a CU implements mechanisms that process and change the state without external influence. The mechanisms may stabilize the current state by nullifying the effects of random and external actions (e.g., regulation by negative feedback) or execute conditional state transitions that persist (e.g., periodic changes) or terminate in a finite number of steps (e.g., evaluation of logical operations). In cases where the internal state corresponds to a transcriptional network state, a wide range of transcriptional motifs and gene regulation mechanisms are disclosed. Gene regulatory motifs for autoregulation, switching, temporal control, delay, etc., are described in the literature in numerous embodiments.

At any time, the internal state of the CU may affect intracellular entities that are not themselves part of the state. In cases where the state determines gene activities, the current state of the system may determine the copy numbers of the corresponding gene products and thereby the states of any entity those products affect. The affected entities may include SBEs. Hence, the SBE profile of a CU may change as a result of a state change. The affected entities may include SBEs and any elements that relay signal recognition events to other parts of the cell. The affected entities may include produced CUs or signal objects. Note, any of the entities affected through state change may be equally affected by signal recognition events or external influences.

In some embodiments, signal recognition events, external influences, or state changes may also lead to production of reporter entities. A reporter entity is any suitable molecular entity that is capable of affecting quantitative or qualitative measurements. Suitable reporter entities include fluorescent proteins (e.g., GFP, RFP, YFP, CFP), luminescent proteins (e.g., luciferase), and enzymes (e.g., beta-lactamase, beta-galactosidase, SEAP), where the origin of these reporters and the coding sequences are fully disclosed in the current state of the art. In all these cases, the produced reporter entities may be considered cytoplasmic for practical purposes. In some cases, the reporter entities may be secreted and either linked to the surface or released into the medium. Secretion of reporter entities may increase their accessibility or increase their reporting function.

In some embodiments, entities are introduced that are not affected by either signal recognition events, external influences, or internal state changes. These entities may be taken from the same family of entities as the reporter entities (e.g., fluorescent proteins, luminescent proteins, enzymes, etc.) and may be used to generate control measurements to which other measurements are compared.

### Signal Objects (SOs)

Whereas CUs are primarily intended to generate new information regarding system entities, signal objects (SOs) do not generate information and are instead intended to complement CUs by transferring information between entities. For practical purposes, a signal object is any object that interacts with an SBE. In some cases, an object may be both a signal objects and a CU. For instance, a signal object may be fused to a molecular entity that itself has signal processing functions. This may result in signal objects that interact with other signal objects or CUs that are recognized by SBEs. In other cases, signal objects are suspended in the medium before association with their respective SBEs.

Signal objects are either added to a medium comprising the system or are generated by system entities (e.g., CUs and/or sample objects). Signal objects may either be generated in the medium from other signal objects or generated within CUs from elementary metabolic precursors (e.g., by protein expression) and secreted. Suspended signal objects may have various properties that affect their recognition. Some signal objects may be membrane permeable and hence free to interact with cytoplasmic SBEs. Some signal objects may be membrane impermeable and hence may require SBEs that are exposed to the medium (e.g., distally positioned membrane receptors or suspended receptors). In some cases, recognition of signal objects may be inhibited by other mechanisms (e.g., cell wall size exclusion, hydrophobic sequestration) and may require other special treatments.

In some embodiments, signal objects include simple metabolites (amino acids, carbohydrates, ions, etc.), antibiotics (tetracycline, doxycycline, ampicillin, etc.), and various synthetic compounds (Isopropyl beta-D-1-thiogalactopyranoside—IPTG, nitrocefin, anhydrotetracycline—aTc, toxins, etc.). Signal objects, may also include biogenerated entities, e.g., signaling molecules of viral, bacterial, or mammalian origin. Well known families of biogenerated signaling molecules include bacterial acetylated homoserine lactones (AHL) and type 2 autoinducers, yeast mating pheromones, plant hormones, animal morphogens, and a wide array of mammalian hormones, cytokines, interleukins, chemokines etc. Biogenerated signaling molecules may also be engineered for altered specificity or function.

While, through appropriate analysis, most signals may be either qualitatively or quantitatively measured, some signals are better suited for measurement by external devices. For instance, some signals may alter the fluorescence, absorbance, luminescence, electrical impedance, etc. of a medium comprising one or more systems as described herein. Such signals may be used as readouts of the computing systems.

### External Signals

Signal objects that are not produced by CUs are referred to as external signals. External signals of chemical nature may include synthesized molecules (metabolites and related compounds, short peptides, etc.) or purified biogenerated products. Added signal objects may include transcriptional inducers (e.g., IPTG, aTc, AHL), classes of amino acids (e.g., aromatic amino acids), pheromones (e.g., alpha factor, a factor), etc. External signals of non-chemical nature may include optical signals that illuminate a medium comprising the system, magnetic signals that that magnetize system objects, electrical signals that polarize object charge, etc.

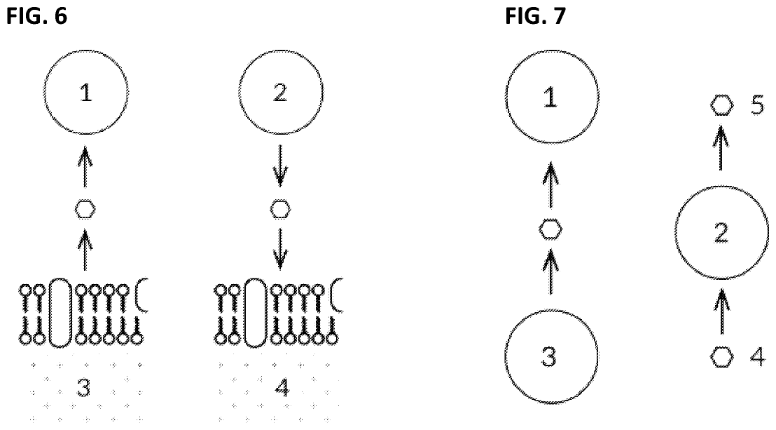

External signals may also be produced by entities in the collected sample (see FIG. 6). Such signal may include a wide array of molecules that are either well-mixed throughout a medium comprising the system (e.g., through a priory accumulation and mixing) or are localized to specific target objects (e.g., by a priory washing of sample objects or active degradation). While well-mixed signals may serve to coordinate system wide computing functions, localized signals will affect only those CUs that recognize the target object, i.e., CUs with SBEs that interact with SBEs displayed by the target object class. Production of external signals may be constant in time or time-varying according to some predetermined pattern (e.g., exponentially decaying in time).

### Internal Signals

Signal objects that are produced by CUs are referred to as internal signals. The internal signal classification does not bar a signal from also being classified as external. For instance, signals that are produced within a medium, isolated, and manually returned to the medium at a later time are both internal and external signals.

Internal signals may be produced from other signals, e.g., by cleavage of existing signals or from metabolic precursors within cellular CUs (see FIG. 7). In some embodiments, internal signals may be engineered for altered behavior. Signal objects, or enzymes contributing to their production, may be expressed from recombined genes that comprise specific regulatory sequences (e.g., promoters, operators, untranslated regions). Signal behavior may be engineered through addition of synthetic open reading frame (ORF) elements. In some cases, it is possible to modulate signal degradation by extending the terminal domains with a peptidase recognition tag. Peptide signals may also be translated as prepro-peptides, where a series of additional cytoplasmic, periplasmic, or extracellular processing steps are required to produce mature pheromones.

The prepro-peptide format provides a platform for engineering signal activation, strength, and specificity and may therefore increase informational content of a single internal signal object. A signal object may have multiple activity states each characterized by its affinity for SBEs. Cleavage of propeptide sequences may transition signal objects between the activity states and putative protease recognition sites within the propeptide sequence may be used to encode such transitions.

In some embodiments, the signaling peptide belongs to or is derived from the family of lipidated or non-lipidated yeast pheromones (e.g., the S. cerevisiae alpha or a mating factors). Yeast pheromone propeptide sequences may be engineered to increase or decrease the signal strength of a single pre-propeptide by varying the number of mature peptides encoded within a single ORF, e.g., pheromone coding sequences may be flanked by protease recognition sites and repeated within a single ORF. The yeast pheromone alpha factor is translated as a prepro-peptide with up to four mature peptide coding sequences. In engineered configurations, the number of coding sequences may be increased or decreased using the same flanking motifs established in the wildtype sequence. The propeptide sequence may also be extended to increase the number of activity states. Additional sequences that include recognition sites for non-native proteases may be inserted into wildtype sequence. By such modification intermediate activity states may be produced wherein the signal object recognizes SBEs that are different from those recognized by the fully mature pheromone.

The mature pheromone coding sequence may also be altered. As previously disclosed, single codon changes may redirect the signal to non-native SBEs. Alternatively, mature pheromone sequences from one species may be exchanged for pheromone sequences from another species. Previous work has shown that crosstalk between pheromone-receptor pairs from different species may be negligible.

Internal signals may also include signaling molecules from unrelated species. Such internal signals may exhibit properties desirable for some applications. Certain signals (e.g., bacterial autoinducers) may be membrane permeable and hence detectable by simple mechanisms. Other signals (e.g., plant hormones) may provide complete orthogonality with respect to other signal objects in a medium comprising the system. In some embodiments, signal objects may be targeted towards SBEs of target objects within the collected sample (see FIG. 6). Such signals may include mammalian cytokines (e.g., interleukin 2), chemokines, and, more specifically, interleukins, growth hormones, neuropeptides. In each case, recombination technologies may be used to produce heterologous signals in various cellular CUs. Yeast cells provide a cellular platform wherein signals of various origins (e.g., bacterial, mammalian, or other higher eukaryotes) may be produced. In certain embodiments, production of such signals requires additional metabolic engineering to enable necessary posttranslational modifications and metabolic processing.

Internal signals may also exhibit properties that enable their easy measurement by external devices. Measurable signals may include molecules with high specificity that may be quantified directly, e.g., by immunosorbent assay, polymerase chain reaction. Measurable signals may also include molecules that alter bulk properties of a medium comprising the system, e.g., absorbance, fluorescence, etc. Internal signal that are easily measurable are primarily used for readout of device states and are discussed more in the sequel.

### Configurations

An embodiment of the computing system comprising the described entities, e.g., computing units and signals, is programmed through its composition. Functions, made up in part from arithmetic operators, comparison operators, logical operators, etc., are encoded and evaluated by systems of computing units and external signals that interact and self-organize within a medium comprising the system. The input that drives the computation is the collected sample. The composed computational system is able to efficiently solve computational problems, e.g., tree structure searches, that are known to be challenging for current technologies.

From the perspective of computing theory, the invented computing device is able to implement a rich set of operations that includes various combinational logic and sequential logic. In other words, the fundamental configurations provided below are the building blocks that together form a functionally complete system of boolean operations. Moreover, the internal states of the computing units (see section on Computing Units) jointly make up the composite system state that can condition future boolean operations for sequential logic functions.

### Computing Units

The CUs provided by the invention enable a rich set of behaviors. Standard mathematical notation is introduced to describe certain orchestrated functionalities of CU compositions. The following canonical form, however, is not an exhaustive depiction of all embodiments that are intended.

In the canonical form, the following sets describe the objects, SBEs, and signals present in the system. The set of object types is given by Q={1, 2, . . . }. The set of SBE types is given by B={1, 2, . . . }. The set of internal and external signal types is given by Z={1, 2, . . . }. In the canonical form, the set of signals may describe both the signal objects as well as the set of values (e.g., concentrations, copy numbers) that can be assigned to the object types. For example high concentration of a specific pheromone may be assigned the index 1 and a low concentration of the same pheromone may be assigned the index 2.

A particular object (CU or an object from the collected sample) is then described by the signals and SBEs it recognizes, the signals it produces, and by its input-to-output behavior that determines how these properties change following signal recognition events. For each q∈Q, this behavior is compactly given by the following tuple G_{q}=(X_{q}, x_{0,q}, R_{q}, U_{q}, Y_{q}, D_{q}, H_{q}, g_{q}). The individual components of the tuple are clarified next.

The set of discrete states is given by X_{q}={1, . . . , n_{q}}. State restriction to finite states is an abstraction intended to describe progression through either the internal steady states or through the discretized state space. The initial state of the unit is given by x_{0,q}. This is typically the state assumed after the unit is produced or the state achieved following unit pretreatment.

The set of signals a unit can recognize is a function of the current state x_{q} ∈X_{q} and is given by the set R_{q} (x_{q})⊆Z. Similarly, the set of signals the unit can degrade is given by the set U_{q} (x_{q})⊆Z, the set of signals the unit is producing is given by the set Y_{q} (x_{q})⊆Z, the set of SBEs the unit is displaying is given by the set D_{q} (X_{q})⊆B, and the set of SBEs the unit can recognize is given by the set H_{q} (x_{q})⊆B. For simplicity, the canonical form does not track the number of SBE copies, the sensitivity of signal recognition, or the strength of signal production/degradation.

The function g_{q} describes the dependence of CU state transitions on the current state and on the recognized signals. Signal recognition is represented by Boolean valued functions r_{q,i}, where r_{q,i} evaluates as TRUE if the corresponding signal i∈R_{q} (X_{q}) is currently recognized by the SBE of the object q. By definition, only signal recognition events r_{q,i}, where i∈R_{q} (X_{q}), may evaluate as true.

Affirmative signal recognition depends on many factors including the states and types of signal producing and signal recognizing objects. Herein, a single distinction is used, global signal recognition versus local signal recognition. Local signal recognition is defined solely on the basis of co-localization of signal producing, signal degrading, and signal recognizing objects. Global signal recognition will inherently depend on many factors, e.g., relative strength of the signal with respect to the SBE. Hence, in order to capture global signal recognition in some meaningful way, the dependence of signal production and signal recognition on the object type and state must be described. For the sake of simplicity, this description is omitted and global signaling is omitted from the standard notation (embodiments regarding global signaling are treated in the sequel).

Co-localization of objects can be uniquely determined from the canonical form of those objects. Two objects co-localize if they associate with one another or if they jointly associate with another object or an object cluster. Specifically, two objects are known to co-localize if any of the following conditions hold: 1) the objects recognize each other's SBEs, 2) the objects recognize a mutual SBE, or 3) the objects recognize different SBEs that manifest on a single object or a linked cluster of objects.

This is expressed using the canonical form as follows. Define the set of clusters C that includes all maximal subsets c⊆Q of interconnected object types. In other words, c∈C if for any k₁, kₙ∈c, there exists a sequence (k₂, k₃, . . . , kₙ₋₁), and a related sequence (l₁, l₂, . . . , lₙ₋₁), that satisfy the expression lᵢ∈Dₖ_{ᵢ}(xₖ_{ᵢ})∩Hₖ_{ᵢ₊₁} (xₖ_{ᵢ₊₁}), for all i=1, . . . , n. Note, the clusters may not be disjoint. In other words, some object types may belong to two or more clusters. There are two possible explanations how such a set of clusters is consistent with various embodiments. The first explanation supposes SBE recognition is stochastic, i.e., objects associate and dissociate at random. Hence, clusters may be interpreted as possible instantiations of this random behavior. The second explanation supposes that there are many copies of each object type. As such, it is without contradiction that, even in the case of practically irreversible associations between SBEs, all clusters are realized.

Local signal recognition is described next. Consider a cluster c∈C, where the current state of any object i∈c is given by xᵢ. Then, an object q∈c locally recognizes a signal i∈Z (i.e., r_{q,i} is affirmative), if and only if, (1) i is recognized by q, i.e., i∈R_{q} (x_{q}); (2) i is produced by some CU in the cluster, i.e., there exists q′∈c, such that i∈Y_{q} (x_{q′}); and (3) i is not degraded by an CU in the cluster, i.e., there does not exist q″∈c, such that i∈U_{q″} (x_{q″}).

The composite behavior of the computing device is derived by composition of the individual CU canonical forms in repeated evaluation of localization events (i.e., association and disassociation of objects) and signaling events. Initially, the system is composed and the first set of clusters (i.e., the set C) is computed. Then, for each cluster separately, signaling events within the cluster are identified and the internal states of cluster specific CUs are updated accordingly. An iterative loop ensues, wherein signaling events lead to state changes that lead to more signaling events, and so on. The loop may be interrupted if a state change disrupts a cluster by causing an SBE profile change. Loop interruption is followed by redefinition of canonical forms and re-clustering of the newly formed set of objects Q.

Signaling networks generated within object clusters encode propositional formulas that determine the existence of specific SBE combinations within the associated cluster. CUs of a single type (i.e., CUs having identical canonical forms) provide a single bit of information regarding a cluster's SBEs. This bit of information is limited to disjunctive statements regarding the presence of select SBE types. Signaling enables assembly of information from different CU types. In the absence of physical constraints, the set of propositional statements that are enabled by signaling is functionally complete.

Canonical forms of embodiments describing a minimal functionally complete set of operators are provided next. Signaling networks that realize arbitrary combinational logic are provided by recursion. A cluster is considered separately. First, the canonical form of a trivial target object is described. Next, signaling networks that generate simple propositions regarding individual SBEs are provided. Lastly, various signaling networks that integrate simple propositions to realize basic logical operations are given and cast into a general recursive framework.

### Target Objects

Without loss of generality, consider a trivial target object q₀∈Q. A simple internal structure that is sufficient for the following exposition comprises a singleton state space (i.e., X_{q0}={x_{q0}}) and empty signal recognition, degradation, and production spaces (i.e., R_{q0} (x_{q0})=U_{q0}(x_{q0})=Y_{q0}(x_{q0})=0). The SBE profile of q₀ includes some positive number of members (i.e., D_{q0} (x_{q0})={s₁, s₂, . . . }. The other automata elements, i.e., recognized SBEs given by H_{q0} and internal logic given by g_{q0}, are fully determined by the other entries. Hence, a trivial target object is fully characterized by the element D_{q0}.

### Pheroimmuno Propositions

The most basic function of the computing device is to transpose SBEs into signals. In some implementations, such signals will be considered to be Boolean, and, in part due to the usage of pheromone signals in certain embodiments, the transpositions will be referred to as pheroimmuno propositions (PIPs).

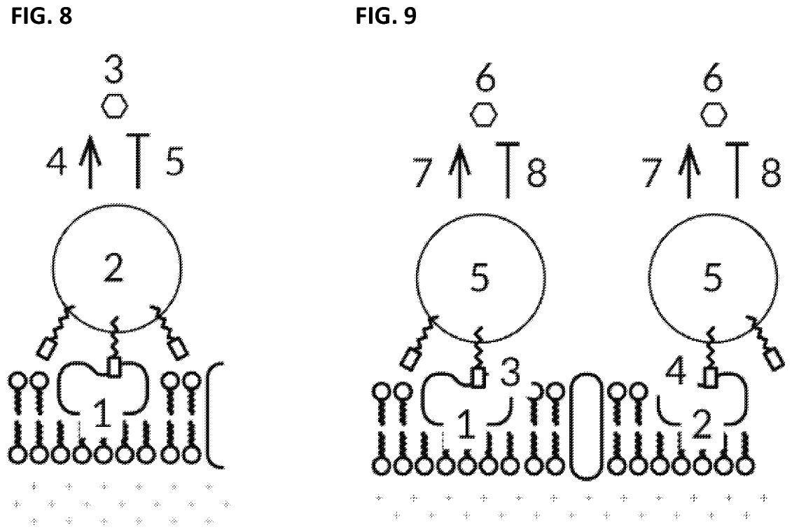

Hence, signals, produced either following a CU's recognition of an SBE or following signaling events triggered as a consequence of SBE recognition, provide Boolean data that characterizes the SBE profile of the target object. The most elementary PIP (see FIG. 8) returns true when an SBE is recognized by a constitutive signal producer. Take a trivial target object q₀ that displays the SBE s₁. Consider a second object q₁∈Q, where, for some x∈X_{q1} and z∈Z, z∈Y_{q1} (x) and s₁∈Y_{q1} (x). Such objects become clustered and the s₁-to-z PIP is becomes affirmative, at least while q₁ is in the state x.

### Logical Operators

In some embodiments, the systems described herein can be used to perform Boolean logical operations. The systems can comprise modules that act as logic circuits that can perform logical operations such as those performed by AND gates, NAND gates, OR gates, NOR gates, XOR gates, XNOR gates, NOT gates, etc. An exemplary logical operation module can receive one or more input signals (e.g., sample objects) and can generate one or more output signals (e.g., output SOs). In some embodiments, a logical operation module as described herein comprises one or more CUs.

### OR Modules

In general, evaluation of compound PIPs requires construction of interconnected signaling networks. However, in the case of elementary disjunctive operations (e.g., logical OR operations on SBEs), the above approach used to construct elementary PIPs is sufficient (see FIG. 9). Consider the two SBEs, s₁ and s₂, and define corresponding boolean variables bₛ_{₁} and bₛ_{₂} that denote their presence. To realize the function s1+s2: =bₛ_{₁}∨bₛ_{₂}, consider an object q₁∈Q, where, for some x∈X_{q}_{₁} and z∈Z, z∈Y_{q}_{₁} (x) and s₁, s₂∈H_{q}_{₁} (x). Suppose a trivial target object q₀∈Q displays only the SBE s₁. In this case, the objects q₀ and q₁ are clustered and the z PIP becomes affirmative, at least while q₁ is in the state x. The same is true if q₀ displays only the SBE s₂ or if q₀ displays both SBEs s₁ and s₂. The z PIP remains negative if and only if q₀ displays neither s₁ nor s₂. Hence, the z PIP computes the Boolean summation s₁+s₂.

In some embodiments, a logical operation module as described herein can operate as an OR gate, wherein it generates an output signal when either a first input signal or a second input signal are present or when both the first input signal and the second input signal are present (e.g., present in the vicinity of the module). In some embodiments, a logical operation module as described herein can operate as a NOR gate, wherein it can generate an output signal when both the first input signal and the second input signal are absent (e.g., absent in the vicinity of the module).

In some embodiments, according to any of the systems described herein, the system comprises a logical OR module comprising a first CU, wherein the first CU comprises a first CU SBE and a second CU SBE, and the first CU SBE is capable of binding to a first cognate binding partner and the second CU SBE is capable of binding to a second cognate binding partner. In some embodiments, the first CU is capable of producing a first SO, binding of the first CU SBE to the first cognate binding partner enhances the capability of the first CU to produce the first SO and binding of the second CU SBE to the second cognate binding partner enhances the capability of the first CU to produce the first SO (see, for example, FIG. 9). In some embodiments, the first CU is capable of degrading a first SO, binding of the first CU SBE to the first cognate binding partner enhances the capability of the first CU to degrade the first SO and binding of the second CU SBE to the second cognate binding partner enhances the capability of the first CU to degrade the first SO (see, for example, FIG. 9).

In some embodiments, according to any of the systems described herein comprising a logical OR module, the first cognate binding partner is a first sample object SBE associated with at least one sample object in the system and the second cognate binding partner is a second sample object SBE associated with at least one sample object in the system.

### AND Modules

Computing other functions necessitates signaling between CUs. Clustering of CUs and target objects alone is insufficient to compute functions comprising conjunctive and negating operations. The invention provides mechanisms for computing conjunctive propositions by conditioning PIPs on other PIPs (see FIG. 10).

Consider two SBEs, s₁ and s₂, and define the corresponding boolean variables bₛ_{₁} and bₛ_{₂} that return true if the given SBE is present on the surface of a target object. To realize the AND function s₁s₂:=bₛ_{₁}∧bₛ_{₂}, introduce two objects q₁, q₂∈Q with state spaces comprising high and low states, i.e., X_{q}_{₁}={x₁} and X_{q}_{₂}={x_{2L}, x_{2H}}. Let the object q₁ recognize the SBE s₁ and constitutively produce the signal z₁ E Z. Let the object q₂ recognize the SBE s₂ and conditionally produce the signal z₂ in the high state x_{2H}, i.e., Y_{q2} (X_{2L})=0 and Y_{q2} (X_{2H})=z₂. Make the signaling unidirectional from q₁ to q₂ and state independent, i.e., R_{q2} (x)={z₁} for all x∈x_{q2}. Finally, condition the state transition of q₂ on signal recognition of z₁, i.e., g_{q2} (X_{2L}, r_{q2,z1}=TRUE)=x_{2H}. The nature of the state transition can vary depending on the internal processing mechanisms (i.e., the transition may be regulated, tracking, or persistent).

Next, consider the dependence of the z₂ PIP on the target object. The z₂ PIP is true only if q₂ is in the cluster and q₂ is in the high state x₂H. Suppose a trivial target object q₀ is missing the SBE s₂. Then the z₂ PIP computes false because q₂ does not cluster with q₀. Now consider a trivial target object that is missing the SBE s₁. Then the z₂ PIP also computes false because q₁ does not cluster with q₀ and hence q₂ remains in the z₂ non-producing state. Finally, consider a trivial target object q₀ displaying both s₁ and s₂. In the absence of other objects, e.g., signal degrading objects, similar analysis shows that in this case the z₂ PIP computes true, i.e., q₀ clusters with q₁ and q₂, q₂ recognizes z₁, and transitions to the z₂ producing state x_{2H}. Hence, this composition computes the Boolean multiplication s₁s₂.

Note some straight forward extensions of this system. By adding additional layers to the signaling cascade, any number of Boolean multiplications can be performed. Consider a set of SBEs s₁, s₂, . . . , sₙ. The function Πᵢ₌₁ⁿsᵢ is computed by the last PIP in a signaling network of n objects where object qᵢ produces a signal that induces signal production by the object qᵢ₊₁. Furthermore, any of the terms in this expression may be replaced with a summation of the form sⱼ₁+sⱼ₂+ . . . . Summation of such expressions may also be computed with parallel cascades enabled by signal combinations that agree only in the terminal signal. Hence, by the methods provided thus far, one may compute any Boolean function of the form

$${Σ}_{i} ( {Π}_{j} ( {Σ}_{k} {s}_{{c}_{i , j , k}} ) ) ,$$

where c_{i,j,k}∈{1, . . . , n}.

In some embodiments, a logical operation module as described herein can operate as an AND gate wherein it generates an output signal only when both of a first input signal and a second input signal are present (e.g., present in the vicinity of the module). In some embodiments, a logical operation module as described herein can operate as a NAND gate, wherein it can suppress/diminish an existing SO/CU when both the first input signal and the second input signal are present (e.g., present in the vicinity of the module).

In some embodiments, according to any of the systems described herein, the system comprises a logical AND module comprising a first CU and a second CU, wherein the first CU comprises a first CU SBE and the second CU comprises a second CU SBE, the first CU SBE is capable of binding to a first cognate binding partner and the second CU SBE is capable of binding to a second cognate binding partner. In some embodiments, the first CU is capable of producing a first SO, the second CU is capable of producing or degrading a second SO, binding of the first CU SBE to the first cognate binding partner enhances the capability of the first CU to produce the first SO, and recognition of the first SO by the second CU enhances the capability of the second CU to produce or degrade the second SO, and clustering of the first and second cognate binding partners in a computational cluster enhances the effect of the first SO such that the first SO produced by the first CU bound to the first cognate binding partner is capable of inducing the second CU bound to the second cognate binding partner to produce or degrade the second SO (see, for example, FIG. 10).

In some embodiments, according to any of the systems described herein comprising a logical AND module, the first cognate binding partner is a first sample object SBE associated with a sample object in the system and the second cognate binding partner is a second sample object SBE associated with the sample object.

### NOT Modules

In some embodiments, a logical operation module as described herein can operate as a NOT gate, wherein it can suppress/diminish an existing SO/CU when a first input signal is present (e.g., present in the vicinity of the module).

### XOR Modules

In some embodiments, a logical operation module as described herein can operate as an XOR gate, wherein it generates an output signal when either a first input signal or a second input signal are present (e.g., present in the vicinity of the module). The logical operation module operating as an XOR gate can suppress an output signal when either both the first and the second input signals are present or when both the first and the second input signals are absent.

### XNOR Modules

In some embodiments, a logical operation module as described herein can operate as an XNOR gate, wherein it generates an output signal when either both the first and the second input signals are present (e.g., present in the vicinity of the module) or when both the first and the second input signals are absent (e.g., absent in the vicinity of the module). The logical operation module operating as a XNOR gate can suppress an output signal when either a first input signal or a second input signal are present (e.g., present in the vicinity of the module).

### NEGATION Modules

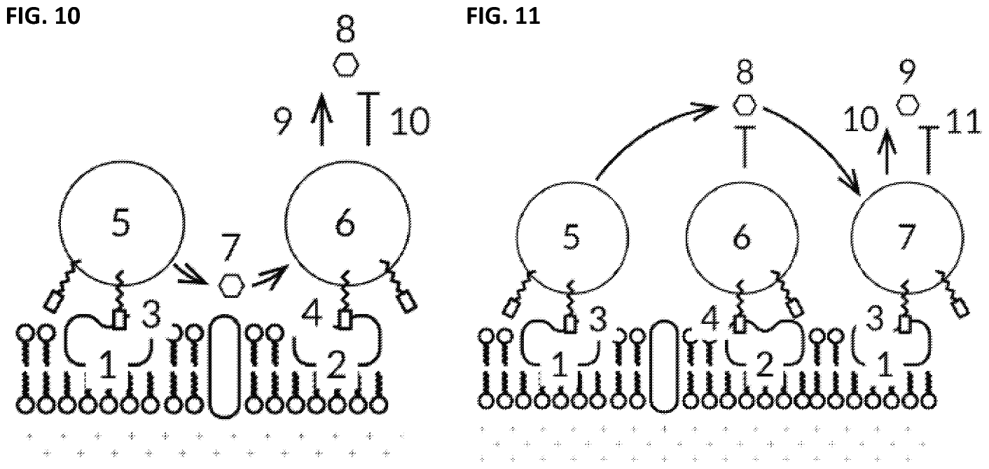

Negation is an essential aspect of any general boolean network as it allows for recognition of absent SBEs. Implication denoted by → is a boolean operation on two inputs wherein the output is false only if the first input is true and the second input is false. Negated implication denoted by [symbol] is a negation of all outputs so that the output is true only if the first input is true and the second input is false. To compute a negated implication, the above configurations are further extended with signal degrading objects (see FIG. 11). In some embodiments, signal degradation is achieved by target associated CUs. This may comprise secretion of signal degrading enzymes or display of signal degrading enzymes by target associated CUs (cellular or molecular).

Consider two SBEs, s₁ and s₂, and define the corresponding boolean variables bₛ_{₁} and bₛ_{₂} that return true if the given SBE is present on the surface of a target object. To realize the negated implication function s₁s̅₂̅:=bₛ_{₁} [symbol] bₛ_{₂}, introduce the two objects q₁, q₂∈Q that implement the function s₁s₁ as described above. Next, introduce a third object q₃ that recognizes the SBE s₂ and constitutively degrades the signal z₁. The object q₃ has no other signaling functions.

Next, consider the dependence of the z₂ PIP on the target object. The z₂ PIP is true only if q₂ is in the cluster and q₂ is in the high state x_{2H}. Suppose a trivial target object q₀∈Q is missing the SBE s₁. Then the z₂ PIP computes false because q₂ does not cluster with q₀. Now consider a trivial target object that is missing s₂. This case reduces to the conjunctive example provided earlier. In the absence of s₂, q₁ and q₂ evaluate s₁s₁. Hence the z₂ PIP evaluates as true if the target object displays s₁. Finally, consider a trivial target object q₀ displaying both s₁ and s₂. In this case, the presence of q₃ leads to degradation of z₁. As a result, q₂ remains in the low state and the z₂ PIP returns false. This exhaustive analysis shows that this composition computes the negated implication s1s2.

Note again the straight forward extension of this system to more general functions. Consider a set of SBEs s₁, s₂, . . . , sₙ. To compute the function Π_{i∈N}sᵢΠ_{i∈M}s̅_̅{̅ι̅}̅ (or summations of such expressions), where N, M⊆{1, . . . , n}, replace the signaling cascade realized by object q₁, q₂ by a signaling cascade that realizes the product Π_{i∈N}s₁. Apply De Morgan's law to rewrite the second product as Π_{i∈M}s̅_̅{̅ι̅}̅=(Σ̅_̅{̅ι̅∈̅M̅}̅s̅_̅{̅ι̅}̅). Modify q₃ so that it recognizes all SBEs in the set M while still degrading the first signal in the cascade. Summations of such expressions may be computed again by replicating the pattern, each time using unique signal combinations where only the terminal signal is retained.

In addition to the generalizations, the provided realization may also be extended to the simple NOT operator. Standalone negation implies a PIP must compute true in the absence of a particular SBE. Hence, a global signal removed from positively evaluated target objects is necessary. The canonical form does not explicitly consider global signaling, however, a constitutive global signal z_{g} ∈Z may be generated by introducing a universal SBE s_{g} that is displayed by all objects and is recognized by a particular CU q_{g} that produces z_{g}. As such, for an SBE s₁, the function s̅₁̅ may be evaluated by a CU q₁∈Q that constitutively recognizes the SBE s₁ and constitutively degrades the signal z_{g}.

In some embodiments, according to any of the systems described herein, the system comprises a logical NEGATED IMPLICATION module comprising a first CU, a second CU, and a third CU, wherein the first CU comprises a first CU SBE, the second CU comprises a second CU SBE, the third CU comprises the first CU SBE, the first CU SBE is capable of binding to a first cognate binding partner, and the second CU SBE is capable of binding to a second cognate binding partner. In some embodiments, the first CU is capable of producing a first SO, the second CU is capable of degrading the first SO, the third CU is capable of producing or degrading a second SO, binding of the first CU SBE of the first CU to the first cognate binding partner enhances the capability of the first CU to produce the first SO, binding of the second CU SBE of the second CU to the second cognate binding partner enhances the capability of the second CU to degrade the first SO, and recognition of the first SO by the third CU enhances the capability of the third CU to produce or degrade the second SO, and wherein clustering of the first cognate binding partners enhances the effect of the first SO such that the first SO produced by the first CU bound to the first cognate binding partner is capable of inducing the third CU bound to the first cognate binding partner to produce or degrade the second SO; and clustering of the first and second cognate binding partners in a computational cluster enhances the effect of the second CU such that the second CU bound to the second cognate binding partner is capable of attenuating induction of third CU by degrading the first SO produced by the first CU bound to the first binding partner (see, for example, FIG. 11).

In some embodiments, according to any of the systems described herein comprising a logical NEGATED IMPLICATION module, the first cognate binding partner is a first sample object SBE associated with a sample object in the system and the second cognate binding partner is a second sample object SBE associated with the sample object.

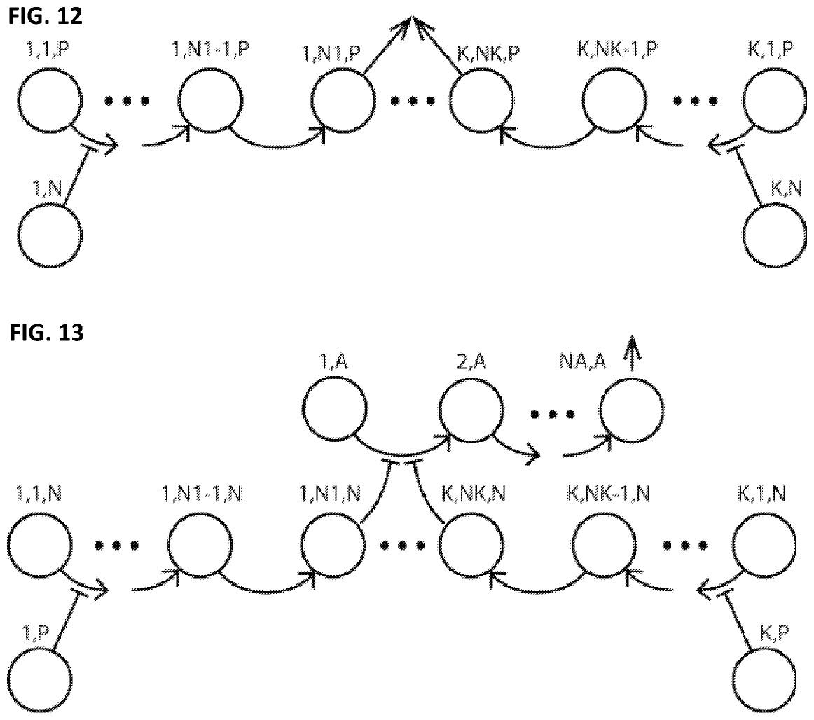

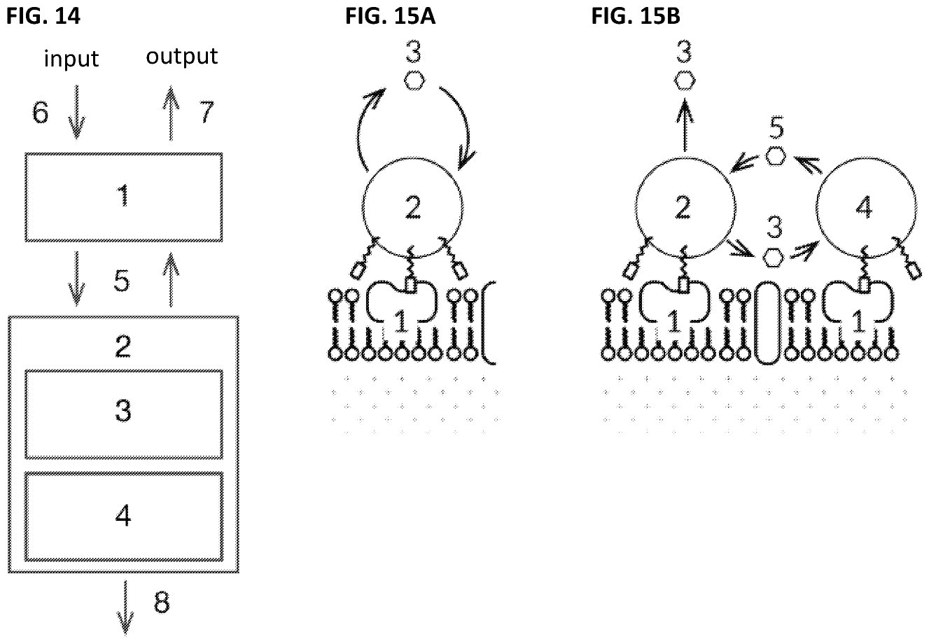

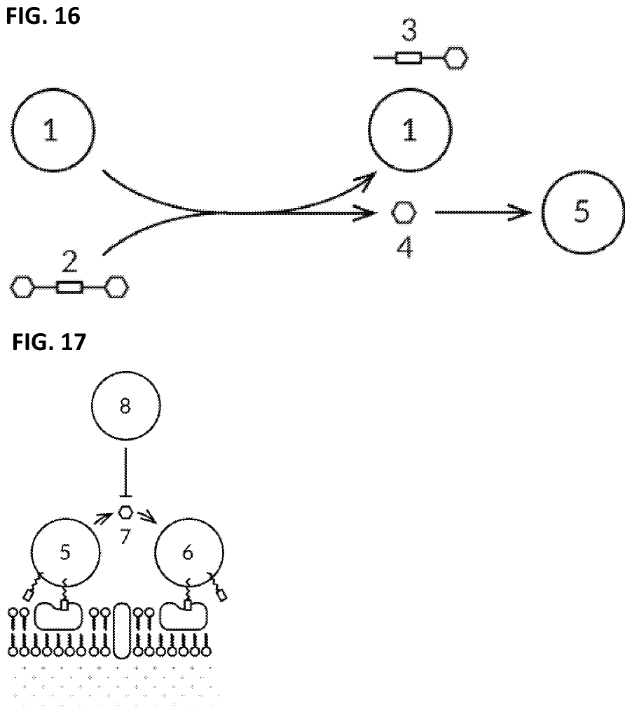

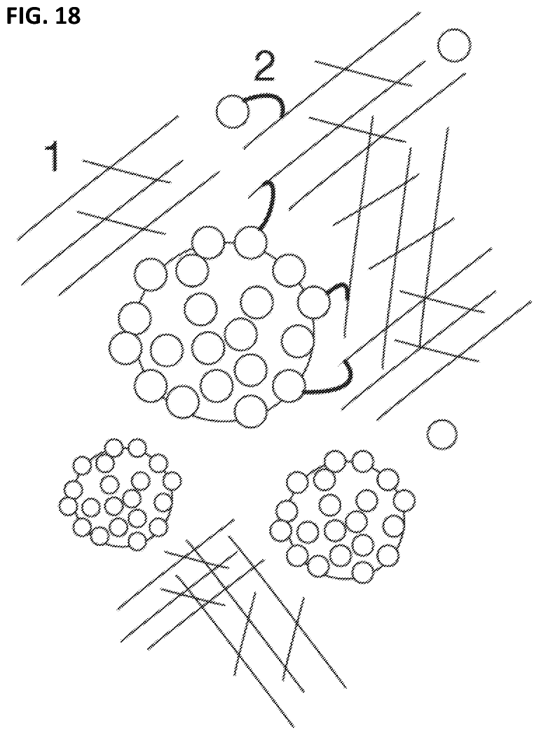

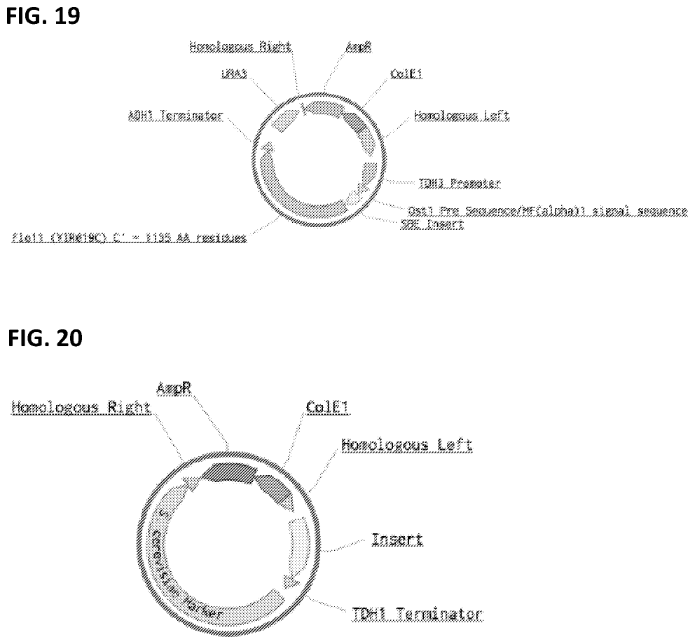

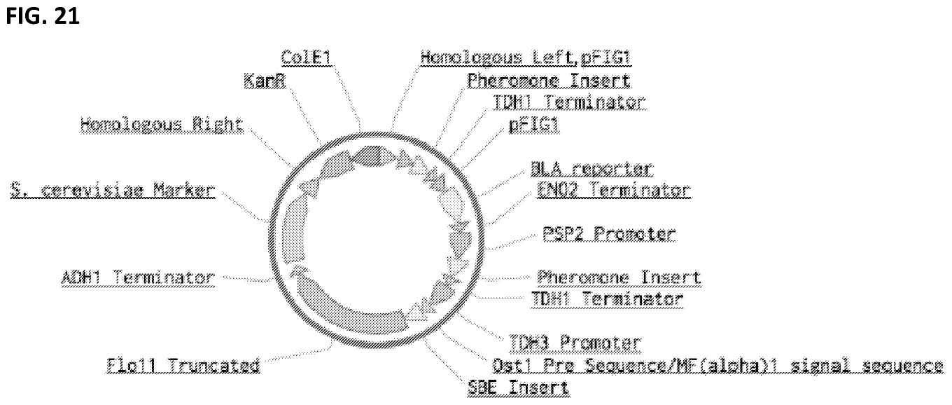

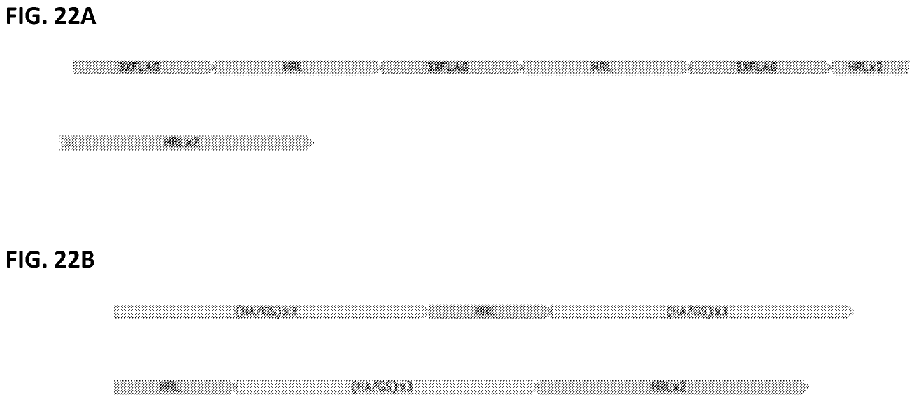

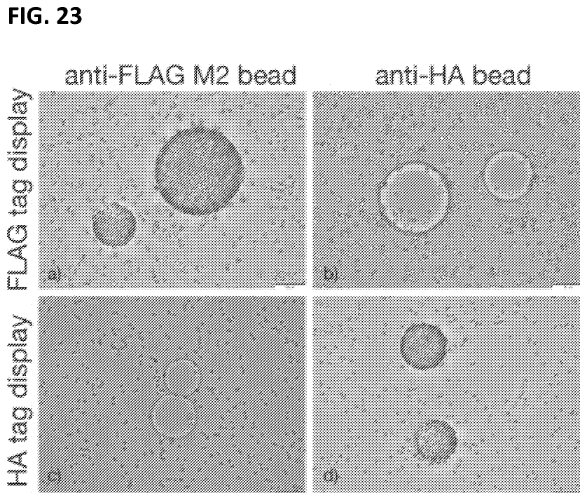

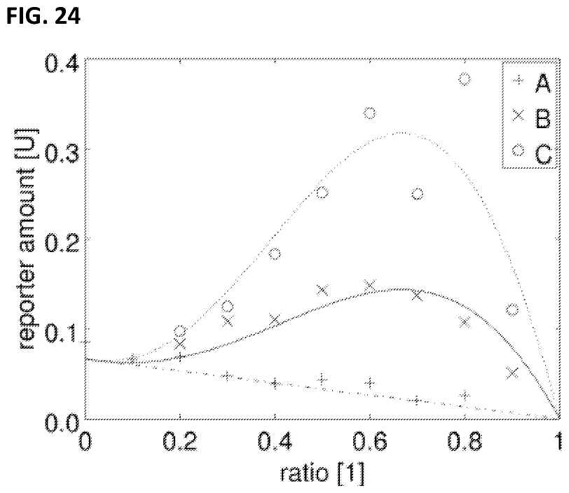

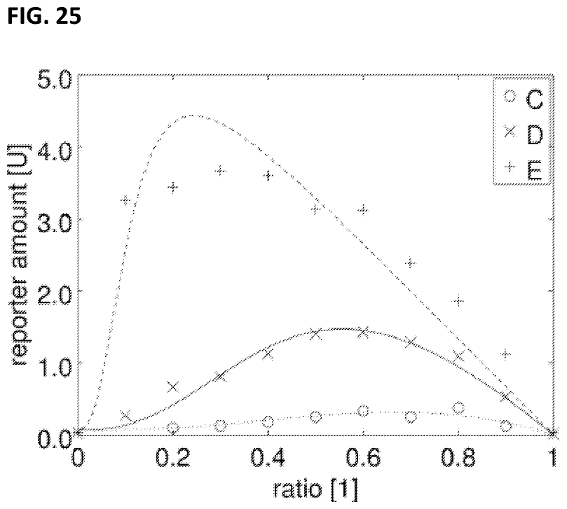

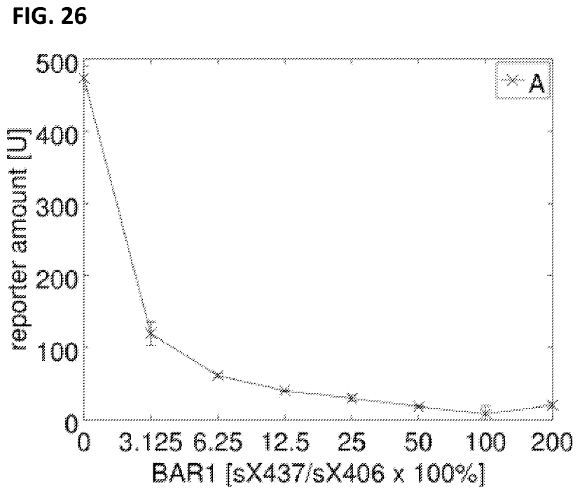

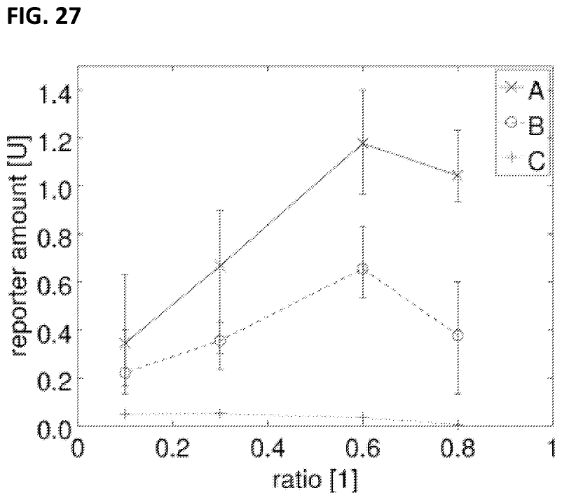

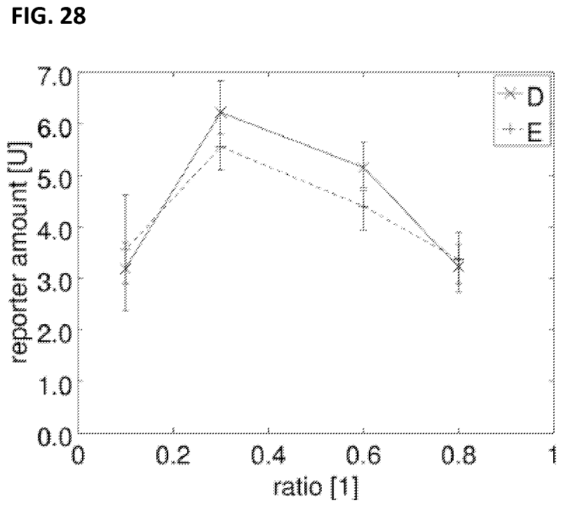

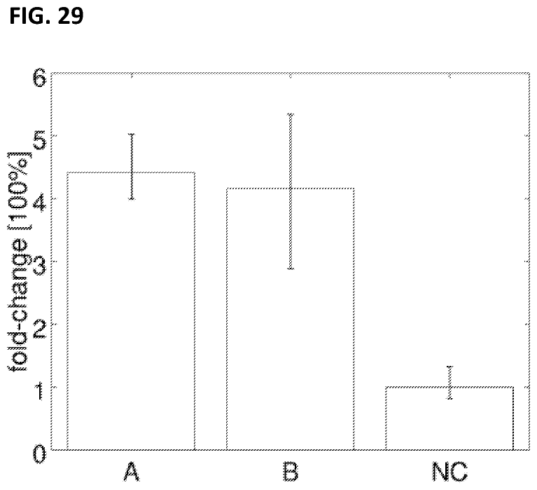

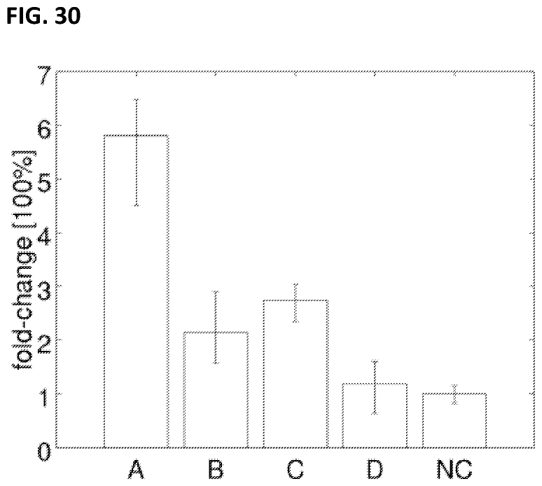

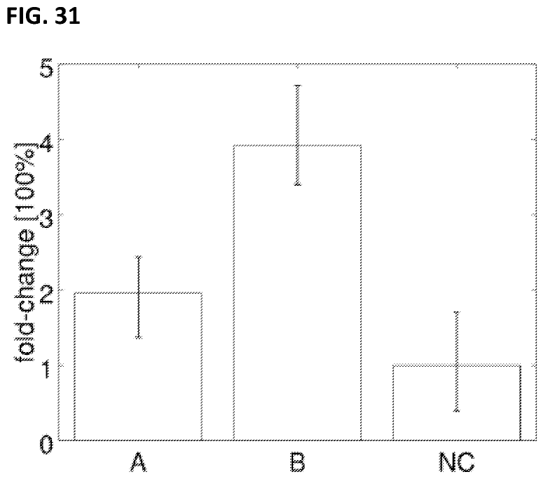

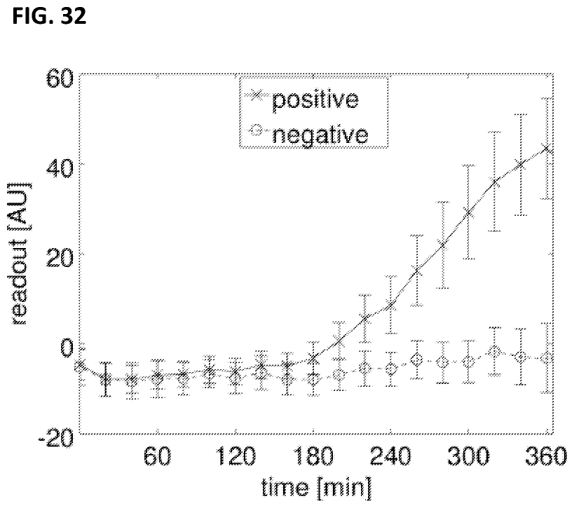

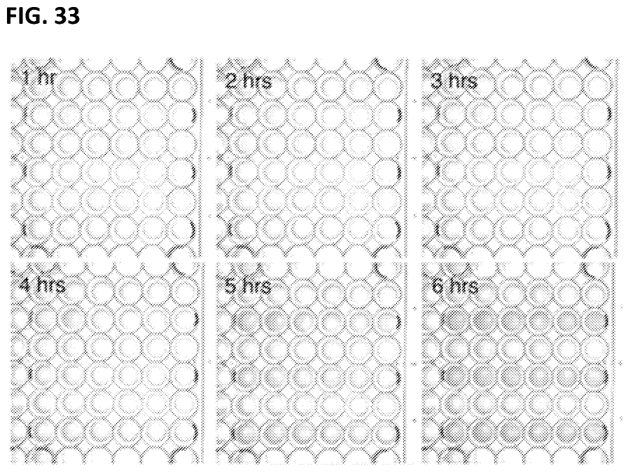

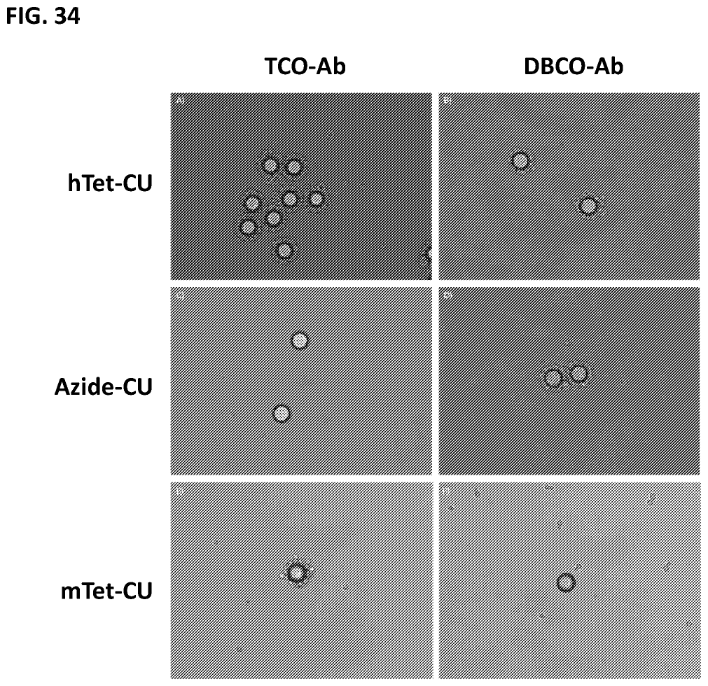

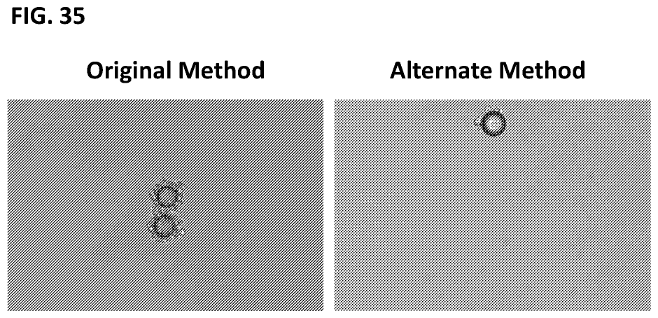

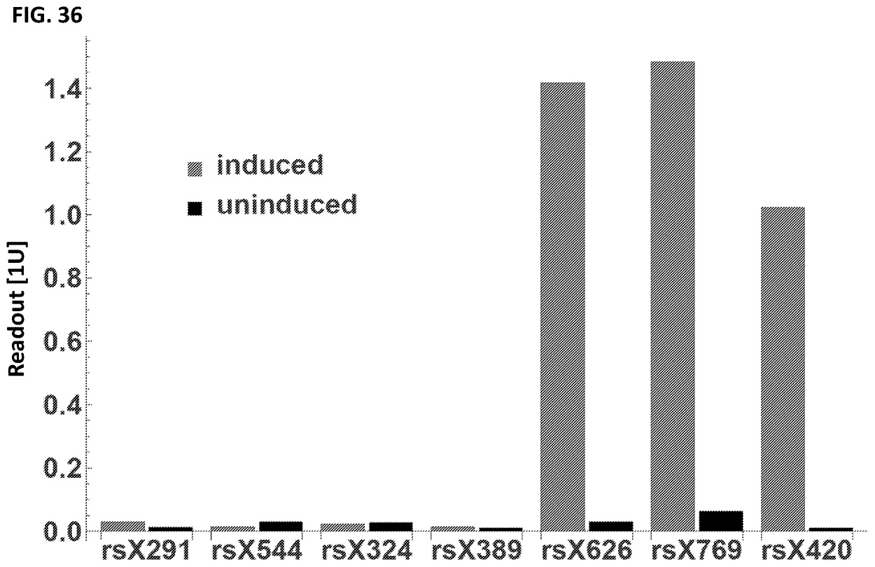

In some embodiments, according to any of the systems described herein, the system comprises one or more logical OR modules, one or more logical AND modules, and/or one or more logical NEGATED IMPLICATION modules (see, for example, FIGS. 12 and 13).

### General Logic Operators

A well-known result from Boolean algebra states that any Boolean function over the variables b₁, . . . , bₙ may be transformed into a standard sum-of-products (SoP) form ΣⱼΠ_{i∈N}_{ⱼ}bᵢΠ_{i∈M}_{ⱼ}b̅_̅{̅ι̅}̅ or product-of-sums (PoS) form Πⱼ(Σ_{i∈N}_{ⱼ}bᵢ+Σ_{i∈M}_{ⱼ}b̅_̅{̅ι̅}̅), where j=1, 2, . . . and Nⱼ, Mⱼ⊆{1, . . . , n}.

The form chosen may depend on the particular application. Simultaneous detection of many unrelated objects may be better cast in the SoP form where each term corresponds to one object type. On the other hand detection of objects belonging to heterogenous families may be better cast in the PoS form, where each term represents a separate class of SBEs to which independent conditions may apply.

The form chosen may also determine the dimensionality of the implementation. To implement a conjunctive operation by a signaling cascade a unique PIP is required for each level. Furthermore, in the absence of special accommodations, the uniqueness must be maintained across all conjunctions that are summed together. For instance, for the SBEs s₁, s₂, s₃, s₄, the function (s₁+s₂) (s₃+s₄) may require two PIPs and two CUs in the PoS form. On the other hand, in the SoP form s₁s₃+s₁s₄+s₂s₃+s₂s₄, five PIPs and eight CUs may be used if no further design optimization is carried out. Recall that each unique PIP requires a specific signal object/SBE pair. Moreover, each CU occludes binding sites on the target object. Hence, the form of the Boolean function may have practical implications and may affect feasibility in various embodiments.

Practically, in each of the standard forms, both positive and negative conditions are to be expected. In the SoP form, products of positive and negative terms may be necessary in cases, where a given object may be characterized by SBEs that it must have and by SBEs that are specific to other objects and must therefore be absent. For instance, presence of epithelial markers and absence of leukocyte markers is necessary to reliably identify bloodborne epithelial cells. In the PoS form, sums of positive and negative terms may be necessary in cases where a given object may be characterized either by the presence of an SBE or by the absence of the whole underlying class of SBEs. For instance, antigen presentation via the major histocompatibility complex (MHC) may trigger an immune response if either a foreign epitope is displayed or if the MHC is in some part incomplete.

The logical operators and the corresponding methods provided are sufficient to realize general Boolean functions in both standard forms. Implementations of functions in the SoP form follow directly from methods described for the AND and NEGATED IMPLICATION operators (see FIG. 12). Towards the implementations of functions in the PoS form, first, write all multiplicative terms

$${Σ}_{s ∈ M} s + {Σ}_{s ∈ N} \overline{s} as \overline{( ( \overline{{Σ}_{s ∈ M} s} ) {Π}_{s ∈ N} s )} ,$$

where M, N c. As such, the multiplicative terms take on the form of a double NEGATED IMPLICATION.

To implement a Boolean function in the PoS form as written above, proceed as with the standard NEGATED IMPLICATION. For a triplet of SBEs s₁, s₂, s₃∈B, realize the function

$$\overline{{s}_{1} \overline{{s}_{2}}} {s}_{3} ,$$

with four objects q₁, q₂, q₃, q₄∈Q. Let q₁, q₂ realize the function s̅₁̅s̅₁̅, i.e., q₁ produces an intermediate signal z₁ whose recognition leads to degradation of z⁴ by q². Let the third object q₃ recognize s₂ and constitutively degrade z₁. Finally, let the fourth object q₄ recognize s₃ and constitutively produce z₄. The computation of the function by the z₄ PIP can be validated as above. Moreover, extensions to include more terms and or exclude s₃, proceed in the same manner as above.

With this information, implementation of functions in the general PoS form is straight forward (see FIG. 13). Multiplicative terms made up of non-negated variables are implemented by a single cascade and the remaining terms are combined, by De Morgan's law, into a single negated summation of NEGATED IMPLICATION operators. In other words, the general form is implemented as

$$( {Π}_{s ∈ L} s ) \overline{( {Σ}_{ι} ( \overline{{Σ}_{s ∈ {M}_{ι}} s} ) {Π}_{s ∈ {N}_{ι}} s )} ,$$

where, for i=1, 2, . . . , Mᵢ,Nᵢ⊆B.

### Canonical Form Omissions

The canonical form is intended for capturing logical behaviors. Consequently, some underlying physical behaviors are not directly considered. For instance, many complex phenomena are approximated with Boolean functions while, in many embodiments, signal recognition is highly dependent on the number of signal objects in the medium. SBE recognition is also assumed to be automatic while, in practice, this processes is driven by diffusion or agitation and is highly dependent the sizes of the SBE marked objects. Diffusion constraints may also inhibit signaling between co-localized objects in sufficiently large clusters. Moreover, in some embodiments, inadequate separation of different clusters may permit signal transmission between clusters. The canonical form also describes only the most essential mechanisms of internal state transition. In many embodiments, state transitions may be conditioned on deterministic or stochastic processes that may lead to signaling events happening in an unexpected order. Some operational precautions by which significance of such behaviors can be minimized are described as part of system operation.

The canonical form is also not intended for capturing the full spectrum of enabled functionalities. In some embodiments, more subtle design elements may be implemented. The canonical form does not consider cardinality of signal producing or signal degrading objects in a given cluster. In some embodiments, the functionality of these objects may be conditioned on the cardinality so as to avoid erroneous processing. The canonical form also does not consider copy numbers of various object associated entities. For instance, the SBE copy number is not considered. While in many embodiments, the copy number is sufficiently high to neglect its effects, in many embodiments some objects may intentionally comprise a fixed number of SBEs. Such objects may serve to relabel, block, or amplify SBEs on other objects. Computational processes considered by the canonical form also do not explicitly list mechanisms for processing target objects that are characterized by both the SBEs they display and the signals they produce. In some embodiments, however, signals produced by target objects may be considered by the device to improve the computation. Therein, the signals may be released but localized to the target object, or they may be tethered to the target objects, or they may be released throughout a medium comprising the system to globally condition the system computation. Global computations are another aspect not considered by the canonical form. In some embodiments, such computations may be considered a priori and subsequently used to instantiate the system objects. In some embodiments, however, background signal processing may be important for management of system level processes, e.g., management of signals transmitted away from object clusters in order to minimize unwanted cross-talk or to amplify local transmission. The canonical form also does not directly consider CU production. CU production may be important in signal amplification, dynamic computation, etc. The canonical form considered CU production only indirectly with lumped models wherein the functions of the newly produced CUs are grouped with the functions of the parent. For instance, a CU that produces signal degrading CUs is described by a lumped CU that degrades signals.

The canonical form is also not intended to capture limitations of the invention associated with certain physical constraints. Two limitations, time and space, are likely to affect many embodiments. With regards to time, two constraints are possible, computational time as prescribed by the application and operational time that is supported by the system setup. The computational time will in large part be determined by the depth of the signaling networks. Configurations wherein multilayered signaling is necessary to arrive at either a positive or a negative answer may be too time consuming for practical use. Operationally, many embodiments comprise objects with active metabolisms that must be maintained for correct function. Some configurations may not provision for endless supply of fresh medium and their operational time will be limited accordingly. Some embodiments also comprise objects that undergo natural replication cycles or have finite life expectancy. This will again lead to operational constraints. In combination, the computational time and the operational time may make some computations infeasible from a practical perspective. Nonetheless, it is expected that developments will follow the disclosure of this invention that will relax both the time and spatial constraints. For instance, embodiments implementing smaller computing units are possible. In the case of cellular CUs, genetic engineering may be used to produce smaller computing units. New embodiments comprising smaller species may also be proven in practice. Time constrains may be overcome by continuous monitoring of mixture conditions and by implementing faster signaling mechanisms.

### System Operation

The invention provides methods for implementing the computing device. These methods are organized in a computing architecture that casts broad terms from computer science into the device domain (see FIG. 14). Standard computer architectures (e.g., von Neu-mann or Harvard architectures) are widely used to organize operations of stored-program electronic digital computers. Similarly, the provided architecture and its implementations formulate and organize operations of PIC devices. This includes methods that filter device inputs, facilitate data reads, synchronize computing operations, and access device readouts. This also includes additional elements that standardize inputs, protect signal integrity, insulate distributed computations, and coordinate distributed operation to establish device readouts. In the remainder of this section, the computing architecture and relevant embodiments are disclosed.

### Object Pre-Treatment

Objects within the collected sample or within the set of computing entities may be pre-treated prior to the computing processes. Pre-treatment may slow or promote metabolic processes through external influence (e.g., temperature change) or chemical treatment (e.g., metabolite or inducer supplement). Pre-treatment may also expose or conceal object surfaces. In some cases, SBEs may be occluded by nonspecific layers (e.g., polysaccharide, glycoprotein layers). Such layers may be removed by appropriate enzymatic or chemical pre-treatment. In other cases, objects may comprise background entities that hinder recognition of target objects. Erroneous clustering with background objects may be minimized by pre-treatment of system objects with elements that interact non-specifically with surface entities and thereby fill the residual binding capacity. The so-called blocking entities should inhibit non-specific binding (passive and covalent) between SBEs or between surfaces, exhibit no cross-reactivity with SBEs, and should not disrupt the system objects.

### Input Relabeling

In the canonical form, SBE recognition is taken to be binary and SBE binding capacity was taken to be unbounded. In practice, different cognate pairs may exhibit different affinities and the size of target objects may limit the number of associated CUs. Such physical constraints may be irrelevant, e.g., in cases where SBE associations are highly specific and low order propositions are used. In various embodiments, however, it is important to balance cluster formation through input relabeling. For example, consider the case where SBE association is irreversible and described by first order binding kinetics. Let a single target object q₀ display SBEs s₁, s₂, . . . , sₙ that are uniquely recognized by objects q₁, q₂, . . . , qₙ at association rates k₁, k₂, . . . , kₙ, respectively. Form the corresponding mixture and proceed with clustering of the objects. Mathematical prediction suggests that the example will yield a cluster, where the relative representation of object qᵢ is given by

$$\frac{{k}_{i}}{k} ,$$

i=1, 2, . . . , and k=Σᵢ₌₁ⁿkᵢ. Furthermore, if all objects are approximately spherical with radius ρ, the target object will support at most 12 other objects (i.e., greatest integer less than 4π). Hence, even if n=2, a single order of magnitude difference in binding affinities may generate partial clusters. Relabeling of SBEs may obviate such biases assuring a higher percentage of complete clusters.

By relabeling inputs, variety in SBE display (e.g., naturally occurring SBEs in the collected sample) is normalized. Ideal labels for normalization should have the following properties. First, the binding kinetics of the transposed SBEs should be similar (e.g., covalent or non-covalent, similar chemistry). Second, copy numbers of SBEs localized to the same object should be similar. Third, the SBE transposition should be surjective (i.e., all SBEs should be labeled but no superfluous labels should be introduced). Lastly, the SBE labels should promote recognition by other objects. In some embodiments, additional features may be introduced. e.g., the labels may enable cluster isolation, cluster fragmentation, SBE detection by other SBEs, etc.

Within the context of the invention, labels are CUs comprising multiple SBE with various specificities. Such labels may be multispecific molecular entities with possible multivalency in some domains. Chemical or biological processes may be applied for SBE conjugation. The linker domains introduced in this process may be further optimized for length, inertness, rigidity, etc. The linker domain may also be extended to provide further computing functionality (e.g., programmable dissociation) or effector functionality (e.g., payload attachment).

### Object Clustering

Thus far, SBE recognition was assumed to be automatic. In some embodiments, a clustering reaction (see FIG. 14) may be used to ensure SBE recognition is completed. In various embodiments, object sizes (e.g., cellular CUs) are on the order of micrometers. At these scales, exogenous forces may be necessary to achieve sufficient mixing of objects. A 1 μm particle diffuses 1000 times slower than a 1 nm particle. The clustering reaction may use other mechanisms besides diffusion to promote interaction between SBEs. At the same time, the clustering reaction must minimize the effects that these mechanisms have on signaling. Forced interaction between objects resembles object co-localization, which is the basis for most computational operations. Hence, the clustering reaction must also implement mechanisms that prevent signaling between objects.

The invention provides a clustering method that simultaneously forces interaction and blocks signaling. During the clustering reaction, system objects (e.g., the collected sample and the computing entities) are combined (see FIG. 14). Combination of objects may be done all at once or progressively. In some embodiments, the reaction commences with resuspension of objects in a medium optimized for specific clustering (e.g., low viscosity, high ion content, containing blocking agents or solubility agents). The medium may also contain signal blockers (e.g., binding compounds or signal degrading enzymes) or metabolic suppressors (e.g., translation inhibitors). Alternatively, the medium may be depleted of essential metabolites (e.g., amino acids, carbon sources) required for high levels of signal production. In some embodiments, the reaction proceeds by forcing object interaction (e.g., hydrodynamic mixing, electromagnetic manipulation, mechanical compression). The choice of mechanism may depend on object properties. Hydrodynamic mixing may be appropriate in mixtures comprising objects of various masses or size. Electromagnetic manipulation is only applicable in mixtures where target objects or CUs are magnetizable. Objects may be magnetized by linkage (e.g., covalent or non-covalent bonding) to magnetizable entities or by loading (e.g., by electroporation, diffusion, carrier particles) the objects with magnetizable particles. Mechanical compression (e.g., by centrifugation) requires no special properties but may be more prone to erroneous processing. In all cases, the clustering reaction may be performed in a one-time batch process, continuously throughout the computing process (e.g., in a reactor), or repeated at multiple times during the computing process. In each execution, the results of the reaction (see FIG. 14) are clusters that co-localize CUs.

### PIP Evaluation

Thus far, the process of signal production was considered on a binary level (either signal is transmitted or signal is not transmitted). In some embodiments, the signaling processes (e.g., signal production, signal degradation, signal recognition, state transition, etc.) are mediated within a reaction optimized for signal evaluation (see FIG. 14). The evaluation reaction may initiate or terminate signal production and regulate chemical or spatial conditions. The primary goal is to protect cluster integrity and to ensure completion of relevant physiological and enzymatic processes.

In some embodiments, the evaluation and clustering reactions occur simultaneously. In other embodiments, the evaluation reaction is explicitly initiated following the clustering reaction. In these cases, the two reactions may share the same medium but remain separate through changes in temperature, object density, or illumination. In yet other embodiments, separation of the two stages may include a medium exchange. Whereas the medium of the clustering reaction is optimized for interaction (e.g., low viscosity, increased ion content, added blocking entities and solubility agents), the medium of the evaluation reaction may include enriched signaling precursors. The medium may be enriched in metabolites that support signal production and signal recognition. For instance, synthetic growth media may be optimized for CU metabolisms and enriched with inducers of wild-type or recombinant cellular systems. In addition, the properties of the media (e.g., pH, salt content) may be optimized for buffering and for any extracellular enzymatic processes (e.g., proteolysis, hydrolysis).

Spatial arrangement of the evaluation reaction may affect diffusion. Convective diffusion weakens signal strength requiring more sensitive SBEs that can be more error-prone. Hence, the evaluation reaction may take precautions to limit object motion. For instance, medium viscosity may be increased by addition of certain polysaccharides or other polymers. In addition, the additives may be crosslinked (e.g., by chemical amalgamation, ionic coupling, thermal, or photoinitiated crosslinking) forming a structured matrix that traps and immobilize system objects. The end result is greater signal accumulation and decreased signal transmission between clusters.

In some embodiments, according to any of the systems described herein, the system further comprises an agent that increases the viscosity of a medium comprising the system. In some embodiments, the agent is a polymer. In some embodiments, the polymer is a polysaccharide. In some embodiments, the agent is cross-linked to form a matrix configured to immobilize one or more of the system components.

### PIP Regulation

The evaluation reaction integrates three primary functions, PIP computation, PIP regulation, and PIP readout (see FIG. 14). The composition of the computational elements executing logical operations (e.g., AND, OR, NOT, and combinations thereof) was discussed above. In addition, regulatory elements may be added to the composition to improve the computational speed, correctness, or sensitivity.

Regulatory elements may act in cis by generating signal objects to increase the on-target/off-target signal ratio. For a pair of SBEs, s₁ and s₂, consider the provided realization of the AND operation s₁s₂. Recall that a target object q₀ is recognized by two CUs q₁ and q₂, where q₁ constitutively produces the signal intermediate signal z₁ that induces a change to a high state in q₂. To this system, the following regulatory mechanism may be added. Extend the state space of q₁ to include both low and high states, i.e., X_{q1}={x_{1L}, x_{1H}}, where only x_{1H} leads to production of z₁. Next, let q₂ produce the signal z_{2L} in the low state x_{2L}. The signal z_{2L} is recognized by q₁ and induces the transition from x_{1L} to x_{1H}. Now compare this regulated system to the unregulated system. Construct a truth table that maps the differences in signal production for both CUs q₁ and q₂. In the unregulated system, the PIP associated with q₁ remains the same for all target objects marked by s₁. Hence, the result of the AND operation is determined solely by q₂. On the contrary, for the regulated system, both q₁ and q₂ must confirm the logical output. This redundancy may improve the specificity and sensitivity of the result. Moreover, implementation of low signals decreases overall signal accumulation in the medium. Furthermore, induced signal production extends the operational range so that higher signal strengths may be used increasing convergence rate.

Regulatory elements may act in trans to maintain signal strength in higher order cascades (see FIGS. 15A and 15B). While the canonical form supposes binary signal production, in practice, signal production is continuous. Hence, in a cascade where the first object induces signal production in the second object, etc., the signal may be attenuated in each step yielding low sensitivity in the end. In such cases, additional objects may be introduced to regulate the signal strength. These objects may act as complements to objects already present. For the above example, consider the object q₂. The complement to q₂ is an object that recognizes the same SBE (i.e., s₂) but is otherwise identical to the object q₁. In other words, the complement to an object recognizes the same SBE as that object, is induced by the signal produced by that object, and, following induction, produces the signal recognized by that object. Such trans acting control elements ensure that signal strength is independent of the cascade size.

The compositions of regulatory elements provided above address signal production. Regulatory elements may also act in cis to regulate signal recognition. Such regulated systems, may establish quorum requirements in signaling, i.e., multiple signal producing CUs are required for affirmative signal recognition by co-localized signal recognizing CUs. Quorum requirements increase system reliability. For example, consider a well-mixed volume V comprising N objects that produce a given signal and one other object that recognizes that signal. In this example, the probability of no erroneous signal recognition events if m signal producers are required for induction is (1−(dV/V)^{m})⁽^{ₘ}^{^{N}}⁾. Hence, in a 1 ml volume, the probability of error-free transmission between 10 million objects and nanoliter sized clusters is almost zero. However, if the number of signal producing objects required for induction is increased to three (i.e., m=3), the probability becomes practically unity. Hence, even minor changes in signal recognition sensitivity may significantly reduce signaling errors.

Regulatory elements may also act in a target nonspecific manner. Such regulatory elements may comprise objects whose only function is signal degradation. In other words, to prevent transmission of the signal z₁ between clusters, introduce objects that recognize no SBEs and degrade z₁. This type of control is especially important in computing compound Boolean functions as signaling between objects may generate false positive results. For instance, in computing s₁ s₂, signal transmission from clusters displaying only s₁ to clusters displaying only s₂ may generate a false positive result.

### PIP Readout

In some cases, system readouts may be used to make measurement of PIP more convenient. System readouts comprise two general types, highly specific local readouts and global readouts with limited specificity. Local readouts are generated by individual clusters. For the practical reasons, local readouts must be highly specific in order to permit measurement by other analytical systems (e.g., PCR, ELISA). Signal objects may be specifically produced for this purpose. In some embodiments, local readouts may be based on production of specific polymer sequences (e.g., nucleic acids) that are either synthesized by cluster CUs or directly derived from cluster objects. Standard methods of polymer amplification may be thereafter used for measurement.

Similar to local readouts, global readouts are initially generated by individual clusters. Unlike local readouts, however, global readouts may involve secondary CUs that are not specific to the original cluster. Such CUs may be molecular entities capable of affecting quantitative or qualitative measurements. These include fluorescent proteins (e.g., GFP, RFP, YFP, CFP), luminescent proteins (e.g., luciferase, enzymes (e.g., beta-lactamase, beta-galactosidase, SEAP), antibody fragments, nucleic acids. Cellular CUs may also provide reporter functions by producing cytoplasmic reporter entities or by secreting reporter entities into the medium. In either case, the methods of evaluation include but are not limited to quantification of external signals based on their spectrophotometric or fluorometric properties, quantification of external signals based on bulk properties of the test volume (e.g., changes in color, luminescence), quantification based on detection of macroscopic aggregates, e.g., clusters formed by agglutination of entities in the sample or device, quantification of nucleic acid sequences.

### Methods

In one aspect, provided herein is a method of detecting the presence or absence of a target object in an input sample comprising: a) incubating a composition comprising a system according to any of the embodiments described herein suspended in a medium for a sufficient amount of time for the output signal to be generated; and b) detecting the output signal, thereby detecting the presence or absence of the target object in the input sample. In some embodiments, the medium is a liquid or a gas. In some embodiments, the medium is an aqueous medium. In some embodiments, the input sample is a biological sample derived from an individual. In some embodiments, the target object is a target cell. In some embodiments, the target cell is a disease cell (e.g., a cancer cell), a fetal cell, or a stem cell. In some embodiments, the target cell is a state-specific target cell (e.g., an activated leukocyte, an anergic leukocyte, a metabolically active/inactive cell, and the like). For example, the method may allow for the specific detection of activated leukocytes in an input sample as compared to quiescent leukocytes. In some embodiments, the target object is a target pathogen. In some embodiments, the target object is indicative of a disease or condition in the individual. In some embodiments, the input sample is an environmental sample (e.g., soil, dirt, sewage, air, water, plant material, and the like). In some embodiments, the input sample is a synthetic sample. In some embodiments, the target object is a contaminant (e.g., a foodborne pathogen or a biohazard). In some embodiments, the method further comprises quantifying the level of the target object in the input sample.

In another aspect, provided herein is a method of quantifying the level of a target object in an input sample comprising: a) incubating a composition comprising a system according to any of the embodiments described herein suspended in a medium for a sufficient amount of time for the output signal to be generated; and b) detecting the output signal, thereby quantifying the level of the target object in the input sample. In some embodiments, the medium is a liquid or a gas. In some embodiments, the medium is an aqueous medium. In some embodiments, the input sample is a biological sample derived from an individual. In some embodiments, the target object is a target cell. In some embodiments, the target cell is a disease cell (e.g., a cancer cell), a fetal cell, or a stem cell. In some embodiments, the target cell is a state-specific target cell (e.g., an activated leukocyte, an anergic leukocyte, a metabolically active/inactive cell). In some embodiments, the target object is a target pathogen. In some embodiments, the target object is indicative of a disease or condition in the individual. In some embodiments, the input sample is an environmental sample (e.g., soil, dirt, sewage, air, water, plant material, and the like). In some embodiments, the input sample is a synthetic sample.

In another aspect, provided herein is a method of diagnosing a disease or condition in an individual comprising: a) incubating a composition comprising a system according to any of the embodiments described herein suspended in a medium for a sufficient amount of time for the output signal to be generated; and b) detecting the output signal, thereby diagnosing the disease or condition in the individual. In some embodiments, the medium is an aqueous medium. In some embodiments, the input sample is a biological sample derived from an individual. In some embodiments, the target object is a target cell. In some embodiments, the target cell is a disease cell (e.g., a cancer cell, such as a circulating tumor cell (CTC)), a fetal cell, or a stem cell. In some embodiments, the target cell is a colorectal cancer cell (e.g., a metastatic colorectal cancer cell). In some embodiment, the target cell is a breast cancer cell (e.g., a metastatic breast cancer cell). In some embodiments, the target cell is a state-specific target cell (e.g., an activated leukocyte, an anergic leukocyte, a metabolically active/inactive cell). In some embodiments, the target object is a target pathogen. In some embodiments, the target object is indicative of a disease or condition in the individual.

### Compositions

In one aspect, the present disclosure provides compositions comprising a system as described herein suspended in a medium. In some embodiments, the medium is a liquid or a gas. In some embodiments, the medium is an aqueous medium.

In some embodiments, provided herein is a composition comprising one or more (such as any of 2, 3, 4, 5, or more) computing units (CUs), wherein the one or more CUs are configured to interact with one or more (such as any of 2, 3, 4, 5, or more) sample objects derived from an input sample such that an output signal indicative of a characteristic of the input sample is generated. In some embodiments, the composition further comprises the one or more sample objects. In some embodiments, the composition is configured such that one or more computational clusters can be formed, wherein each computational cluster comprises, independently, one or more of the CUs in the composition. In some embodiments, one or more of the computational clusters further comprise, independently, one or more of the sample objects. In some embodiments, the output signal comprises a level of an output signal object (SO) in the composition. In some embodiments, the input sample is a biological sample derived from an individual. In some embodiments, the input sample is an environmental sample. In some embodiments, the input sample is a synthetic sample.

In some embodiments, any components of a composition described herein are formulated with acceptable excipients such as carriers, solvents, stabilizers, diluents, etc., depending upon the particular biological computing system. Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

### Kits

In one aspect, the present disclosure provides a kit that contains any of the above-described system components and compositions described herein. In some embodiments, the kit further includes instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (e.g., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this embodiment is a kit that includes a web address where the instructions can be viewed and/or from which the instructions can be downloaded. As with the instructions, this means for obtaining the instructions can be recorded on a suitable substrate.

The present disclosure has been described above with reference to specific alternatives. However, other alternatives than the above described are equally possible within the scope of the disclosure. Different method steps than those described above, may be provided within the scope of the disclosure. The different features and steps described herein may be combined in other combinations than those described.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

In addition, where features or aspects of the disclosure are described in terms of Markush groups, those skilled in the art will recognize that the disclosure is also thereby described in terms of any individual member or subgroup of members of the Markush group.

Any of the features of an alternative of the first through eleventh aspects is applicable to all aspects and alternatives identified herein. Moreover, any of the features of an alternative of the first through eleventh aspects is independently combinable, partly or wholly with other alternatives described herein in any way, e.g., one, two, or three or more alternatives may be combinable in whole or in part. Further, any of the features of an alternative of the first through eleventh aspects may be made optional to other aspects or alternatives. Although described above in terms of various example alternatives and implementations, it should be understood that the various features, aspects and functionality described in one or more of the individual alternatives are not limited in their applicability to the particular alternative with which they are described, but instead may be applied, alone or in various combinations, to one or more of the other alternatives of the present application, whether or not such alternatives are described and whether or not such features are presented as being a part of a described alternative. Thus, the breadth and scope of the present application should not be limited by any of the above-described example alternatives.

All references cited herein are incorporated herein by reference in their entirety. To the extent publications and patents or patent applications incorporated by reference contradict the disclosure contained in the specification, the specification is intended to supersede and/or take precedence over any such contradictory material. To the extent publications and patents or patent applications incorporated by reference herein contradict the disclosure contained in the specification, the specification is intended to supersede and/or take precedence over any such contradictory material.

The details of one or more embodiments of the disclosure are set forth in the accompanying description below. Any materials and methods similar or equivalent to those described herein can be used in the practice or testing of the present disclosure. Other features, objects, and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### EXAMPLES

### Materials and Methods

### Media and Buffers

Yeast Peptone Dextrose (YPD) media—100 ml: 1 g Bacto-yeast extract (VWR—s.n. VWRC84601.0500), 1 g Bacto-peptone (VWR—s.n. VWRC84620.0500), 2 g glucose (VWR—s.n. VWRC0188-1KG).

Synthetic Drop-out Complete (SDC) media—100 ml: 0.67 g Bacto-yeast nitrogen base (Merck—s.n. Y0626-250G), 2 g glucose (VWR—s.n. VWRC0188-1KG), 0.2 g drop-out complete AA mix, pH 6.5.

2×SDC+1% Sodium alginate—100 ml: 1.34 g Bacto-yeast nitrogen base (Merck—s.n. Y0626-250G), 4 g glucose (VWR—s.n. VWRC0188-1KG), 0.4 g drop-out complete AA mix, 1 g alginic acid sodium medium viscosity (Sigma—A2033-100G), pH. 6.5.

1×TBS—100 ml: 0.877 g NaCl (VWR—LACH30093APOG0500), 1 ml 1M Tris-HCl, pH 7.6 (Sigma—T2444-100ML).

1×TBS+1% gelatine—100 ml: 100 ml 1×TBS, 1 g gelatine (VWR—VWRC9764-500G).

### Background Strains

All strains are derived from the S. cerevisiae strains BY4741 aga2Δ (MATa; ura3Δ0; leu2Δ0; his3Δ1; lys2Δ0; YJR004c::kanMX4) and BY4742 sag1Δ (MATα; ura3Δ0; leu2Δ0; his3Δ1; met15Δ0; YGL032c::kanMX4). Mutants comprising gene deletions and promoter modifications were constructed using standard methods. In the case of a gene deletion, a barcode sequence encoding a pair of stop codons was introduced within the first 100 bases of the wild type ORF.

TABLE 1

| Gene | Position | Barcode |
|---|---|---|
| MFA1 | Chromosome IV | TGATTCGGTA ATGCGCTGTA A |
| | 1385179 . . . 1385199 | (SEQ ID NO: 4) |
| MFA2 | Chromosome XIV | TAACTAACT |
| | 352417 . . . 352474 | (SEQ ID NO: 5) |
| MFc1 | Chromosome XVI | TAATACGCGA AACTCCAGTC G |
| | 194069 . . . 194089 | (SEQ ID NO: 6) |
| MFc2 | Chromosome VII | TAATCAGGGA AAGATCCTTC G |
| | 344842 . . . 344863 | (SEQ ID NO: 7) |
| BAR1 | Chromosome IX | TAAACTGAGC TGACCTAAGG G |
| | 322414 . . . 322434 | (SEQ ID NO: 8) |
| PTP3 | Chromosome V | TGATGACCCA CTCAGACATG A |
| | 308446 . . . 308467 | (SEQ ID NO: 9) |
| STE2 | Chromosome IV | TAGTTGGCCCACGAACCGTAA |
| | 82583 . . . 82603 | (SEQ ID NO: 10) |
| STE3 | Chromosome XI | TGATTCGGTAATGCGCTGTAA |
| | 114576 . . . 114556 | (SEQ ID NO: 11) |
| FAR1 | Chromosome X | TCCGGATGACACTCGACGCCATAGAT |
| | 126297 . . . 126272 | (SEQ ID NO: 12) |
| SST2 | Chromosome XII | TGAAAGATCAAGTTGTAGGCC |
| | 1041315 . . . 1041295 | (SEQ ID NO: 13) |

Promoter modifications comprised placing the putative constitutive promoter pREV1 (Chromosome XV 981128 . . . 981827) followed by the Xhol restriction site (AGATCT) upstream of the wildtype ORF. In some cases, the promoter was further insulated by the terminator tTDH1 (Chromosome X 339270 . . . 339494) and a dummy sequence dURA3 (Chromosome V 116078 . . . 116676). The modifications are detailed in Table 2 below.

TABLE 2

| Gene | Deleted sequence | Inserted sequence |
|---|---|---|
| SST2 | none | dURA3-tTDH1-pREV1 |
| MSG5 | Chromosome XIV | pREV1 |
| | 529644 . . . 529941 | |

Strain rsX291 was modified by replacing a sequence of the GPA1 gene (chromosome VIII, 114903 . . . 114917) encoding residues 468-472 with the sequence GAATGTGGTCTGTAC (SEQ ID NO: 14), encoding the last five amino acids of human GNAI3.

The following is an indexed list of background strains used in the construction of various CUs described herein.

TABLE 3

| ID | genotype |
|---|---|
| sX111 | BY4742 sag1L mfc1L mfc2L |
| sX127 | BY4741 aga2L bar1L mfa1L mfa2L |
| sX357 | BY4742 sag1L mfc1L mfc2L |
| | pMSG5::pREV1 pSST2::pREV1 |
| sX373 | BY4741 aga2L bar1L mfa1L mfa2L ptp3L pMSG5::pREV1 |
| rsX291 | BY4741 aga2L bar1L mfa1L mfa2L mfx1L mfx2L |
| | sst2L ste2L ste3L far1L GPA1(468-472)::ECGLY |

### Gene Cassettes

Cassettes were constructed for the purposes of CU development (see Table 4 below for an indexed list). Cassettes are bacteria/yeast shuttle vectors bearing a bacterial marker (AmpR or KanR) together with the origin of replication ColE1. The cassettes mediate integration into the yeast genome via a pair of homologous regions (Homologous Left and Homologous Right) that target recombination to a specific locus (ura3, his3, leu2, lys2, met15). The cassettes further include auxotrophic markers for selection of transformed clones. Inserts are cloned into the cassettes using standard methods.

TABLE 4

| | | Selection |
|---|---|---|
| ID | Function | Marker |
| cSBE | SBE display cassette | URA3 |
| cEXP-L | Pheromone and receptor expression | LEU2 |
| cEXP-M | Pheromone expression | MET15 |
| cEXP-S | Pheromone expression | LYS2 |
| cEXP-H | Reporter and Bar1 expression | HIS3 |
| cEXP-U SO | Signal object expression | URA3 |
| cMG | Multi-gene expression | LEU2 |

The SBE display cassette (cSBE, see FIG. 19) enables anchoring of peptides to the cell wall. The insert encodes the SBE. In the final construct, the ORF begins with the Ost1 Pre sequence (initial 22 AA residues of YJLOO2C ORF, codon optimized for expression in yeast S. cerevisiae) followed by the MF(alpha) Pro sequence (AA residues 19-89 of YPL187W ORF, codon optimized for expression in yeast S. cerevisiae). The ORF continues with the insert sequence followed by a rigid extension linker (sequence A(EAAAK)₃A, SEQ ID NO: 2, codon optimized for expression in yeast S. cerevisiae) and a truncated version of FLO11 (last 1135 AA residues of YIR019C ORF). The gene is expressed from the promoter pTDH3 (first 700 bases upstream of the YGR192C ORF). The cassette mediates integration into the ura3 locus and selection with the URA3 auxotrophic marker.

The expression cassettes (cEXP-L, cEXP-M, cEXP-S, cEXP-H, see FIG. 20) enable tunable expression of various products. The insert is a gene comprising a promoter and an ORF. In the final construct, the gene is placed upstream of the terminator tTDH1. The various versions of this cassette use different auxotrophic markers and homologous regions, as described in Table 4 above.

The cEXP-U SO expression cassette enables inducible expression of peptidic signal objects. The insert encodes the signal object. In the final construct, preceding the expressed ORF, a TetR (Escherichia coli TETR2 ORF codon optimized for expression in S. cerevisiae) is expressed under the control of pRPL18B (first 700 nucleotides of YNL301C ORF). The signal object ORF is expressed under the control of an inducible pTet promoter (SEQ ID NO: 15). The ORF begins with the MF(ALPHA)1 prepro sequence (AA residues 1-89 of YPL187W ORF). The ORF continues with the insert sequence followed by a stop codon. The cassette mediates integration into the ura3 locus and selection with the URA3 auxotrophic marker.

The multigene cassette (cMG, see FIG. 21) enables one-step integration of multiple gene constructs into a single locus. The cassette includes three insert domains. Two inserts correspond to pheromone ORFs, where the first is expressed from the PSP2 promoter (a medium strength constitutive promoter) and the second is expressed from the pheromone inducible FIG. 1 promoter. The third insert corresponds to the SBE ORF, where the context of the SBE is the same as in the SBE display cassette. In addition to the three variable genes, the cassette includes the BLA reporter gene described in more detail below. The cassette mediates integration into the 1eu2 locus and selection by the LEU2 auxotrophic marker.

### Gene Constructs

The above cassettes were used to make the following gene constructs that are described in detail below.

TABLE 5

| ID | Insert | Vector |
|---|---|---|
| gX262 | FLAG9 | cSBE |
| gX325 | HEMA9 | cSBE |
| gX219 | pRAD27-MF(alpha)1 | cEXP-S |
| gX223 | pRNR2-MF(alpha)1 | cEXP-S |
| gX224 | pRPL18B-MF(alpha)1 | cEXP-S |
| gX226 | pTDH3-MF(alpha)1 | cEXP-S |
| gX193 | pFIG1-BLA | cEXP-H |
| gX328 | pTDH3-BAR1 | cEXP-H |
| rgX007 | pTet-MFW-C5A | cEXP-U SO |
| rgX012 | pTet-MFW-NTS | cEXP-U SO |
| rgX014 | pTet-MFW-SST-14 | cEXP-U SO |
| rgX197 | pTEF1-MFW-NTSR1 | cEXP-L |
| rgX219 | pTEF1-MFW-C5AR1 | cEXP-L |
| rgX379 | pTEF1-MFW-SSTR2 | cEXP-L |
| mX001 | MFA1/MFA1/HEMA9 | cMG |
| mX002 | MF(alpha)1/ | cMG |
| | MF(alpha)1/ | |
| | FLAG9 | |

The SBE FLAGS comprises three 3×FLAG domains (DYKDHDGDYKDHDIDYKDDDDK, SEQ ID NO: 1) joined by rigid linkers (A(EAAAK)₃A, SEQ ID NO: 2). The SBE HEMA9 comprises nine HA tags (YPYDVPDYA, SEQ ID NO: 3) organized into three domains joined by rigid linkers (A(EAAAK)₃A, SEQ ID NO: 2), where the individual domains have the form HA tag/(SG4)2/HA tag/(SG4)2/HA. The ORF maps are also shown in FIG. 22.

Pheromone producing genes comprise either the MFA1 (YDR461W) or MF(alpha)1 (YPL187W) ORF downstream of either a constitutive or an inducible promoter. The constitutive promoters (pPSP2, pRAD27, pRNR2, pRPL18B, pTDH3) comprise the first 700 bases upstream of the respective ORFs (YML017W, YKL113C, YJL026W, YNL301C, YGR192C, respectively). The pheromone inducible promoter pFIG. 1 comprises the first 500 bases upstream of the ORF YBR040W.

The gene (pFIG1-BLA) produces the enzymatic reporter beta-lactamase that hydrolyzes the beta-lactam ring and may be used to generate visible readout signals. The construct comprises the beta-lactamase ORF (obtained from the vector pUC19) fused to the FLP signaling sequence (first 63 bases of YIR019C) and expressed from the pFIG. 1 promoter.

The gene pTDH3-BAR1 produces an aspartyl protease that is secreted into the periplasm, where it degrades alpha pheromone. The gene comprises the YIL015W ORF downstream of the pTDH3 promoter.

The genes rgX007, rgX012, and rgX014 produce recombinant human signal objects (C5a, NTS, and SST-14, respectively) that are secreted by the cell. The genes contain the respective sequences encoding complement component 5a (C5a), neurotensin (NTS), and SST-14, codon-optimized for expression in S. cerevisiae expressed from the cEXP-U SO cassette.

The genes rgX197, rgX219, and rgX379 produce recombinant human receptors (NTSR1, C5AR1, and SSTR2, respectively) that are integrated into the plasma membrane. The gene comprises the ORF of the respective human gene placed downstream of the pTEF1 promoter (first 700 nucleotides preceding the YPR080W ORF) and an MF(ALPHA)1 prepro sequence (AA residues 1-89 of YPL187W ORF).

Multi-gene constructs were assembled from the same ORFs as above. In each case, two identical pheromone ORFs were placed into the pheromone cloning sites and the SBE coding sequence was placed into the SBE cloning site.

### Cellular Computing Units

Cellular CUs were constructed by homologous recombination of the gene constructs into the listed background strains.

TABLE 6

| ID | Background | Genes |
|---|---|---|
| sX128 | sX127 | pfX193 |
| sX137 | sX111 | gX226 |
| sX212 | sX111 | gX262 |
| sX216 | sX127 | gX193, gX262 |
| sX422 | sX127 | gX193, gX325 |
| sX403 | sX111 | gX219, gX262 |
| sX404 | sX111 | gX223, gX262 |
| sX405 | sX111 | gX224, gX262 |
| sX406 | sX111 | gX226, gX262 |
| sX417 | sX111 | gX226, gX325 |
| sX437 | sX127 | gX328 |
| sX444 | sX127 | gX262, gX328 |
| sX384 | sX357 | mX002 |
| sX387 | sX373 | mX001 |
| rsX324 | rsX291 | rgX197 |
| rsX389 | rsX291 | rgX219 |
| rsX420 | rsX291 | rgX219, rgX007 |
| rsX544 | rsX291 | rgX379 |
| rsX626 | rsX291 | rgX379, rgX014 |
| rsX769 | rsX291 | rgX197, rgX012 |

### Example 1: SBE Display Specificity

The specificity of SBEs toward their cognate ligands was tested in agglutination assays involving antibody labeled agarose beads. Agarose beads covalently linked to anti-FLAG M2 antibodies (A2220 Sigma-Aldrich) were used as a potential agglutination partner for FLAGS CUs (sX216). Agarose beads covalently linked to anti-HA antibodies (26182 Thermo Fisher) were used as a potential agglutination partner for HEMA9 CUs (sX422).

All strains were grown overnight in yeast peptone dextrose medium (YPD) and diluted into synthetic drop-out complete medium (SDC) to 0.05 OD600 4 hrs before experiment. Prior to experiment cells were washed once in tris-buffered saline (TBS) containing 1% gelatin, washed a second time in TBS, and resuspended in TBS to 0.01 OD600. Agarose beads were washed once in TBS and resuspended in TBS to a final concentration of 20,000 beads/ml.

In a 1.5 ml microcentrifuge tube, 90 μl of cell mixture (sX216 or sX422) was combined with 10 μl (approx. 100 beads) of bead solution in a 100 μl final volume. Agglutination was initiated by spinning down the mixture 7 times at 1000 rpm for 30 sec with resuspension between cycles. Beads were then visualized at 100× magnification. The results are shown in FIG. 23. Each CU type recognized its respective target with complete specificity. No recognition of non-specific targets was observed. In addition, no non-specific agglutination between CUs was observed.

### Example 2: Reporter Quantification

Rate of nitrocefin hydrolysis was used to quantify the concentration of BLA reporter in the medium. Samples were then spun down at 5000 rcf for 5 min and 90 ul of the supernatant was mixed with 10 μl of 1 mM nitrocefin (484400 Sigma-Aldrich) into a 96-well microplate. The beta-lactamase concentration was quantified in a microplate reader at 37 degC by measuring absorbance at wavelengths 486 nm (A486) and 700 nm₀700) every 2 minutes for 1 hour. One unit of BLA (1U) was defined as the amount of enzyme needed to degrade lnmol of Nitrocefin in 1 hour. In all experiments, the absorbance at 486 nm was normalized by subtracting the absorbance at 700 nm to compute absorbance difference (A) at each time instance

Δ(t)=A486(t)−A700(t) (1)

Substrate units denoted by _{Δ1U} were quantified by completely degrading 1 nmol of nitrocefin and measuring the maximal value of Δ(t). The factor Δ_{1U} was measured to be 0.03. The readout, i.e., beta-lactamase concentration, was then computed as the absolute change in A in the first 30 min normalized by the time and Δ_{1U}.

$$reporter = \frac{1}{{Δ}_{1 U}} \frac{Δ ( 30 min ) - Δ ( 0 min )}{30 min} [ U ] \qquad ( 2 )$$

### Example 3: Signaling Between Cellular CUs

Signaling between cellular CUs was demonstrated in a series of assays characterizing the dependence of signal transmission on the relative abundance of signal producing and signal recognizing CUs. In this study, the ratio of signal producing CUs to signal recognizing CUs was varied between 0 and 1 while the overall concentration of CUs was maintained at 0.05 OD600. This setup reflects CU signaling within an object cluster where the overall number of objects is constrained by cluster geometries but the relative representation of each object type may vary with the mixture composition.

All strains were grown overnight in YPD and diluted into SDC to 0.05 OD600 4 hrs before experiment. Prior to experiment cells were washed once in TBS containing 1% gelatin, washed a second time in TBS, and resuspended in TBS to 0.1 OD600. In each set of experiments, a signal producing strain (sX212, sX403, sX404, sX405, or sX406) was mixed in a 1.5 ml microcentrifuge tube with the signal recognizing strain sX128 at the ratios 1:9, 3:7, 6:4, and 8:2. The mixture was then further diluted with 2 times concentrated SDC (2×SDC)+1% Sodium alginate (Sigma—A2033-100G) to a final 0.05 OD600 in a 100 μl final volume. The reaction mixture was than incubated at 30° C. for 3 hours in a Bio RS-24 rotator (Biosan) at 4 rpm. Following the incubation period, all mixtures were spun down at 5000 rpm for 2 min and 90 μl of supernatant was transfered into a 96-well plate for nitrocefin-BLA analysis. The results are shown in FIGS. 24 and 25, where FIG. 24 corresponds to weak signal producers (sX212, sX403, sX404) and FIG. 25 corresponds to the strong signal producers (sX404, sX405, sX406). The medium signal producer sX404 is included in both figures for reference. The data is further fitted with a mathematical model of signal transmission as described below. Data is shown to be in good agreement with the model. It is shown that the optimal ratio (marked by the peak of the associated readout curve) of signal producing to signal recognizing CUs varies nonlinearly with strength of signal production. At weak signal production, the optimal ratio (approx. 0.6) calls for a greater fraction of signal producing CUs. At intermediate signal production, the optimal ratio remains relatively unchanged. Only when signal production is significantly increased does the optimal ratio shifts in the direction of signal recognizing CUs.

### Example 4: Signal Degradation by Cellular CUs

Inhibition of signaling by cellular CUs was demonstrated in a series of assays characterizing the dependence of signal transmission on the relative abundance of signal degrading CUs. Three types of CUs were considered: signal producing CUs, signal recognizing CUs, and signal degrading CUs. The total concentration and relative abundance of signal producing and signal recognizing CUs was held fixed. The abundance of signal degrading CUs was varied between 0 and 2 relative to the signal producing CUs. This setup demonstrates that signaling can be completely inhibited with sufficient representation of signal degrading CUs in the object cluster.

All strains were grown overnight in YPD and diluted into SDC to 0.05 OD600 4 hrs before experiment. Prior to experiment cells were washed once in TBS+1% gelatin, washed a second time in TBS, and resuspended in TBS to 0.04 OD600. In each set of experiments the signal producing strain sX406 and the signal recognizing strain sX128 were mixed in a 1.5 ml microcentrifuge tube at ratio of 1:1 in a total volume of 25 μl. Subsequently, the strain mixture was further combined with the signal degrading strain sX437 at various sX437 to sX406 ratios (2:1, 1:1, 1:2, 1:4, 1:8, 1:16, 1:32, and 1:64) in a total volume of 50 μl. The mixture was then further diluted with 2 times concentrated 2×SDC+1% Sodium alginate to a 100 μl final volume. The reaction mixture was incubated at 30° C. for 3 hours in a Bio RS-24 rotator (Biosan) at 4 rpm. Following the incubation period, all mixtures were spun down at 5000 rcf for 2 min and 90 μl of supernatant was transferred into a 96-well plate for nitrocefin-BLA analysis. The results are shown in FIG. 26. It is shown that even a weak representation of signal degrading CUs (approx. 5% relative to signal producing CUs) is sufficient for 5-fold signaling inhibition.

### Example 5: Mathematical Model of Signaling

A mathematical model is proposed to provide a mechanistic representation for signal transmission within a heterogeneous population of CUs. The model considers a mixture comprising producer CUs and reporter CUs. Producer CUs constitutively secrete signal objects into the medium. Reporter CUs recognize the signal objects and producer reporter signals in accordance with the signal concentration.

The following notation is used.

TABLE 7

| Variable | Symbol |
|---|---|
| Number of producer CUs | N_{P} |
| Number of reporter CUs | N_{R} |
| Number of all units | N_{T} |
| Density of producer CUs | R |
| Signal secreted by producer CUs | α |
| Reporter signal secreted by reporter CUs | y |
| Effective production rate of signal α | c_{P} |
| Effective maximum production rate of y | K |
| EC50 of α | T |
| Hill coefficient of induction | H |

The mathematical model is derived as follows. All signal concentrations are taken at steady state and the CU copy numbers are assumed to be constant. Producer CUs secrete the signal a at an equal constitutive rate so that the effective production rate (production/degradation) is cₚ. The concentration of the signal in the medium is then given by

α=cₚNₚ (3)

Reporter CUs secrete the reporter signal y at a rate that depends on the concentration of α. The dependence is given by a hill function with the hill coefficient H and the EC50 T. The effective maximum production rate is given by K. The total concentration of y in the medium is then given by

$$y = {N}_{R} \frac{{K}_{{α}^{H}}}{{α}^{H} + {T}^{H}} \qquad ( 4 )$$

Define the ratio R of producer CUs to the total number of CUs.

$$R = \frac{{N}_{P}}{{N}_{T}} = \frac{{N}_{P}}{{N}_{P} + {N}_{R}}$$

Subsequently, by substitution and lumping of terms, the following single equation models the dependence of y on R.

$$y = c ( 1 - R ) \frac{{R}^{H}}{{R}^{H} + {τ}^{H}} , \qquad ( 5 )$$

where

$$τ = \frac{T}{{c}_{P} {N}_{T}} , \\ c = K {N}_{T} .$$

Equation 5 was fitted to experimental data obtained in Example 3.

Of interest is the optimum value of R that gives the maximal reporter signal y*. The character of Equation 5 indicates that the maximum can be computed from the first derivative of Equation 5 with respect to R. Appending the equation

$$\frac{d y}{d R} = 0$$

to Equation 5 gives a two equation system that can be solved for the maximum value of y as a function of R.

$${y}^{*} ( R ) = c ( 1 - \frac{H + 1}{H} R ) \qquad ( 6 )$$

Hence, the maximum signal is a linearly decreasing function of R. In other words, in a CU population, where the total number of CUs and the kinetic parameters describing signal transmission are fixed, the relationship between the maximal signal and the mixture composition is given by the linear function in Equation 6. To move along this line, one can change the production rate of a.

### Example 6: Pheroimmuno Computing Assay in Liquid Medium

The following protocol was used in PIC experiments illustrating various logical operations. This protocol is an embodiment of the PIC architecture wherein the mixture objects are permitted mobility in the medium.

In some embodiments, computation is performed on antibody labeled beads, where the beads serve as target objects with known SBE profiles. Three different classes of beads are considered to demonstrate the basic logical operations: agarose beads covalently linked to anti-FLAG M2 antibodies (A2220 Sigma-Aldrich) denoted by (BaF), agarose beads covalently linked to anti-HA antibodies (26182 Thermo Fisher) denoted by (BaH), and polystyrene beads 15 μm in diameter covalently linked to anti-HA and anti-FLAG M2 antibodies (custom production by Spherotech inc.) denoted by (BaHaF).

The following preparatory steps are implemented. Dextran beads (Sigma G2580) were washed once in TBS and diluted in TBS to the final concentration of 20,000 beads/ml. Wash agarose beads once in TBS and resuspend in TBS to a final concentration of 20,000 beads/ml. Store beads at 4° C. until use. Grow strains overnight in YPD and 4 hrs before experiment dilute into SDC to 0.05 OD600. Prior to experiment wash cells in tris-buffered saline (TBS)+1% gelatin, wash cells a second time in TBS, and resuspended pellet in TBS to 0.04 OD600.

The clustering reaction is prepared in a 1.5 ml microcentrifuge tube. Combine 90 μl of cell mixture (densities of individual strains are specified separately) with 10 μl (approx. 100 beads) of bead solution. Cluster objects by spinning down the mixture 7 times at 300 rcf for 30 sec. Resuspend pellet by light vortex between steps. This completes the clustering reaction.

The evaluation reaction continues in the same 1.5 ml tube. Dilute each clustering reaction with 2×SDC+1% Sodium alginate (Sigma-Aldrich A2033-100G) to a final volume of 200 Incubate the reactions at 30° C. for 3 hrs in a Bio RS-24 rotator (Biosan) at 4 rpm. Following the incubation period, collect the supernatant by spinning down the reactions at 5000 rcf for 2 min. Transfer 90 μl of supernatant into a 96-well plate for nitrocefin-BLA analysis.

In some cases, results are further interpreted in terms of fold-change. Fold-change is defined for all samples as the ratio of sample reporter amount and the negative control reporter amount, where the negative control is defined as the sample with no target objects.

### Example 7: Evaluation of Pheroimmuno Propositions by Simple Signaling Networks

In the following experiments, evaluation of pheroimmuno propositions (PIPs) is shown. In the presented embodiments a single SBE is transposed into signal objects of a single type. The essential feature demonstrated in this study is the localization of PIPs to the formed cluster. This is accomplished by coupling the PIPs with a reporter producing strain. It is also shown that regulatory CUs are necessary to discriminate positive samples from negative controls.

Three cellular CUs make up the system, the producer strain sX406, the reporter strain sX216, and the regulatory strain sX437. The producer strain constitutively secretes alpha pheromone into the medium. The reporter strain recognizes alpha pheromone and subsequently secretes the beta-lactamase reporter. Both of these strains recognize the same SBE, the anti-FLAG M2 antibody. The regulatory strain sX437 degrades the alpha pheromone. Unlike the other strains, the regulatory strain does not recognize any SBEs and so remains suspended in the medium. The schematic of the signaling network within the cluster is pictured in FIG. 10, where the two SBEs (1 and 2) are both of the same type (anti-FLAG M2 antibody) and the terminal output (9) is the reporter beta-lactamase. The schematic of the regulatory mechanism is shown in FIG. 17.

A set of experiments that evaluate the output for BaF target objects was executed to characterize the dependence of reporter concentration on mixture composition. In each experiment, all parameters were the same with the exception of the relative density of the producer strain to the reporter strain. Four mixture compositions were tested, with

$$\frac{s X 406}{s X 406 + s X 216} = 0.1 , 0.3 , 0.6 , or 0.8 \qquad ( 7 )$$

In addition, the regulatory strain sX437 was added to each mixture with its relative density (sX437/(sX406+sX216)) being 6%.

For each composition of CUs, the system was tested both with BaF target objects and, for negative control, without BaF target objects. The experiments were also repeated with the non-signaling version of the producer strain sX212, which recognizes the anti-FLAG M2 antibody but does not produce the alpha pheromone. All experiments were performed in triplicates.

The results are shown in FIGS. 27 and 28, where FIG. 27 corresponds to mixtures comprising the regulatory strain and FIG. 28 corresponds to mixtures where the regulatory strain is missing. Overall, the regulatory strain decreases reporter production. Moreover, however, the regulatory strain localizes signaling to clusters making affirmative PIPs discernible from basal reporter production in the background. The observed fold-change is approximately two fold.

### Example 8: Evaluation of Composed OR Operations

In the following experiments, evaluation of PIPs representing the logical operation OR is demonstrated. In the presented embodiments two different SBEs are transposed into signal objects of a single type. The essential features demonstrated in this study are the ability to consider multiple SBEs and the ability to compose independent computations within the same reaction. This is accomplished by multiplexing the signaling network presented above, by which a single PIP was evaluated.

Five cellular CUs make up the system, two producer strains (sX406 and sX417), two reporter strains (sX216 and sX422), and a regulatory strain (sX437). The producer strains secrete constitutively alpha pheromone into the medium. The reporter strains recognize alpha pheromone and subsequently secrete the beta-lactamase reporter. The first signaling pair (sX406 and sX216) recognize the anti-FLAG M2 antibody. The second signaling pair (sX417 and sX422) recognize the anti-HA antibody. The regulatory strain sX437 degrades the alpha pheromone and remains suspended in the medium. A partial schematic of the signaling network within the cluster is pictured in FIG. 9, where signal recognition and reporter production is omitted but remains the same as before. The schematic of the regulatory mechanism is shown in FIG. 17.

A set of experiments that evaluate the output for various target object inputs was executed and demonstrates the conformity of the system with the logical operator OR. Three independent experiments were performed. In each case the CU mixture was identical. The five strains sX406, sX417, sX216, sX422, and sX437 make up 27.35%, 27.35%, 18.25%, 18.25%, 8.8% of the total cell density, respectively. The experiments varied in the type of target objects included. Experiment A includes only BaF, Experiment B includes only BaH, and Experiment NC is the negative control where target objects are omitted altogether. The experiments were performed in triplicates and the results (see FIG. 29) are given in terms of measured fold-change as described in the PIC assay protocol. In all cases, a significant (3- to 4-fold) change between either Experiment A or B and the negative control was observed. This confirms that indeed the system computes the OR operation (e.g., anti-FLAG M2 OR anti-HA antibody).

### Example 9: Evaluation of and Operations

In the following experiments, evaluation of PIPs representing the logical operation AND is demonstrated. In the presented embodiments two different SBEs are transposed into two different signal objects. The essential feature demonstrated in this study is the ability to recognize and analyze a combination of SBEs on a single target object. This is accomplished by a cascade signaling network that is similar to the simple cascade demonstrated above.

Three cellular CUs make up the system, a producer strain (sX406 or sX137), a reporter strains (sX422 or sX128), and a regulatory strain (sX437). The producer strains constitutively secrete alpha pheromone into the medium. The reporter strains recognize alpha pheromone and subsequently secrete the beta-lactamase reporter. The regulatory strain degrades alpha pheromone. Schematics describing the underlying signaling and regulatory mechanisms are included in FIG. 10 and FIG. 17.

The alternate producer-reporter pairs represent different AND operators with the particular operator determined by the recognized SBEs. If the producer strain recognizes the set of SBEs {P1, P2, . . . } and the reporter strain recognizes the set of SBEs {R1, R2, . . . }, then the system implements the operator (P1 OR P2 OR . . . ) AND (R1 OR R2 OR . . . ). The following is the SBE assignment for this set of experiments. The producer strain sX406 recognizes the anti-FLAG M2 antibody. The reporter strain sX422 recognizes the anti-HA antibody. The producer strain sX137 and the reporter strain sX128 recognize a dummy SBE denoted by SBE-X, where SBE-X represents any specific entity (non-specific interactions are assumed to be negligible) other than the anti-FLAG M2 antibody or the anti-HA antibody (or an entity with similar epitope specificity). Hence, the pair sX406 and sX422 implements the operator anti FLAG M2 AND anti-HA, the pair sX406 and sX128 implements the operator anti FLAG M2 AND SBE-X, the pair sX137 and sX422 implements the operator SBE-X AND anti-HA, the pair sX137 and sX128 implements the operator SBE-X AND SBE-X.

The above producer-reporter computing systems were tested on the BaHaF target objects to show the conformity of the simple cascade signaling network with the AND operator truth table. Five independent experiments were performed. In each case a producer strain (sX406 or sX137), a reporter strain (sX422 or sX128), and the regulatory strain sX437 make up 46.3%, 46.3%, 7.4% of the total cell density, respectively. Identical BaHaF target objects were used in all cases with the exception of negative controls, where target objects are omitted altogether. The experiments were performed in triplicates and the results (see FIG. 30) are given in terms of measured fold-change as described in the PIC assay protocol. In all cases, outputs of systems implementing false propositions were 2- to 4-fold lower than the output generated by the system implementing the operator anti-FLAG M2 AND anti-HA.

### Example 10: Evaluation of NOT Operations

In the following experiments, evaluation of PIPs representing the logical operation NOT is demonstrated. In the presented embodiments two different SBEs are transposed into two different signal objects. The essential feature demonstrated in this study is the ability to negate the presence of an SBE in the presence of a different SBE. This is accomplished by composition of cascaded signaling and localized signal degradation.

Three cellular CUs make up the system, the producer strain sX406, the reporter strains sX216, and a inhibitory strain (sX437 or sX444). The producer strain constitutively secretes alpha pheromone into the medium. The reporter strain recognizes alpha pheromone and subsequently secretes the beta-lactamase reporter. The inhibitory strains degrade alpha pheromone. The schematic describing the underlying signaling mechanisms is included in FIG. 9.

The alternate inhibitory strains represent different OR operators with the particular operator determined by the recognized SBEs. If the producer and reporter strains both recognize the set of SBEs {P1, P2, . . . } and the inhibitory strain recognizes the set of SBEs {N1, N2, . . . }, then the system implements the operator (P1 OR P2 OR . . . ) AND NOT (N1 OR N2 OR . . . ). The following is the SBE assignment for this set of experiments. The producer strain sX406 and the reporter strains sX216 recognize the anti-FLAG M2 antibody. The inhibitory strain sX444 recognizes the anti-HA antibody. The inhibitory strain sX437 recognizes a dummy SBE denoted by SBE-X, where SBE-X represents any specific entity (non-specific interactions are assumed to be negligible) other than the anti-FLAG M2 antibody or the anti-HA antibody (or an entity with similar epitope specificity). Hence, the strain system sX406/sX216/sX444 implements the operator anti-FLAG M2 AND NOT anti-HA. Whereas the strain system sX406/sX216/sX437 implements the operator anti-FLAG M2 AND NOT SBE-X.

The above producer-reporter-inhibitor computing systems were tested on the BaHaF target objects to show the conformity of the inhibited cascade signaling network with the NEGATED IMPLICATION truth table. Three independent experiments were performed. The three strains sX406, sX216, and either sX444 or sX437 make up 43.1%, 43.1%, 13.8% of the total cell density, respectively. Identical Ba-HaF target objects were used in all cases with the exception of negative controls, where target objects are omitted altogether. The experiments were performed in triplicates and the results (see FIG. 31) are given in terms of measured fold-change as described in the PIC assay protocol. The output of the system evaluating the false proposition anti-FLAG M2 AND NOT anti-HA is comparable to the negative control (where the) and 2-fold lower than the output of the system evaluating the true proposition anti FLAG M2 AND NOT SBE-X.

### Example 11: Pheroimmuno Computing Assay in Liquid Medium with Immobilization

The following protocol is a modification of the PIC assay wherein object mobility is constrained. This protocol is an embodiment of the PIC architecture wherein the mixture objects are immobilized in a hydrogel matrix.

Preparatory aspects of the protocol are inherited from the PIC assay in liquid medium, which is described in the prequel. In some embodiments, computation is performed on antibody labeled beads, where the beads serve as target objects with known SBE profiles. Three different classes of beads are considered to demonstrate the basic logical operations: agarose beads covalently linked to anti-FLAG M2 antibodies (A2220 Sigma-Aldrich) denoted by (BaF), agarose beads covalently linked to anti-HA antibodies (26182 Thermo Fisher) denoted by (BaH), and polystyrene beads 15 μm in diameter covalently linked to anti-HA and anti-FLAG M2 antibodies (custom production by Spherotech inc.) denoted by (BaHaF).

Agarose beads were washed once in TBS and resuspended in TBS to a final concentration of 20,000 beads/ml. All beads were stored at 4° C. before use. All strains were grown overnight in YPD and diluted into SDC to 0.05 OD600 4 hrs before experiment. Prior to experiment cells were washed once in tris-buffered saline (TBS)+1% gelatin, washed a second time in TBS, and resuspended in TBS to 0.04 OD600.

The clustering reaction wherein the CUs and target objects are combined is modified to accommodate a 96-well plate format. In addition, non-specific background objects (8 μm polystyrene beads—SIGMA 78511-5ML-F) were included as an additional source of background noise. In an inert 96-well plate (BRAND—cat. n. 781900), combine 15 μl of collected sample (target object and background object mixture) with 5 μl of cell mixture and mix by pipetting up and down. Vortex the plate for 10 sec and incubate at 30° C. for 30 min with constant agitation (interrupted shaking at 550 rpm with 4 sec cycles) to cluster mixture objects.

The evaluation reaction continues in the same 96-well plate. Using a multichannel pipet, dilute each clustering reaction with 54 μl 2×SDC+1% Sodium alginate and 6 μl of 1 mM Nitrocefin to a final volume of 80 μl. Mix contents by light vortex for 3 mins. Immobilize mixture objects by adding 20 ul of 150 mM CaCl2 solution to each reaction. Vortex the plate for 10 sec and incubate reactions at 30° C. for 6 hrs (no agitation is required).

Following the 6 hr incubation period, analyze reaction readouts using 8-bit color images of each well. The readout is defined as the intensity measurement given by

readout=J_{cr}−J_{cg}−J_{cb} (8)

where J_{cr}, J_{cg}, and J_{cb} are the average intensities (using approx. the center most 20% of the image) measured in the red, green, and blue channels, respectively. Here, relative intensity values are used.

Control reactions are placed in the outermost wells and 2D interpolation is applied to adjust the absolute intensity measurements.

### Example 12: Evaluation of PIP with Signal Correction

In the following experiments, evaluation of PIPs with signal correction is demonstrated in 96-well PIC assay with immobilization. In the presented embodiments a single SBE is transposed into a single signal object and corrected with secondary signaling. The essential feature demonstrated in this study is the ability to correct PIPs with cluster specific regulation by supplementary CUs. This is accomplished by implementing feedback signaling networks. The study also demonstrates PIC in a 96-well plate format.

Two cellular CUs make up the system, the strains sX384 and sX387. In the case of a single PIP the CU functions are symmetric, i.e., both strains produce and recognize pheromone and beta-lactamase reporters. Each CU secretes their respective pheromone at some basal level. Upon recognition of their complementary pheromone, the CUs increase their pheromone production rates and further begin to secrete reporter peptides. The underlying signaling mechanism generates a positive feedback network whereby better discrimination between positive samples and negative controls is attained. The schematic describing the underlying signaling mechanisms is included in FIGS. 15A and 15B (the two CU format architecture).

The two-strain system was tested by PIC assay with immobilization on BaF target objects to study the dynamic and fold-change characteristics. In addition, the 96-well plate format was used to collect better statistical data regarding the system response. The experiment was performed in 18 replicates for both the positive samples and for the negative controls, where in the negative controls the BaF target objects are omitted. The time response of the evaluation reactions is shown in FIG. 32 where the definition of the readout is given in the PIC assay with immobilization protocol. Time-lapse RGB images converted to grayscale are further shown at 1 hour intervals in FIG. 33. Positive samples and negative controls become discernible after a 4 hr incubation period. The fold-increase in readout relative to negative controls reaches a maximum at 6 hrs with the maximum value ranging from 6- to 10-fold increase. In comparison, PIP evaluation without signal correction amounted to a 2-fold increase.

### Example 13: Modification of Molecular CUs for Efficient SBE Recognition

In this example, a molecular CU was modified to include an efficient SBE. The molecular CU used was anti-bovine serum albumin (BSA) immunoglobulin G (IgG), though other immunoglobulins (with different antigen specificity and/or class) can be used. Additional suitable molecular CUs include any molecule covalently or non-covalently linked to an amine group. The anti-BSA IgG was modified by chemical conjugation with a trans-cyclooctene (TCO) group via a selectable polyethylene glycol (PEG)-N-hydroxysuccinimide (NHS) linker, thereby adding a second binding moiety capable of recognizing tetrazine (TZ). Such an antibody is also referred to as a TCO-Ab. The addition of such a small chemical group inert towards macrobiological molecules results in a CU with reduced potential for inducing an undesired immune response to the CU as compared to addition of larger and/or more reactive groups. Other suitable CU modifications may include conjugation with various functional groups such as high efficiency conjugation entities, hydrophilic modulators, hydrophobic modulators, fluorescent probes, semiconductor particles, hydrogel adaptors, and the like.

To prepare these SBE-modified CUs, polyclonal anti-BSA antibody (A11133, Thermo Fisher Scientific) was conjugated with TCO-PEG(4)-NHS (TCO1010.0010, Iris Biotech). For the purpose of antibody purification, an Amicon Ultra-0.5 centrifugal filter unit with 50 kDa cutoff (UFC505096, Sigma) was washed once with 500 μl of PBS+0.005% Tween-20 and loaded with a solution of 450 μl PBS+0.005% Tween-20+50 μl of anti-BSA antibody. The loaded filter unit was washed four times with PBS+0.005% Tween-20, and the antibody was collected by final spin down with a collection tube, resulting in approx. 90 μl of antibody in PBS+0.005% Tween-20. The collected antibody solution was mixed with 13 μl of 50 mM TCO-PEG(4)-NHS solution and incubated for 2 hours at room temperature. The resulting modified antibody was washed four times with PBS+0.005% Tween-20 and concentrated to a final concentration of 1 mg/ml.

IgG was also modified by chemical conjugation with a dibenzocyclooctyne (DBCO) group, thereby adding a second binding moiety capable of recognizing azide-containing molecules. Such an antibody is also referred to as a DBCO-Ab. To prepare these SBE-modified CUs, polyclonal anti-BSA antibody (A11133, Thermo Fisher Scientific) was conjugated with DBCO-PEG4-NHS (A134, Click Chemistry). For the purpose of antibody purification, an Amicon Ultra-0.5 centrifugal filter unit with 50 kDa cutoff (UFC505096, Sigma) was washed once with 500 μl of PBS+0.005% Tween-20 and loaded with a solution of 450 μl PBS+0.005% Tween-20+50 μl of anti-BSA antibody. The loaded filter unit was washed four times with PBS+0.005% Tween-20, and the antibody was collected by final spin down with a collection tube, resulting in approx. 90 μl of antibody in PBS+0.005% Tween-20. The collected antibody solution was mixed with 13 μl of 50 mM DBCO-PEG4-NHS solution and incubated for 2 hours at room temperature. The resulting modified antibody was washed four times with PBS+0.005% Tween-20 and concentrated to a final concentration of 1 mg/ml.

### Example 14: Modification of SBE on the Surface of Cellular CUs

In this example, base SBEs of a cellular CU were modified. The base SBEs serve as intermediate binding sites for other functional elements. Such functional elements include, but are not limited to, high efficiency chemical conjugation elements, hydrophilic modulators, hydrophobic modulators, fluorescent probes, semiconductor particles, hydrogel adaptors, and the like. Genetically engineered yeast cells were used as the cellular CUs, and the base SBEs were glycophosphatidylinositol (GPI)-anchored proteins. The GPI-anchored proteins were modified by introduction of the hydrophilic modulator mPEG-SCM 2000 kDa (MF001024-2K, Biochempeg) and the high efficiency chemical conjugation moiety tetrazine-PEG5-NHS ester (1143-10, Click Chemistry).

Yeast cells (strain sX387) were grown overnight in SDC media and diluted into YPD media to an OD₆₀₀ of 2.0 immediately before modification. The cells were subsequently washed three times in PBS and a 100 μl cell suspension in PBS having an OD₆₀₀ of 2.0 was prepared. A conjugation mixture was prepared beforehand containing 5 μl of 30 mM mPEG-SCM 2000 kDa (MF001024-2K, Biochempeg) and 0.5 μl of 50 mM tetrazine-PEG5-NHS ester. The conjugation mixture and cell suspension were combined and incubated for 30 minutes at room temperature, and the cells were then washed three times in PBS+0.1% gelatin. The resulting modified cellular CUs were suitable for use directly in PIC reactions.

GPI-anchored proteins of yeast cells were also modified by introduction of the hydrophilic modulator mPEG-SCM 2000 kDa (MF001024-2K, Biochempeg) and either Azido-PEG4-NHS ester (AZ103-25, Click Chemistry) or methyltetrazine-PEG4-NHS ester (1069-10, Click Chemistry). Yeast cells (strain sX387) were grown overnight in SDC media and diluted into YPD media to an OD600 of 2.0 immediately before modification. The cells were subsequently washed three times in PBS and a 100 μl cell suspension in PBS having an OD600 of 2.0 was prepared. Conjugation mixtures were prepared beforehand containing 5 μl of 30 mM mPEG-SCM 2000 kDa (MF001024-2K, Biochempeg) and either 0.5 μl of 250 mM Azido-PEG4-NHS ester (AZ103-25, Click Chemistry) or 0.5 μl of 50 mM methyltetrazine-PEG4-NHS ester (1069-10, Click Chemistry). The respective conjugation mixture and cell suspension were combined and incubated for 30 minutes at room temperature, and the cells were then washed three times in PBS+0.1% gelatin. The resulting modified cellular CUs were suitable for use directly in PIC reactions.

### Example 15: Efficient Clustering of Cellular CUs with Other Cellular Objects

This example demonstrates a two-step method for the efficient preparation of specific and robust computational CU clusters. Critical parameters identified for sensitive and robust PIC reactions include sufficient representation of CUs in computational clusters and low formation of non-specific CU clusters, e.g., clusters of CUs that form spontaneously. In addition, to permit easy handling, for instance mixing, vortexing and liquid transfer, robustness of computational clusters was also found to be important.

Cellular CUs were clustered with other cellular objects in two steps. In step 1, cellular objects in a collected sample were non-covalently bound to molecular CUs, such as modified antigen-specific IgGs (see, e.g., Example 13), using standard immunochemical labelling to generate cellular object complexes. In step 2, cellular CUs were mechanically forced to interact with the cellular object complexes and subsequently resuspended as in Example 6.

The method was carried out for three different putative orthogonal binding pairs of cellular CUs and complexed cellular objects (Table 8). TCO-Ab and DBCO-Ab IgGs were prepared as in Example 13 and cellular CUs were prepared as in Example 14 using yeast strain sX387 modified with mPEG-SCM 2000 kDa (MF001024-2K, Biochempeg) and Azido-PEG4-NHS ester (AZ103-25, Click Chemistry), tetrazine-PEG5-NHS ester (1143-10, Click Chemistry), also referred to as hTet, or methyltetrazine-PEG4-NHS ester (1069-10, Click Chemistry), also referred to as mTet.

TABLE 8
Putative orthogonal pairs of cellular CUs/complexed cellular objects

| Binding Pair | Cellular CU | Complexed Cellular Object |
|---|---|---|
| hTet-TCO | hTet-CU | TCO-Ab |
| mTet-TCO | mTet-CU | TCO-Ab |
| Azide-DBCO | Azide-CU | DBCO-Ab |

Cluster formation with putative orthogonal and non-orthogonal pairings of cellular CUs and complexed cellular objects is shown in FIG. 34. Pairs hTet-TCO, mTet-TCO, and Azide-DBCO all generated clusters with high representation of cellular CUs. Pairs Azide-TCO and mTet-DBCO did not result in cellular CU/complexed cellular object clustering, demonstrating complete orthogonality for Azide-DBCO and mTet-TCO. However, pair hTet-DBCO exhibited non-specific clustering. It was also observed that hydrophilic modification of cellular CUs reduced spontaneous clustering between cellular CUs (data not shown).

The robustness of cluster formation was also evaluated. The two-step method described above (original method) was compared to an alternate method where step 1 and step 2 were interchanged (alternate method). In step 1 of the alternate method, modified cellular CUs were covalently linked to modified antigen-specific IgGs by standard methods. In step 2 of the alternate method, the newly formed complex and antigen presenting cellular objects were mechanically forced to interact and subsequently resuspended as in Example 6. As shown in FIG. 35, both methods can yield clusters with high cellular CU representation. However, whereas clusters formed by the original method remained intact even after rigorous agitation by vortexing, clusters formed by the alternate method were fragile and disassembled after even mild agitation (data not shown).

### Example 16: Signaling Between Cellular CUs and a Sample

This example demonstrates signaling between cellular CUs and a sample as determined by assays characterizing the recognition of external signals by cellular CUs and recombinant production by cellular CUs of signals recognizable as signals produced by cells in the sample. Signal-recognizing cellular CUs were modified to recombinantly express a heterologous SBE and/or to recombinantly express proteins with amino acid sequences equivalent to those capable of being produced by cells in the sample, and further modified to enable coupling of any heterologous SBEs to internal signal transmission. This configuration enabled omnidirectional signaling between the cellular CUs and the sample.

Signal-recognizing cellular CUs were prepared by modifying background strain rsX291 to i) constitutively express different heterologous human g-protein coupled receptors (SSTR2, NTSR1, or C5AR1); and ii) express in an inducible autocrine manner a ligand of their respective receptor (SST-14, NTS, or C5a), resulting in strains rsX420 (C5AR1/C5A), rsX626 (SSTR2/SST-14), and rsX769 (NTSR1/NTS), allowing for detection of external signals by activation of the GPCRs' native signaling pathways. Background strain rsX291 was also modified to express only a GPCR without the corresponding ligand, resulting in strains rsX324 (NTSR1), rsX389 (C5AR1), and rsX544 (SSTR2). As described in more detail below, a positive readout was detected for induced cellular CUs expressing a functional GPCR-ligand pair (induced rsX420, rsX626, and rsX769), but not for strains expressing only a GPCR (rsX324, rsX389, and rsX544, and uninduced rsX420, rsX626, and rsX769). The readout measured was beta-lactamase concentration as determined by kinetic assay.

For each strain an overnight culture was grown in YPD, diluted in the morning to an OD600 of ˜0.05, and incubated for another 4 hours. Next, for each strain, a master stock for the experiment was prepared as follows. The cells were washed with water and resuspended into SDC media to an OD600 of 0.05. The prepared strains were then transferred into a 96-well microplate and 10 μl of 1 mM nitrocefin (484400 Sigma-Aldrich) was added to each sample. To the samples for the induced condition, 2 μl of a 1 g/L doxycycline stock (D1822 Sigma-Aldrich) was added, and 2 μl of ddH₂0 was added to the samples for the uninduced condition. The beta-lactamase concentration for each sample was quantified using a microplate reader at 30° C. by measuring absorbance at wavelengths 486 nm (A₄₈₆) and 700 nm (A₇₀₀) every 2 minutes for 2 hours. Specifically, the beta-lactamase concentration was quantified between 60 min and 80 min.

As shown in FIG. 36, induced strains expressing a functional GPCR-ligand pair (induced rsX420, rsX626, and rsX769) showed a positive readout ranging from a 20- to 30-fold change from their respective uninduced condition. Uninduced strains expressing only a GPCR (uninduced rsX420, rsX626, and rsX769), singly modified strains expressing only a GPCR (rsX324, rsX389, and rsX544), and the background strain (rsX291) showed only a negligible readout. These results demonstrate that the production of functional levels of external signals by cellular CUs can be achieved. Finally, it was shown that the detection window is reasonably long, with measurable activation between 60 min and 80 min for all GPCR-ligand pairs tested. By introducing other heterologous SBEs, the range of detectable external signals can be expanded.

### Example 17: Clinically Relevant Analysis of Surface Profiles

The following example demonstrates application of PIC towards clinically relevant characterization of sample object surface profiles (e.g., cell surface profiles). In some cases, sample objects are from a tissue sample comprising a heterogeneous population of objects suspended in a liquid medium, e.g., cells and cell aggregates originating from a solid or liquid tissue prepared in a synthetic buffer. The presented embodiments illustrate the utility of PIC in obtaining data that lead to information including, but not limited to, sample object count, origin, potential localization, replication rate, drug sensitivity, drug action, and pluripotency.

The sample to be characterized is combined with a prepared mixture of cellular and molecular computing units in a PIC assay as described herein. In some cases, computing units of different types are included. In one example, 1, 2, 3, 4, 5, or more different types of computing units are combined so that multiple copies of each computing type are present. In some cases, computing units include SBEs that have cognate binding partners that are SBEs located on surfaces of select sample objects. In one example, CU type 1 (CU1), CU type 2 (CU2), CU type 3 (CU3), CU type 4 (CU4), and CU type 5 (CU5) include SBEs that bind to possibly overlapping subsets of SBEs located on some sample objects, e.g., markers of type 1 (M1), markers of type 2 (M2), markers of type 3 (M3), markers of type 4 (M4), markers of type 5 (M5), respectively. In some cases, computing units produce subsets of signal objects, e.g., SO1, SO2, SO3, and SO4 corresponding to CU1, CU2, CU3, and CU4, respectively. In some cases, computing units include one or more SBEs that recognize members of the signal object subsets, e.g., R1, R2, R3, and R4 corresponding to subsets SO1, SO2, S03, and SO4, respectively. In some cases, a computing unit's production of an SO is enhanced if the computing unit includes an SBE and that SBE binds to its cognate binding partner, e.g., if the computing unit includes R1, R2, R3, or R4 and if the SBE binds to SO1, SO2, SO3, or SO4, respectively. In some cases, computing units degrade signal objects. In one example, CU5 degrades all signal objects in subsets SO1, SO2, SO3, and SO4.

The mixture of computing units is configured to perform a specific function. In one example, to gather data regarding sample objects bound to M1 and M2, CU1 and CU2 are used. In some cases, subsets M1 and M2 are different, and in other cases, subsets M1 and M2 are the same, and such objects are referred to as target objects. In some cases, signal object production is configured as shown in FIG. 10, such that CU2 includes R1. In some cases, signal correction as shown in FIG. 15B is used. In some such cases, SO2 includes a readout signal.

In this case, three types of clusters are self-assembled upon proper mixing: CT1 where CU1s are clustered with CU2s and target sample objects, CT2 where CU1s are clustered with sample objects bound only to M1, and CT3 where CU2s are clustered with sample objects bound only to M2. Incomplete clusters, where correspondence between sample object SBEs and computing units is not conserved, are unlikely if a) SBE-associated sample objects are of equal size or larger in comparison to the computing units and b) the number of computing unit copies is sufficiently greater than the number of SBE-associated sample objects, and such incomplete clusters are not be considered in the model. In CT1 clusters, CU1 is capable of enhancing SO2 production by CU2, resulting in production of a readout SO. The number of clusters formed, and thereby the concentration of clusters in the reaction volume, depends on the number of target sample objects present.

In another example, to gather data regarding sample objects bound to M1, M2, and M3 (such sample objects referred to as target sample objects), CU1, CU2, and CU3 are used. In some cases, signal object production is configured such that CU2 includes R1 and CU3 includes R2. In some cases, CU1 includes R3 for signal correction. In some such cases, SO3 includes a readout signal.

In this case, seven types of clusters are self-assembled upon proper mixing. Only complete clusters are likely by the same considerations as described above. Out of the seven types of clusters, only target sample objects yield a cluster where SO3 production is enhanced and the readout SO is sufficiently produced. Again, the number of clusters formed, and thereby the concentration of clusters in the reaction volume, depends on the number of target sample objects present.

In some cases, collection of data is multiplexed. In one example, to gather data regarding sample objects bound to M1, M2, and M3 (target objects 1) and M1, M2, and M4 (target objects 2), CU1, CU2, CU3, and CU4 are used. In some cases, signal object production is configured so that CU2 includes R1 and both CU3 and CU4 include R2. In some cases, CU1 includes R3 and R4 for signal correction. In some such cases, SO3 includes a readout signal 1 and SO4 includes a readout signal 2. In some cases, the readout signals 1 and 2 are selected so that independent measurement of each signal is possible.

In this case, fifteen types of clusters are self-assembled upon proper mixing. Only complete clusters are likely by the same considerations as described above. Out of the fifteen types of clusters, only target objects 1 and target objects 2 yield clusters where SO3 and/or SO4 production is enhanced and thereby a readout signal 1 and/or readout signal 2 is sufficiently produced. Again, the number of clusters formed depends on the number of target objects of each type present. With this configuration, sample objects associated with SBEs M1, M2, M3, and M4 are counted as both target objects 1 and 2.

In some cases, an additional computing unit CU5 that degrades all signal objects is used to gather data regarding sample objects that are not associated with a particular SBE, e.g., sample objects bound to any combination of SBEs M1, M2, M3, or M4 but not to SBE M5. In some of the cases described above this additional negation is included, e.g., without making changes to any of the other computing units.

In some cases where the absence of an SBE is necessary, clusters comprising sample objects associated with the negating SBE are assembled first, e.g., by properly mixing CU5s with all sample objects before adding other CUs. In other such cases, all clusters are self-assembled at the same time by properly mixing all CUs and sample objects. With the inclusion of SO-degrading CUs, an additional set of clusters is formed, e.g. clusters that include CU5s. In these clusters, signal production is not enhanced by SOL SO2, SO3, or SO4. As such, readout signals are not produced in any of these compositions.

In some cases, the sample SBE subsets, e.g., M1, M2, M3, M4, and M5, belong to marker families indicating the object origin, e.g., c-Met, BMI-1, CD340, and the like; the object growth potential, e.g., CSV, CD-133, E-Cadherin, and the like; the object pluripotency, e.g., CD34, CD44, CD133, CD271, and the like; the object action, e.g., CD274, CD133, CD90, CD44, and the like; and/or possible object localization, e.g., CD184, VCAM1, L1CAM, and the like.

In one example, the origin of cancer cells is identified more precisely using a combination of surface markers, such as for hematological cancer cells (with exemplary markers including one or more of CD34, CD38, CD19, and CD26), breast cancer cells (with exemplary markers including one or more of CD44, CD24, CD29, and CD133), colon cancer cells (with exemplary markers including one or more of CD44, CD24, CD26, CD29, CD133, CD166, and Ep-CAM), brain cancer cells (with exemplary markers including one or more of CD90, CD133, and CD15), head and neck cancer cells (with exemplary markers including one or more of CD44 and CD271), liver cancer cells (with exemplary markers including one or more of CD44, CD90, CD133, CD13, and Ep-CAP), lung cancer cells (with exemplary markers including one or more of CD44, CD133, and CD166), pancreas cancer cells (with exemplary markers including one or more of CD44, CD24, and CD133), prostate cancer cells (with exemplary markers including one or more of CD44, CD24, CD133, CD166, and CD151), esophagus cancer cells (with exemplary markers including one or more of CD271, CD44, CD24, and CD90), cervix cancer cells (with exemplary markers including one or more of CD13, CD29, CD44, and CD105), or stomach cancer cells (with exemplary markers including one or more of CD44 and CD133).

In some cases, for such subsets a mixture of cellular and molecular computing units is used. In one example, CU1, CU2, CU3, CU4, and CU5 are cellular computing units. In this example, the computing units are covalently or noncovalently bound to SBEs with cognate binding partners M1, M2, M3, M4, and M5, respectively. In some cases, these include bispecific antibodies in complex with a GPI-anchored protein, e.g. anti-M1/anti-HEMA in complex with HEMA-Flo11, anti-M2/anti-FLAG in complex with FLAG-Flo11, anti-M3/anti-Myc in complex with cMyc-Flo11, anti-M4/anti-V5 in complex with V5-Flo11, and anti-M5/anti-Etag in complex with Etag-Flo11, where anti-M1, anti-M2, anti-M3, anti-M4, and anti-MS denote antibodies that recognize SBEs from the subsets M1, M2, M3, M4, and M5, respectively. In some cases, signal objects are diffusible molecules, e.g., from the family of yeast mating factors and in particular from organisms such as Saccharomyces Cerevisiae, Candida albicans, Candida glabrata, Paracoccidioides brasiliensis, Histoplasma capsulatum, Lodderomyces elongisporus, Botrytis cinerea, Fusarium graminearum, Magnaporthe oryzae, Zygosaccharomyces bailii, and Zygosaccharomyces rouxii. In some cases, SBEs associated with SOs and enhancing SO production include membrane-bound receptors, e.g., from the family of GPCRs and in particular mating factor receptors from yeast species such as Saccharomyces Cerevisiae, Candida albicans, Candida glabrata, Paracoccidioides brasiliensis, Histoplasma capsulatum, Lodderomyces elongisporus, Botrytis cinerea, Fusarium graminearum, Magnaporthe oryzae, Zygosaccharomyces bailii, and Zygosaccharomyces rouxii. In some cases, readout SOs include various molecular reporters, e.g., fluorescent proteins, proteases, luciferases, lactamases, and the like. In some cases, multiplexing is achieved with independently measurable molecular reporters, e.g., GFP/CFP/YFP, protease variants, luciferase orthologues, ELISA peptide screens, and the like.

In some of the cases described above, cellular computing units are replaced by molecular computing units that produce signal objects by activating inactive signal objects as shown in FIG. 16. In one example, CU1, CU2, CU3, or CU4 is replaced by an SBE-bound enzyme E1, E2, E3, or E4, respectively. In some cases, an enzyme from a family of proteases (e.g., from the family of threonine, aspartic, cysteine, metallo, or serine proteases including, but not limited to, TEV protease, enteropeptidase, HCV protease, trypsin, chymotrypsin, ULP1, and NEDP1) is used. In this case, signal production is enhanced by the presence of inactive polypeptide signals and signal objects are produced by proteolysis of these signals (e.g., cleavage of an extended N-terminus or C-terminus). In cases where the molecular computing unit replaces a cellular computing unit that produces SOs constitutively, e.g., CU1, the inactive signal objects are external signal objects that are added. In cases where the molecular computing unit replaces a cellular computing unit whose SO production is enhanced by the presence of another SO, the inactive signal objects are either secreted by the preceding cellular CUs or produced from inactive signal objects by preceding molecular CUs. In one example, to characterize sample objects comprising SBEs M1 and M2, two protease computing units CU1 and CU2 are used, where CU1 cleaves an inactive external signal object to produce SO1. In some cases, SO1 is then further cleaved by CU2 to produce a quantifiable signal object SO2. In some such cases, the cleavage site recognized by CU2 is occluded in its inactive precursor. Specific quantification of SO2 is carried out, for example, by an immunochemical (e.g., ELISA) or size-based assay.

In some of the cases described above, CU1, CU2, CU3, and/or CU4 are replaced by an SBE-bound zymogen Z1, Z2, Z3, and/or Z4, respectively. In some cases, an enzyme from a family of protease zymogens (e.g., a member of the family of serine proteases) is used, where a zymogen is activated either by proteolysis or complementation. In this case, the signal objects are zymogens as well (e.g., a member of protease zymogens). In some cases, signal objects are freely diffusible external zymogens added to the medium. In some cases where the molecular computing unit replaces a cellular computing unit that produces SOs constitutively, e.g., CU1, the zymogen is from a family of split proteases that is reconstituted upon recognition (e.g., a split TEV protease linked to an SBE) of the corresponding SBE, e.g., M1. In some cases where the molecular computing unit replaces a cellular computing unit whose SO production is enhanced by the presence of another SO, the CU is taken from a family of protease precursors that are activated by removal of their prodomains (e.g., a member of the serine protease family). In this case, the enhancing SO is an active protease that is either secreted by the preceding cellular CU or activated by the preceding zymogen CU. In one example, to characterize sample objects comprising SBEs M1 and M2, CU1 is an SBE-bound protease and CU2 is an SBE-bound zymogen. In this case, CU1 activates a diffusible zymogen to produce SO1. Subsequently SO1 activates the CU2 by removal of its prodomain. Lastly, CU2 produces SO2 by activating a second diffusible zymogen. Characterizing sample objects associated with M1, M2, and M3 or M1, M2, and M4 proceeds in the same way. In some cases, readout signals that are active proteases are quantified, for example, by colorimetric or fluorescent measurements using available protease-specific substrates.

In some of the above examples, a PIC assay is prepared as described in Example 6 or Example 11. In some cases, prior to the PIC assay, the two-step method described in Example 15 is used, where additional computing unit types are introduced in the first step to transpose sample object SBEs. In one example, to transpose M1, M2, M3, and M4, additional computing units CU1b, CU2b, CU3b, and CU4b, respectively, are introduced. Exemplary SBE pairs include a) anti-M1-biotin antibody (CU1b) and streptavidin-CU1, b) anti-M2-TCO antibody (CU2b) and methyltetrazine-CU2, c) anti-M3-DBCO antibody (CU3b) and azide-CU3, and e) anti-M4-BG (CU4b) and SNAP-CU4.

In some cases, the measurement of readout signals is carried out to yield data regarding specific sample object features, e.g., count, origin, potential localization, replication rate, drug sensitivity, drug action, and pluripotency. In one example, to detect the presence of target objects, a single reaction is prepared and readout signal measurement is carried out, where a readout signal measurement above a fixed threshold is indicative of the presence of the target objects. In another example, to count the number of sample objects comprising a specific combination of SBEs, the PIC reaction is aliquoted into discrete volumes. The number of aliquots depends on the expected maximum number of target objects. The number of aliquots generating a positive readout, i.e., a readout exceeding a fixed threshold, gives a measurement of target object count.

In another example, to measure the average replication rate of sample objects comprising a specific combination of SBEs, a PIC assay is performed at different points in time. At the first time point, the sample objects are split into two equal volumes and the number of target objects in the first volume is counted. The second half of the sample is incubated using standard practices for the duration of at least a single division cycle. Following the incubation period, at a second time point, the number of sample objects in the second volume is counted. The difference in the numbers counted at the two time points relative to the time interval provides a measurement of the growth rate. In some cases, normalization data is gathered by simultaneously measuring the number of conserved sample objects.

In another example, to measure the sensitivity of sample objects comprising a specific combination of SBEs to an external agent, e.g., various types of therapy or modulation including, but not limited to, small molecule drugs, hormones (e.g. testosterone), neurotransmitters (e.g. dopamine), peptidic hormones (e.g., somatostatin), neuropeptides (e.g., neurotensin), glycoproteins (e.g., TSH), chemokines (e.g. CCL5), interleukins (e.g. IL1b), and cell therapies, a PIC assay is performed at different points in time under different conditions. At the first time point, the sample objects are split into three equal volumes and the number of target objects in the first volume is counted. The second volume of the sample is incubated with the particular agent for a fixed period of time. The third volume of the sample is incubated with a control agent for the same period of time. Following the incubation period, at the second time point, the number of sample objects in the second and third volumes are counted. The difference in the numbers counted in the second and third volumes relative to the number counted in the first volume is used to measure the efficacy of the particular agent in performing the intended function.

### Example 18: Analysis of Secretomes

The following example demonstrates application of PIC towards clinically relevant characterization of molecules secreted by specific sample objects into the medium (secretomes). In some cases, sample objects are from a tissue sample comprising a heterogeneous population of objects suspended in a liquid medium, e.g., cells and cell aggregates originating from a solid or liquid tissue prepared in a synthetic buffer. The presented embodiment illustrate the utility of PIC in obtaining data that leads to information including, but not limited to, sample object state, modulation ability, and immunoglobulin specificity.

Secretome analysis proceeds as in Example 17. In addition to the elements described therein, in this example, at least one CU includes an SBE, e.g., RE1, that interacts with signal objects secreted by some sample objects, e.g., SOE1. In one example, to measure production of the signal object SOE1, CU1 includes RE1 such that production of SO1 is enhanced only in the presence of SOE1. If a single subset of SBEs, e.g., M1, is sufficient to characterize the surface profile of the target objects, a single CU, e.g., CU1, is sufficient. In this case the CU-produced signal, e.g., SO1, is used as the readout. If more than one SBE is required, enhanced production of SO1 enhances production of SO2 by CU2 as described in Example 17. In the absence of SOE1, production of SO1 is insufficient to enhance production of SO2 by CU2. In some such cases, CU1 includes a cellular computing unit, e.g., where CU1 comprises a yeast cell engineered to express RE1, and where RE1 belongs, e.g., to a GPCR family including, but not limited to, CCR2, CCR5, CXCR4, SSTR1, SSTR2, SSTR3, SSTR5, NTSR1,C5AR1, GLP1R, GHRHR, AGTR1, AGTR2, and C5AR1, and SOE1 belongs, e.g., to a peptide hormone family including, but not limited to, somatostatin, neurotensin, and angiotensin, or belongs, e.g., to a protein hormone family including, but not limited to, GHRH, GLP-1, and C5 anaphylatoxin.

In some cases, the measurement of readout signals in PIC assays that include secretome data yields information regarding object state, modulation ability, and/or immunoglobulin specificity. In one example, for colorectal tumor metastasis, expression of CCL5 (an SO here) is used to recruit platelets to assist CTCs in breaking through the subepithelial extracellular matrix at the point of invasion. Of interest are target objects with the following SBE profile: CD24+, CD166+, and CD133−(known from colorectal cancer cell lines), and also secreting CCL5. In some cases, detection/cell count of cells positive for CCL5 expression is used to estimate metastatic potential. In this case, using the above notation, SOE1 is CCL5, RE1 is CCR5, M1 is CD24, M2 is CD166, and M5 is CD133.

In another example, breast cancer metastatic cells use CCL2 (an SO here) to stimulate tumor associated macrophages to increase their metastatic potential. Of interest are target objects with the following SBE profile: CDH1+, CD31−, CD45−, and CD11b−, and also secreting CCL2. In some cases, detection/cell count of cells positive for CCL2 expression is used to estimate metastatic potential. In such a case, information about cell count of CCL2-expressing cells might be critical for properly timed therapy involving CCR2 blocking. Improper timing of such therapies has been shown to be detrimental to therapeutic efficacy. In this case, using the above notation, CCL2 is SOE1, CCR2 is RE1, M1 is CDH1, and M5 is the SBE set made up of CD11b, CD31, and CD45.

In one example, to detect the presence of target objects characterized by a surface profile and a secretome, a single reaction is prepared and readout signal measurement is carried out, where a readout signal measurement above a fixed threshold is indicative of sufficient signal production by the target sample objects. In some cases, to normalize the data and properly set the detection threshold, the sample is separated into two volumes. Subsequently, a first PIC assay is performed using the first volume to count the number of sample objects characterized by the specific surface profile. A second PIC assay is performed on the second volume to quantify the overall signal production by the target sample objects. In some cases, the PIC assay is discretized as described in Example 17 to count the number of signal producing sample objects. The PIC reaction is aliquoted into discrete volumes, where the number of aliquots depends on the expected maximum number of SO-producing target objects. The number of aliquots generating a positive readout, i.e., a readout exceeding a fixed threshold, gives a measurement of target object count.

Sequence Listing

| SEQ | | |
|---|---|---|
| ID | | |
| NO | Sequence | Description |
| 1 | DYKDHDGDYKDHDIDYKDDDDK | 3xFLAG tag |
| 2 | AEAAAKEAAAKEAAAKA | Linker |
| 3 | YPYDVPDYA | HA tag |
| 4 | TGATTCGGTA ATGCGCTGTA A | Barcode 1 |
| 5 | TAACTAACT | Barcode 2 |
| 6 | TAATACGCGA AACTCCAGTC G | Barcode 3 |
| 7 | TAATCAGGGA AAGATCCTTC G | Barcode 4 |
| 8 | TAAACTGAGC TGACCTAAGG G | Barcode 5 |
| 9 | TGATGACCCA CTCAGACATG A | Barcode 6 |
| 10 | TAGTTGGCCCACGAACCGTAA | Barcode 7 |
| 11 | TGATTCGGTAATGCGCTGTAA | Barcode 8 |
| 12 | TCCGGATGACACTCGACGCCATAGAT | Barcode 9 |
| 13 | TGAAAGATCAAGTTGTAGGCC | Barcode 10 |
| 14 | GAATGTGGTCTGTAC | Sequence |
| | | inserted |
| | | into GPA1 |
| 15 | CACCCATGAACCACACGGTTAGTCCAAAAGGGG | pTet |
| | CAGTTCAGATTCCAGATGCGGGAATTAGCTTGC | promoter |
| | TGCCACCCTCACCTCACTAACGCTGCGGTGTGC | |
| | GGATACTTCATGCTATTTATAGACGCGCGTGTC | |
| | GGAATCAGCACGCGCAAGAACCAAATGGGAAAA | |
| | TCGGAATGGGTCCAGAACTGCTTTGAGTGCTGG | |
| | CTATTGGCGTCTGATTTCCGTTTTGGGAATCCT | |
| | TTGCCGCGCGCCCCTCTCAAAACTCCGCACAAG | |
| | TCCCAGAAAGCGGGAAAGAAATAAAACGCCACC | |
| | AAAAAAAAAAAAATAAAAGCCAATCCTCGAAGC | |
| | GTGGGTGGTAGGCCCTGGATTATCCCGTACAAG | |
| | TATTTCTCAGGAGTAAAAAAACCGTTTGTTTTG | |
| | GAATTTCCCATTTCGCGGCCACCTACGCCGCTA | |
| | TCTTTGCAACAACTATCTGCGATAACTCAGCAA | |
| | ATTTTGCATATTCGTGTTGCAGTATTGCGATAA | |
| | TGGGAGTCTTACTTCCAACATAACGGCAGAAAG | |
| | AAATGTGAGAAAATTTTGCATCCTTTGCCTCCG | |
| | TTCAAGTATATAAAGTCGGCATGCTCCCTATCA | |
| | GTGATAGAGATCTCCCTATCAGTGATAGAGAGT | |
| | TCTAATTATTCTTATTCTCCTTTATTCTTTCCT | |
| | AACATACCAAGAAATTAATCTTCTGTCATTCGC | |
| | TTAAACACTATATCAATAAT | |

1.-87. (canceled)

88. A system for biological computing, comprising:
one or more computing units (CUs),
one or more sample objects derived from an input sample;
wherein the one or more CUs are configured to interact with the one or more sample objects and generate an output signal indicative of a characteristic of the input sample.

89. The system of claim 88, wherein the system is configured to form one or more computational clusters, wherein each computational cluster of the one or more computational clusters comprises, independently, one or more of the CUs,
wherein at least one of the one or more computational clusters further comprises, independently, one or more of the sample objects.

90. The system of claim 88, wherein each of the one or more CUs is, independently,
i) associated with one or more surface-bound entities (SBEs);
ii) capable of recognizing a signal object (SO);
iii) capable of producing an SO;
iv) capable of degrading an SO;
v) capable of producing a change in a material property of a medium comprising the system; and/or
vi) capable of producing another CU.

91. The system of claim 90, wherein an SBE associated with a CU in the system comprises an SO.

92. The system of claim 90, wherein association of a CU in the system with an SBE comprises covalent attachment of the SBE to a surface of the CU or non-covalent attachment of the SBE to a surface of the CU.

93. The system of claim 90, wherein a CU in the system is capable of producing an SO such that the SO is released into a medium comprising the system.

94. The system of claim 90, wherein the capability of a first CU in the system to produce a first SO is enhanced by binding of a first SBE associated with the first CU to its cognate binding partner, and/or
wherein the capability of a second CU in the system to produce a second SO is attenuated by binding of a second SBE associated with the second CU to its cognate binding partner.

95. The system of claim 90, wherein the capability of a first CU in the system to degrade a first SO is enhanced by binding of a first SBE associated with the first CU to its cognate binding partner, and/or
wherein the capability of a second CU in the system to degrade a second SO is attenuated by binding of a second SBE associated with the second CU to its cognate binding partner.

96. The system of claim 88, wherein each of the one or more CUs comprises, independently, a cell or a molecule,
wherein the molecule is selected from a polypeptide, a polypeptide derivative, a nucleic acid, and a solid support.

97. The system of claim 96, wherein the polypeptide is an enzyme, wherein the enzyme is capable of converting an agent in a medium comprising the system into an SO.

98. The system of claim 88, wherein each of the one or more sample objects is, independently,
i) associated with one or more SBEs;
ii) capable of recognizing an SO;
iii) capable of producing an SO;
iv) capable of degrading an SO;
v) capable of producing a change in a material property of a medium comprising the system; and/or
vi) capable of producing another sample object.

99. The system of claim 98, wherein recognition of the SO by the sample object is capable of inducing the sample object to produce another SO.

100. The system of claim 99, wherein each of the one or more sample objects is, independently, one of a cell, a molecule, and an SBE associated with the cell comprises an antigen on the surface of the cell.

101. The system of claim 88, comprising a logical OR module comprising a first CU, wherein the first CU comprises a first CU SBE and a second CU SBE, the first CU SBE is capable of binding to a first cognate binding partner and the second CU SBE is capable of binding to a second cognate binding partner, and
1) the first CU is capable of producing a first SO, binding of the first CU SBE to the first cognate binding partner enhances the capability of the first CU to produce the first SO and binding of the second CU SBE to the second cognate binding partner enhances the capability of the first CU to produce the first SO; or
2) the first CU is capable of degrading a first SO, binding of the first CU SBE to the first cognate binding partner enhances the capability of the first CU to degrade the first SO and binding of the second CU SBE to the second cognate binding partner enhances the capability of the first CU to degrade the first SO.

102. The system of claim 101, wherein the first cognate binding partner is a first sample object SBE associated with at least one of the one or more sample objects and the second cognate binding partner is a second sample object SBE associated with at least one of the one or more sample objects.

103. The system of claim 88, comprising a logical AND module comprising a first CU and a second CU, wherein
the first CU comprises a first CU SBE and the second CU comprises a second CU SBE, the first CU SBE is capable of binding to a first cognate binding partner and the second CU SBE is capable of binding to a second cognate binding partner, wherein
the first CU is capable of producing a first SO, the second CU is capable of producing or degrading a second SO, binding of the first CU SBE to the first cognate binding partner enhances the capability of the first CU to produce the first SO, and recognition of the first SO by the second CU enhances the capability of the second CU to produce or degrade the second SO, and wherein
clustering of the first and second cognate binding partners in a computational cluster enhances the effect of the first SO such that the first SO produced by the first CU bound to the first cognate binding partner is capable of inducing the second CU bound to the second cognate binding partner to produce or degrade the second SO.

104. The system of claim 103, wherein the first cognate binding partner is a first sample object SBE associated with a sample object in the system and the second cognate binding partner is a second sample object SBE associated with the sample object.

105. The system of claim 88, comprising a logical negated implication module comprising a first CU, a second CU, and a third CU, wherein
the first CU comprises a first CU SBE, the second CU comprises a second CU SBE, the third CU comprises the first CU SBE, the first CU SBE is capable of binding to a first cognate binding partner, and the second CU SBE is capable of binding to a second cognate binding partner, wherein
the first CU is capable of producing a first SO, the second CU is capable of degrading the first SO, the third CU is capable of producing or degrading a second SO, binding of the first CU SBE of the first CU to the first cognate binding partner enhances the capability of the first CU to produce the first SO, binding of the second CU SBE of the second CU to the second cognate binding partner enhances the capability of the second CU to degrade the first SO, and recognition of the first SO by the third CU enhances the capability of the third CU to produce or degrade the second SO, and wherein
clustering of the first cognate binding partners enhances the effect of the first SO such that the first SO produced by the first CU bound to the first cognate binding partner is capable of inducing the third CU bound to the first cognate binding partner to produce or degrade the second SO; and
clustering of the first and second cognate binding partners in a computational cluster enhances the effect of the second CU such that the second CU bound to the second cognate binding partner is capable of attenuating induction of third CU by degrading the first SO produced by the first CU bound to the first binding partner.

106. The system of claim 105, wherein the first cognate binding partner is a first sample object SBE associated with a sample object in the system and the second cognate binding partner is a second sample object SBE associated with the sample object.

107. The system of claim 88, wherein a CU in the system is configured to respond to an SO in the system by enhancing the level of the SO, wherein the CU is capable of producing the SO, and interaction of the SO with the CU enhances the capability of the CU to produce the SO.

108. The system of claim 107, wherein the CU is configured to respond to the SO by producing another SO, and wherein interaction of the other SO with another CU that is capable of producing the SO enhances the capability of the other CU to produce the SO.

109. The system of claim 88, wherein a CU that is not localized to a computational cluster in a medium comprising the system is configured to degrade an SO produced by another CU present in the computational cluster.

110. The system of claim 88, wherein the system further comprises an agent that increases the viscosity of a medium comprising the system.

111. The system of claim 88, wherein the characteristic of the input sample comprises an indication of whether a target object is present or absent in the input sample and/or a quantification of the level of the target object in the input sample.

112. The system of claim 111, wherein the input sample is a biological sample derived from an individual.

113. The system of claim 112, wherein the target object is a target cell or a pathogen.

114. The system of claim 113, wherein the target cell is one of a disease cell, a fetal cell, a stem cell and a state-specific target cell.

115. The system of claim 112, wherein the target object is indicative of a disease or condition in the individual, and the characteristic of the biological sample further comprises a diagnosis of the disease or condition in the individual.

116. A method of detecting the presence or absence of a target object in an input sample comprising:
a) incubating the composition of claim 111 for a sufficient amount of time for the output signal to be generated; and
b) detecting the output signal, thereby detecting the presence or absence of the target object in the input sample.

117. A method of diagnosing a disease or condition in an individual, comprising:
a) incubating a composition comprising the system of claim 111 for a sufficient amount of time for the output signal to be generated; and
b) detecting the output signal, thereby diagnosing the disease or condition in the individual.


SEQUENCE LISTING (15 sequences)

SEQ ID NO: 1 - protein, 22 aa, Artificial sequence
  Synthetic polypeptide
DYKDHDGDYKDHDIDYKDDDDK

SEQ ID NO: 2 - protein, 17 aa, Artificial sequence
  Synthetic polypeptide
AEAAAKEAAAKEAAAKA

SEQ ID NO: 3 - protein, 9 aa, Artificial sequence
  Synthetic polypeptide
YPYDVPDYA

SEQ ID NO: 4 - DNA, 21 bp, Artificial sequence
  Synthetic polynucleotide
tgattcggtaatgcgctgtaa

SEQ ID NO: 5 - DNA, 9 bp, Artificial sequence
  Synthetic polynucleotide
taactaact

SEQ ID NO: 6 - DNA, 21 bp, Artificial sequence
  Synthetic polynucleotide
taatacgcgaaactccagtcg

SEQ ID NO: 7 - DNA, 21 bp, Artificial sequence
  Synthetic polynucleotide
taatcagggaaagatccttcg

SEQ ID NO: 8 - DNA, 21 bp, Artificial sequence
  Synthetic polynucleotide
taaactgagctgacctaaggg

SEQ ID NO: 9 - DNA, 21 bp, Artificial sequence
  Synthetic polynucleotide
tgatgacccactcagacatga

SEQ ID NO: 10 - DNA, 21 bp, Artificial sequence
  Synthetic polynucleotide
tagttggcccacgaaccgtaa

SEQ ID NO: 11 - DNA, 21 bp, Artificial sequence
  Synthetic polynucleotide
tgattcggtaatgcgctgtaa

SEQ ID NO: 12 - DNA, 26 bp, Artificial sequence
  Synthetic polynucleotide
tccggatgacactcgacgccatagat

SEQ ID NO: 13 - DNA, 21 bp, Artificial sequence
  Synthetic polynucleotide
tgaaagatcaagttgtaggcc

SEQ ID NO: 14 - DNA, 15 bp, Artificial sequence
  Synthetic polynucleotide
gaatgtggtctgtac

SEQ ID NO: 15 - DNA, 713 bp, Artificial sequence
  Synthetic polynucleotide
cacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaatttcccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgctccctatcagtgatagagatctccctatcagtgatagagagttctaattattcttattctcctttattctttcctaacataccaagaaattaatcttctgtcattcgcttaaacactatatcaataat